SEQUENCE LISTING (18 sequences)

SEQ ID NO: 1 - DNA, 23 bp, Artificial Sequence
  Human p63 FW primer
gacaggaaggcggatgaagatag

SEQ ID NO: 2 - DNA, 24 bp, Artificial Sequence
  Human p63 RV primer
tgtttctgaagtaagtgctggtgc

SEQ ID NO: 3 - DNA, 21 bp, Artificial Sequence
  Human Ki67 FW primer
gaggtgtgcagaaaatccaaa

SEQ ID NO: 4 - DNA, 23 bp, Artificial Sequence
  Human Ki67 RV primer
ctgtccctatgacttctggttgt

SEQ ID NO: 5 - DNA, 21 bp, Artificial Sequence
  Human KRT13 FW primer
gaccgccaccattgaaaacaa

SEQ ID NO: 6 - DNA, 22 bp, Artificial Sequence
  Human KRT13 RV primer
tccaggtcagtcttagacagag

SEQ ID NO: 7 - DNA, 23 bp, Artificial Sequence
  Human KRT4 FW primer
ctctttgagacctacctcagtgt

SEQ ID NO: 8 - DNA, 19 bp, Artificial Sequence
  Human KRT4 RV primer
ggctgctgtgcgtttgttg

SEQ ID NO: 9 - DNA, 21 bp, Artificial Sequence
  Human EGFR FW primer
aggcaggagtaacaagctcac

SEQ ID NO: 10 - DNA, 21 bp, Artificial Sequence
  Human EGFR RV primer
atgaggacataaccagccacc

SEQ ID NO: 11 - DNA, 20 bp, Artificial Sequence
  Human Actin FW primer
tgcgtgacattaaggagaag

SEQ ID NO: 12 - DNA, 20 bp, Artificial Sequence
  Human Actin RV primer
tgaaggtagtttcgtggatg

SEQ ID NO: 13 - DNA, 21 bp, Artificial Sequence
  Human GAPDH FW primer
ggagcgagatccctccaaaat

SEQ ID NO: 14 - DNA, 23 bp, Artificial Sequence
  Human GAPDH RV primer
ggctgttgtcatacttctcatcg

SEQ ID NO: 15 - DNA, 20 bp, Artificial Sequence
  HSV DNA FW
atcaacttcgactggccctt

SEQ ID NO: 16 - DNA, 20 bp, Artificial Sequence
  HSV DNA RV
ccgtacatgtcgatgttcac

SEQ ID NO: 17 - DNA, 20 bp, Artificial Sequence
  HSV FW
ctacatggcattggacagga

SEQ ID NO: 18 - DNA, 20 bp, Artificial Sequence
  HSV RV
ggtcacgttgccattcacta

(19) United States
(12) Patent Application Publication
(10) Pub. No.: US 2022/0154143 A1
(43) Pub. Date: May 19, 2022

(54) Culture Method for Head and Neck Organoids

(71) Applicant: Koninklijke Nederlandse Akademie Van Wetenschappen, Utrecht (NL)

(72) Inventors: Johannes Carolus Clevers, Utrecht (NL); Else Driehuis, Utrecht (NL)

(73) Assignee: Koninklijke Nederlandse Akademie Van Wetenschappen, Utrecht (NL)

(21) Appl. No.: 17/437,477

(22) PCT Filed: Mar. 16, 2020
(86) PCT No.: PCT/EP2020/057147

§ 371 Date: Sep. 9, 2021

(30) Foreign Application Priority Data
Mar. 15, 2019 (GB) 1903573.2

(51) Int. Cl. C12N 5/071; C12N 5/09; G01N 33/50

(52) U.S. Cl. CPC C12N 5/0625; C12N 5/0693; G01N 33/5073; C12N 2501/01; C12N 2501/02; C12N 2501/115; C12N 2500/32; C12N 2513/00; C12N 2503/04; C12N 2501/415; C12N 2501/999; C12N 2501/11; C12N 2500/38; C12N 2501/119

(57) ABSTRACT

The invention relates to in vitro cell culture methods for expanding epithelial cells from head and neck tissue, including head and neck tumour tissue, to obtain organoids. The invention relates to culture media suitable for use with said methods, organoids obtainable or obtained by said methods and uses of said culture methods, media and organoids in drug discovery and validation, toxicity assays, diagnostics and therapy.

### FIELD

The invention to relates to in vitro cell culture methods for expanding epithelial cells from head and neck tissue, including head and neck tumour tissue, to obtain organoids. The invention relates to culture media suitable for use with said methods, organoids obtainable or obtained by said methods and uses of said culture methods, media and organoids in drug discovery and validation, toxicity assays, diagnostics and therapy.

### BACKGROUND

The oral cavity, pharynx and larynx are lined by a stratified mucosa that protects the underlying structures. These epithelia are keratinizing or non-keratinizing, depending on the anatomical location (1). Neoplasia's commonly arise in this epithelium, with a worldwide incidence of over half a million patients a year (2). Well-known risk factors are alcohol and tobacco (3). Head and neck squamous cell carcinomas (HNSCCs) are difficult to treat, partly because of their anatomical location that complicates surgery, and partly due to the highly variable treatment response. Advanced cases require combinations of surgery, radiotherapy and chemotherapy. Taken together, this results in relapse rates of over 50% (4). Currently there are no reliable models to predict therapy outcome and guide treatment decisions.

In vitro studies of this epithelium have relied on tumour-derived 2D cell lines (5) and on primary keratinocyte cultures (6). Success rates to establish HNSCC-derived cell lines range from 11-33% (7). For primary keratinocyte cultures, keratinocytes are grown on feeder cells (mouse fibroblasts) in 2D and have a limited lifespan. In vitro drug screens of 2D lines have been used to characterize variability in drug response among tumours and as a tool to understand resistance mechanisms of tumour cells (8,9). In an attempt to overcome limitations of these 2D systems (10,11), HNSCC lines have been replated in a 3D format. When compared to 2D cell lines, these 3D models better recapitulate characteristics of in vivo HNSCC (12,13). In addition to in vitro models, HNSCC xenograft mouse models were introduced over thirty years ago (14). Lastly, transgenic mouse models have been developed to understand molecular drivers of HNSCC tumorigenesis (15). Although these models provided important insights in HNSCC, they are lacking the potential for a personalized approach.

In addition to malignant transformation, the oral mucosa is subject to viral infection (16). Herpes Simplex Virus (HSV) is amongst the most commonly encountered viral infection in the oral cavity (16). HSV is known to infect keratinocytes and give rise to herpes labialis (cold sores) (17). Infection with Human Papilloma Virus (HPV) is associated with oropharyngeal HNSCC and characterizes a genetically distinct subgroup of tumours with better prognosis than HPV-HNSCC (18). In vitro culture systems to study the interaction of keratinocytes with HPV are limited to the use of immortalized cell lines, or to primary cells that can only be cultured short-term (19).

Protocols to grow organoids from adult human tissues have been described for single-layered (simple) or (pseudo) stratified epithelia, such as those that line the colon, intestine, liver, pancreas, stomach, esophagus, prostate, lung, breast, and fallopian tube and cancers derived thereof (20). Tumour organoids have previously been shown to recapitulate the tumour from which they are derived, allowing in vitro drug responses to be linked to genetic alterations present in the original tumour (20). A recent study on gastrointestinal cancers documented a strong correlation response of the corresponding tumour organoids and patient clinical outcome (21).

The genetic alterations found in HNSCC are commonly found in other tumour types, and therapies targeting some of these specific mutations exist. Regardless, with the exception of Cetuximab (an anti-EGFR antibody) that is used in treatment of HNSCC, no targeted therapies are currently applied in standard care of these patients (41-44). In recent years, it has become clear that mutation status alone does not provide the required specificity or sensitivity to serve as a predictive marker (45).

Although culture media and methods have been described for obtaining organoids from epithelial stem cells, there is a need in the art for improved methods of generating organoids from epithelial stem cells derived from head and neck tissue.

Methods for culturing epithelial stem cells from a variety of tissues have previously been described (e.g. in WO2009/022907, WO2010/090513, WO2012/014076, WO2012/168930, WO2015/173425, WO2016/083613, and WO2016/083612). However, there is a need for improved methods for culturing head and neck tissue.

### SUMMARY OF THE INVENTION

Accordingly, the invention provides a method for culturing epithelial stem cells derived from head and neck tissue, wherein said method comprises culturing one or more epithelial stem cells in contact with an extracellular matrix in the presence of a culture medium as described herein.

In particular, the invention provides a method for culturing an epithelial stem cell or a population of epithelial stem cells derived from head or neck tissue, wherein the method comprises culturing one or more epithelial stem cells in contact with an extracellular matrix in the presence of an expansion medium, the expansion medium comprising a basal medium for animal or human cells and further comprising: one or more mitogenic growth factor, a TGF-beta inhibitor, an activator of the prostaglandin signalling pathway, one or more Wnt agonist, a cAMP pathway activator, a BMP inhibitor and nicotinamide.

The invention also provides an expansion medium comprising a basal medium for animal or human cells and further comprising: one or more mitogenic growth factor, a TGF-beta inhibitor, an activator of the prostaglandin signalling pathway, one or more Wnt agonist, a cAMP pathway activator, a BMP inhibitor and nicotinamide.

The invention also provides an organoid obtainable or obtained by said method.

The invention also provides a composition comprising said expansion medium and said organoid.

The invention also provides the use of said organoid as a model for viral infection.

The invention also provides said organoid for use in diagnostics or medicine, optionally in personalised medicine or diagnostics, or regenerative medicine.

The invention also provides a method of treating a disease comprising the step of administering said organoid.

The invention also provides the use of said organoid in the manufacture of a medicament for treating a disease.

The invention also provides the use of said organoid in drug screening, target validation, target discovery, toxicology, toxicology screens or an ex vivo cell/organ model.

The invention also provides said organoid for use in a method to guide personalised therapy.

The invention also provides the use of said organoid in an ex vivo method to predict a clinical outcome.

The invention also provides a method for testing the effect of a candidate compound, wherein the method comprises:
said method for culturing an epithelial stem cell or a population of epithelial stem cells derived from head or neck tissue;
exposing the resultant population of cells or the resultant organoid to one or a library of candidate compounds;
evaluating said expanded organoids for any effects,
identifying the candidate molecule that causes said effects as a potential drug; and optionally
providing said candidate molecule, e.g. as a drug.

The invention also provides a method for selecting a treatment regime for a patient, wherein the method comprises the steps of:
optionally obtaining a biopsy from the head or neck tissue of the patient;
culturing the biopsy, a tissue fragment of the biopsy, an epithelial stem cell of the biopsy or a population of epithelial stem cells of the biopsy according said method for culturing an epithelial stem cell or a population of epithelial stem cells derived from head or neck tissue to obtain an organoid or a tumour organoid;
exposing the resultant organoid to a treatment regime, including radiation and/or one or more candidate compounds;
evaluating said organoid for any effects;
identifying the treatment of regime that causes said effects; and
optionally providing said treatment to the patient.

### BRIEF DESCRIPTION OF THE DRAWINGS

FIG. 1. Oral mucosa organoids can be established from mouse tongue epithelium. A. brightfield microscopy images and H&E staining of paraffin-embedded organoid sections of organoids established from different regions of the mouse tongue (annotated 1, 2 and 3, see schematic in B) B. Organoids keratinize at larger sized, revealed by darker centers in the brightfield images or acellular parts in the H&E staining. Scalebar top panels 100 μm, scalebar bottom panels 500 μm. B. Schematic of locations annotated 1, 2 and 3 in FIG. 1A.

FIG. 2. Organoids can be derived from healthy oral mucosa and recapitulate morphological and functional characteristics. A. Schematic outline of the digestion and initial culture condition of oral mucosa organoids. If the patient signs informed consent, tissue that is obtained via biopsy or resection is collected, digested using trypsin and subsequently plated. Over time, organoids grow out from the primary tissue. B. Brightfield microscopy image of an organoid lines derived from oral mucosa epithelium, scalebar 500 μm. C. Total cell numbers obtained from organoid cultures over the course of 6 weeks. Cell numbers were determined in two independently established normal oral mucosa organoid lines (N1 and N10). Counting was performed in quadruplicate. Quantification shows stable growth of the organoid lines. D. Hematoxylin and eosin (H&E) staining and immunostaining for MKi67, TP63 and oral mucosa specific KRT13 of paraffin-embedded organoids and control tissue. As can be seen, proliferating basal cells (Ki67 and p63 positive) reside in the periphery of the organoids, while more differentiated keratinocytes (KRT13 positive) reside in the centre of the organoid. Scalebar 100 μm. E. Quantitative PCR of a normal oral mucosa organoid line (N5) for proliferation marker MKI67, basal cell marker TP63 and KRT13, Prior to RNA collection, growth factors were withdrawn from the medium to induce differentiation. Expression levels are calculated using ΔΔCt method. For each marker, fold change in expression is shown relative to expression of this markers in human primary tongue tissue, which is set to 1. n=3, individual data points are shown, bars represent average. F. Number of chromosomes was determined for N8 organoids in early passage (p8) and later passage (p16) by metaphase spread analysis.

FIG. 3. Outgrowth of human oral mucosa organoids and characterization using scanning electron microscopy and transmission electron microscopy, related to FIG. 2. A. Organoid outgrowth can be observed from human primary tissue when put in culture. Representative images of establishment of an organoid culture. Starting one day after initial plating of the tissue, images of the same BME drop with human cells were taken on day 1, 2, 5, 6 and 7 to show outgrowth of organoids from primary tissue. Scalebar 500 μm. B. Scanning electron microscopy of human oral mucosa organoids. First panel: an organoid that broke open during processing shows the apical surface of organoid cells. Most cells have a smooth surface, whereas some cells have a folded apical surface. Second panel: zoom in of the apical surface of an organoid, showing multiple keratinocytes forming tight connections. Scalebar 10 μm C. Transmission electron microscopy images of human oral mucosa organoids. First panel: a single keratinocyte shows properties characteristic for keratinocytes, such as abundant tonofilament formation (asterixes) and tight junctions (arrows) connecting it to neighbouring cells. Second panel: cross section spanning the apical part of the organoid wall. Cells located more towards the outside of the organoids are bigger, more rounded and have intact nuclei. Moving more towards the inside of the organoid, cells seem to flatten out, and lose their nucleus. Third panel, cross section showing the inside of an organoid, where cell fragments are still present. One keratinocyte is being shed into the inside of the structure. Scalebars are shown below each individual panel. E. Quantitative PCR of a normal oral mucosa organoid line (N8) for proliferation marker MKI67, basal cell marker TP63 and KRT13, Prior to RNA collection, growth factors were withdrawn from the medium to induce differentiation. Expression levels are calculated using ΔΔCt method. For each marker, fold change in expression is made relative to expression of this markers in human primary tongue tissue, which is set to 1. n=3, individual data points are shown, bars represent average.

FIG. 4. Oral mucosa organoids can be productively infected with Herpes Simplex Virus. A. Immunohistochemical staining for dTomato was performed on organoids infected with dTomato labelled HSV (dTOM-HSV). Organoids were collected on day 0, 1, 2 and 3. Scalebar 50 μm. B. Live cell imaging of dTOM-HSV infected organoids. Two organoids were followed over time and pictures of the following timepoints are depicted in this figure: t=0, 24, 39, 46 and 60 hours, scalebar 100 μm. C. Immunohistochemical staining for dTomato performed on paraffin-embedded organoids that were infected with HSV-dTomato and maintained in culture for two weeks, scalebar 100 μm. D. Quantitative PCR of DNA obtained from oral mucosa organoids infected with HSV and kept in culture for 10 days. HSV can replicate in oral mucosa organoids, and this replication can be inhibited by the addition of acyclovir. Fold increase in DNA content is shown relative to uninfected control at day 0. Data points represent the average of three technical replicates, error bars represent the SEM. Dark line, organoids infected with HSV. Light line, organoids infected with HSV and cultured in the presence of 1 μM acyclovir. Darkest line, organoids not infected with HSV. E and F. Quantitative PCR for HPV on DNA obtained from oral mucosa organoids infected with HPV (E) or HPV-conditioned medium (F) and kept in culture for a maximum 10 days. Fold increase in DNA content is shown relative to uninfected control at day 0. Data points represent the average of three technical replicates, error bars represent the SEM.

FIG. 5. HNSCC organoids can be established and recapitulate functional 969 and morphological characteristics of the tumour. A. Overview of the tumours of which organoids were established in this study and their anatomical location. B. Hematoxylin and eosin (H&E) staining and immunostaining for basal cell marker TP40, tumour suppressor TP53, proliferation marker MKI67 and KRT5 of paraffin-embedded organoids and corresponding tissue. Scalebar, 100 μm C. Organoids established from HNSCC and corresponding normal tissue of the same patient show a different response to Mdm2 agonist Nutlin-3. Scalebar 500 μm. D. H&E and immunostaining for TP53 performed on sections of paraffin-embedded organoids reveal differences in morphology and p53 status of the two organoid lines. Scalebar 100 μm. E. Principal component analysis of RNA sequencing data of 6 normal wildtype (darker) and 6 tumour organoid lines (lighter). Two tumour samples were sequenced in two independent runs as a quality control. These samples cluster together. Tumour-derived organoid cluster together and away from the normal wildtype-epithelium derived organoids. There are two exceptions to this clustering as N10 and T6 do not cluster together with the other normal- and tumour derived organoid lines, respectively. F. Heatmap depicting expression of the 51 differentially expressed genes between normal- and tumour-derived organoids (p<0.01) in the sequenced organoids. Differential expression was calculated as described in DESeq2 package (30). Genes marked in grey are described in the text and were reported by others to be differential expressed in HNSCC. For these genes, FIG. 9 shows the expression values relative to normal organoids.

FIG. 6. Brightfield images of HNSCC-derived organoid lines, related to FIG. 5. For all organoid lines characterized in this work, images are shown of organoids in culture. Scalebar, 500 μm.

FIG. 7. H&E staining of HNSCC-derived organoids reveals differences in morphology between different organoid lines, related to FIG. 5. H&E staining performed on sections of paraffin-embedded organoids. Here, H&E staining of four normal and eight tumour lines are shown.

FIG. 8. Normal and tumour organoids derived from the same patient show different morphology, related to FIG. 5. Organoids were derived from both tumour tissue and adjacent normal tissue from the same patient. H&E staining of organoids are shown and reveal different morphology of the two organoid lines.

FIG. 9. HNSCC-derived organoid show differences in sensitivity to Nutlin-3, related to FIG. 5. Organoids were cultured for three passages in the presence of 10 μM Nutlin-3 and passaged weekly. Left panels, organoid cultured in the absence of Nutlin-3. Right panels, organoids cultured in the presence of Nutlin-3. All tumour lines, except T8, T9 and T10 are resistant to these compounds. Both normal lines (N1 and N5, corresponding normal organoids of T1 and T5) show Nutlin-3 sensitivity. Scalebar, 500 μm.

FIG. 10. Expression analysis of normal versus tumour organoids, related to FIG. 5. A. Heatmap of expression of the seven genes found significantly differentially expressed upon DEseq2 analysis comparing normal and tumour organoids. B. Scatterplots of the expression of these seven genes, plotted for each individual gene.

FIG. 11. HNSCC-derived organoids recapitulate genetic alterations found in this tumour type. A. Mutations detected in HNSCC-derived organoids T1 to T10. The colour of the square indicates the type of mutation detected. Colour intensities indicate the variant allele frequency (VAF) of the detected genetic alteration. Anatomical source of the organoid line is shown. B. Circos plot showing the single nucleotide variants (SNV, outer track) and small insertions or deletions (Indels, inner track) for T5 tissue (outer circle), T5 organoids (middle circle) and N5 organoids (inner circle). All variants are relative to N5 tissue.

FIG. 12. Comparison of sequencing results of primary tissue and organoid cultures of patient 3 and 5, related to FIG. 11. Percentages of reads carrying detected mutation upon targeted sequencing performed in both primary tissue and organoids from patient 3 and 5.

FIG. 13. HNSCC-derived organoids are chromosomally unstable in vitro. Imaging of H2B mNEON expressing organoids reveals cell divisions that can be studied and quantified for segregation errors during division. A. Quantification of segregation errors observed in N1 and T1. Percentage of mitotic errors per organoids is shown. One dot represents an imaged organoid and the colour of the dot indicates the corresponding number of mitoses. B-D. Stills taken from time-lapse movies of N1 and T1 organoids. Maximum projections of selected Z-planes are shown. B Examples of a correct mitosis observed in N1. C. Example of an anaphase bridge formed during mitosis in T1. D. Example of a bi-nucleated cell undergoing multipolar division observed in T1. Scalebar 20 μm. E. Scatter plot, presenting chromosome number distribution and median, based on organoid metaphase spreads of N1 and T1 cells. F. Scatter plot, presenting chromosome number distribution and median, based on organoid metaphase spreads of T1 to T9 tumour organoid cells.

FIG. 14. Xenografted HNSCC organoids recapitulate histopathological characteristics of HNSCCs. A. Three independent mice were injected with each organoid line, and the number of mice that developed tumours is depicted. B. Hematoxylin and eosin (H&E) and anti-human nuclei staining of the paraffin-embedded tumour. Left panel, H&E reveals stratification from more basal (dark purple) to more differentiated keratinocytes (light purple) to eventually deposited keratin (pink). Right panel, immunostaining for human nuclei reveals human origin of the squamous epithelial cells observed in the tumour. C. Comparison of two tumours originating from either organoid line T2 or T6. Left panels, H&E reveal different morphology of the different tumours. Right panels, Ki-67 staining shows difference in the number of cells in G1 between the two tumours. D. Example of atypia that can be observed in the tumours. Arrow indicates a tripolar mitotic figure. Throughout the image, nuclear pleomorphism can be observed. E. Squamous cells can invade into the surrounding muscle tissue of the mouse. Striped scalebar, 1000 μm. Solid scalebar, 100 μm.

FIG. 15. HNSCC-derived organoids result in tumour formation in vivo, related to FIG. 14. For all transplanted organoid line, three mice were injected. Here, H&E and anti-human nuclei staining for these tumours is shown. As can be seen, histology of tumours originating from the same organoid line matches. Scale bar, 100 μm.

FIG. 16. HNSCC organoids as a platform for drug screening. Validation of the drug screen set-up using Nutlin-3 exposure. Drug screen viability is consistent with the Nutlin-3 response observed using brightfield imaging. Scalebar 500 μm. B. Heatmap showing the tumour organoids ranked based on Nutlin-3 IC50. IC50 values and TP53 mutation status are depicted. C. Heatmap showing the organoid lines ranked based on cisplatin and carboplatin sensitivity. D. Correlation between cisplatin sensitivity (x-axis) and carboplatin sensitivity (y-axis) can be observed in vitro, Pearson correlation, r=0.71, p<0.05. Each dot represents one tumour organoid line, for which the cisplatin IC50 value is plotted on the x-axis, and the carboplatin IC50 value is plotted on the y-axis. E. Heatmap showing the organoid lines ranked based on Cetuximab sensitivity as measured by area under the curve (AUC). Here, AUC was used instead of IC50 values, because of the curvature of the kill curve, that did not allow for IC50 value calculation. Red indicates low AUC values, blue indicates high AUC values. In this dataset, no correlation between EGFR expression and Cetuximab sensitivity can be observed. Mutations in downstream components of EGFR (PIK3CA, KRAS, NRAS and BRAF) confer resistance to Cetuximab. F. Organoids show variable sensitivity to radiation. Cell viability is plotted on the y-axis, for different amounts of radiation, ranging from 0 to 10 Gy (x-axis). G and H. Chemoradiation therapy of tumour organoids. Cisplatin and Cetuximab screens were performed either in the presence (‘chemo+RT (no RT=100%)’) or absence (‘chemo’) of radiation. Viability was calculated relative to untreated (no chemo, no RT) organoids, for which viability was set to 100%. To depict the effect of chemotherapy only, in the presence of RT, viability of the radiated tumour organoids was calculated relative to tumour organoids that were exposed to radiation, but no chemotherapy. This result is depicted by the line ‘chemo+RT (RT only=100%)’. This line shows the effect of chemotherapy in the presence of RT, corrected for the effect of RT only, whereas the ‘chemo+RT (no RT=100%)’ line shows the overall effect of this combination therapy on tumour organoid viability, compared to no treatment. I. Sensitivity of tumour organoid lines T1, T7 and T9 for PI3K-inhibitor Alpelisib. T9, carrying a H1047R activating PIK3CA shows increased sensitivity to this agent, whereas T1 and T7, both carrying an E545K PIK3CA mutation, do not show this. IC50 values are shown in the top right of the graph. J. Sensitivity of tumour organoid lines T4, T5, T8 and T9 for BRAF-inhibitor Vemurafenib. T9 (carrying a BRAF V600E activating mutation) shows increased sensitivity to this agent. IC50 values are shown in the top right of the graph.

FIG. 17. In vitro drug screens in HNSCC-derived organoids, related to FIG. 16. A. Schematic layout of the drug screens as performed in this study. Organoids were disrupted into single cells on day 0, and plated to recover for two days. On day 2, organoids were collected from the BME, washed, filtered, counted and plated in 5% BME in organoid medium in 384 well format (500 organoids per well). Subsequently a gradient of drug concentrations was printed in the wells (different drugs are represented by different colours in the figure), and cells were left exposed to the drugs for five days. As positive control, cells were exposed to 1 μM staurosporin. Solvent volumes were normalized for each plate, so that percentage DMSO or PBS/Tween-20 was identical for each well. Wells with only normalization were used as negative control. Each drug concentration was tested in triplicate. Readout was performed using Cell Titer Glow. B. To assess the reproducibility of the assay, the same drug screen was performed three times (technical replicates, named as TR1, TR2 and TR3). Calculated viability for each individual data point was plotted against its replicate value to assess robustness of the assay. C. Z factor scores of the performed drug screens for all drugs and all organoid lines presented in this work. D. In vitro radiation sensitivity screen. As an example, brightfield images on the day of readout are shown here for T3 and T4. T3 is more sensitive to radiation when compared to T4, which can be seen also from the number and the size of the organoids when compared to organoids that were not exposed to radiation. Scale bar 500 μm.

FIG. 18. Sensitivity of HNSCC-derived organoids exposed to all compounds used in this study, related to FIG. 16. Drugscreen results from T1 t/m T9 exposed to cisplatin, carboplatin, cetuximab, AZD4547, everolimus, Alpelisib, niraparib and vemurafenib.

FIG. 19. Patient clinical data.

FIG. 20. DeSeq2 results of HNSCC-derived organoids versus normal wildtype control organoids.

FIG. 21. Detected mutations/CNV in T1 to T11 in genes checked in the OncoAMP panel.

FIG. 22. Optimised culture medium for culturing established head and neck organoids. Established head and neck organoids derived from normal tissue were cultured in the medium described in Table 1. For the conditions indicated as “terminated”, the medium was not able to support the culture of head and neck organoids.

FIG. 23. Optimised culture medium for culturing head and neck organoids from primary tissue. A. Primary tissue was cultured in the medium described in Table 1 to generate head and neck organoids. For the conditions indicated as “terminated”, the medium was not able to support the culture of head and neck organoids. B. Brightfield images of the cultures described in A.

### DETAILED DESCRIPTION

The methods of the invention allow head and neck organoids to be obtained by expanding epithelial stem cells. The methods allow exponential growth and long-term expansion. This enables a large number of cells to be available for various applications, for example, drug screening, in which a large amount of material is required to test various different drugs. The ability to generate the cells from a single starting source is advantageous for such applications where it is necessary to compare results between experiments. Similarly, it means that many cells are available for use in transplants and that multiple patients may be transplanted with cells obtained from a useful donor.

Culturing the cells in an expansion medium allows the cells to multiply whilst retaining their stem cell phenotype. Organoids are formed comprising these stem cells. Use of the expansion medium is therefore advantageous for providing increased numbers of these useful stem cells and for obtaining organoids containing these cells.

Accordingly, there is provided a method for culturing epithelial stem cells derived from head or neck tissue, wherein said method comprises culturing one or more epithelial stem cells in contact with an extracellular matrix in the presence of an expansion medium, the expansion medium comprising a basal medium for animal or human cells and further comprising one or more mitogenic growth factors, a TGF-beta inhibitor, an activator of the prostaglandin signalling pathway, one or more Wnt agonists, a cAMP pathway activator, a BMP inhibitor and nicotinamide.

There is also provided an expansion medium comprising a basal medium for animal or human cells, and further comprising one or more mitogenic growth factors, a TGF-beta inhibitor, an activator of the prostaglandin signalling pathway, one or more Wnt agonists, a cAMP pathway activator, a BMP inhibitor and nicotinamide.

### Mitogenic Growth Factors

The expansion medium of the invention comprises a mitogenic growth factor. Mitogenic growth factors typically induce cell division via the mitogen-activated protein kinase signalling pathway. Many receptor tyrosine kinase ligands are mitogenic growth factors. In some embodiments, the mitogenic growth factor can bind to a receptor tyrosine kinase. In some embodiments, the mitogenic growth factor can bind to more than one receptor tyrosine kinase. In some embodiments, the one or more mitogenic growth factor binds to a receptor tyrosine kinase such as EGFR, an FGFR or HGFR, optionally wherein the one or more mitogenic growth factors are selected from EGF, FGF and HGF.

Epidermal growth factor receptor (EGFR), also known as ErbB1 or HER1, is a cell surface receptor for members of the epidermal growth factor (EGF) family of extracellular protein ligands. EGFR is a receptor tyrosine kinase and belongs to the HER family of receptors which comprise four related proteins (EGFR (HER1/ErbB1), ErbB2 (HER2), ErbB3 (HER3) and ErbB4 (HER4)). The HER receptors are known to be activated by binding to different ligands, including EGF, TGF-alpha, heparin-binding EGF like growth factor, neuregulin, amphiregulin, betacellulin, and epiregulin. After a ligand binds to the extracellular domain of the receptor, the receptor forms functionally active dimers (EGFR-EGFR (homodimer) or EGFR-HER2, EGFR-HER3, EGFR-HER4 (heterodimer)). Dimerization induces the activation of the tyrosine kinase domain, which leads to autophosphorylation of the receptor on multiple tyrosine residues. This leads to recruitment of a range of adaptor proteins (such as SHC, GRB2) and activates a series of intracellular signalling cascades to affect gene transcription. Therefore, in some embodiments, the mitogenic growth factor binds to EGFR, HER1, HER2, HER3 or HER4. In some embodiments, the mitogenic growth factor binds to EGFR. In some embodiments, a HER2-4 ligand is included in the culture medium in addition to an EGFR ligand. For example, in some embodiments, neuregulin is included in the culture medium in addition to EGF. Neuregulin has been shown to be advantageous for culture of lung and breast tissue (e.g. see WO2016/083613, and WO2016/083612). In some embodiments, the one or more mitogenic growth factor in the expansion medium is EGF. Any suitable EGF may be used, for example, EGF obtained from Peprotech. EGF is preferably added to the basal culture medium at a final concentration of between 0.1 ng/ml and 500 ng/ml, between 0.1 ng/ml and 400 ng/ml, between 0.1 ng/ml and 300 ng/ml, between 0.1 ng/ml and 200 ng/ml, between 0.1 ng/ml and 100 ng/ml, between 1 ng/ml and 100 ng/ml, or wherein the final concentration of a mitogenic growth factor is approximately 1 ng/ml, 2 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 250 ng/ml or 500 ng/ml and not higher than 500 ng/ml. A more preferred concentration is at least 50 and not higher than 100 ng/ml. An even more preferred concentration is about 50 ng/ml. FGFs stimulate cells by interacting with cell surface tyrosine kinase receptors (FGFR). Four closely related receptors (FGFR1-FGFR4) have been identified. Therefore, in some embodiments, the mitogenic growth factor binds to an FGF receptor family member. FGF receptor family members include (but are not limited to) FGFR1, FGFR2, FGFR3 or FGFR4. FGFR1-FGFR3 genes have been shown to encode multiple isoforms, and these isoforms can be critical in determining ligand specificity. There are several FGFs that bind to the FGF receptor family members, including (but not limited to) FGF2, FGF4, FGF7 and FGF10. These are commercially available. Therefore, in some embodiments, the mitogenic growth factor is an FGF. In some embodiments, the FGF is selected from FGF2, FGF4, FGF7 and FGF10. In preferred embodiments, the FGF is FGF2 and/or FGF10. In a most preferred embodiment, the FGF is FGF2 and FGF10.

Most FGFs bind more than one receptor (Ornitz J Biol Chem. Feb. 27, 1998; 273 (9):5349-57). However, FGF10 and FGF7 are unique among FGFs in that they interact only with a specific isoform of FGFR2, designated FGFR2b which is expressed exclusively by epithelial cells (Igarashi, J Biol Chem. 1998 273(21):13230-5). Therefore, in some embodiments the mitogenic growth factor binds to FGFR2b. FGF10 has been shown to be particularly useful in the expansion medium. FGF10 is able to bind to FGFR2 or FGFR4. Therefore, in some embodiments, the mitogenic growth factor binds to FGFR2 or FGFR4. The presence of FGF2 in the expansion medium is particularly advantageous for culturing epithelial stem cells and organoids derived from head and neck tissue. FGF2 binds to all of FGFR1, FGFR2, FGFR3 and FGFR4. Therefore, in some embodiments, the mitogenic growth factor binds to all of FGFR1, FGFR2, FGFR3 and FGFR4.

In some embodiments, the final concentration of FGF is between 0.1 ng/ml and 500 ng/ml, between 0.1 ng/ml and 400 ng/ml, between 0.1 ng/ml and 300 ng/ml, between 0.1 ng/ml and 200 ng/ml, between 0.1 ng/ml and 100 ng/ml, between 1 ng/ml and 100 ng/ml, or wherein the final concentration of a mitogenic growth factor is about 1 ng/ml, 2 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 250 ng/ml or 500 ng/ml.

In some embodiments, the one or more mitogenic growth factor in the expansion medium is FGF10. Preferred concentrations for FGF10 are approximately between 0.1 ng/ml and 500 ng/ml, between 0.1 ng/ml and 400 ng/ml, between 0.1 ng/ml and 300 ng/ml, between 0.1 ng/ml and 200 ng/ml, between 0.1 ng/ml and 100 ng/ml, between 1 ng/ml and 100 ng/ml, or wherein the final concentration of a mitogenic growth factor is approximately 1 ng/ml, 2 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 250 ng/ml or 500 ng/ml and not higher than 500 ng/ml. A more preferred concentration for FGF10 is about 10 ng/ml.

In some embodiments, the one or more mitogenic growth factor in the expansion medium is FGF2. Preferred concentrations for FGF2 are approximately between 0.1 ng/ml and 500 ng/ml, between 0.1 ng/ml and 400 ng/ml, between 0.1 ng/ml and 300 ng/ml, between 0.1 ng/ml and 200 ng/ml, between 0.1 ng/ml and 100 ng/ml, between 1 ng/ml and 100 ng/ml, or wherein the final concentration of a mitogenic growth factor is approximately 1 ng/ml, 2 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 250 ng/ml or 500 ng/ml and not higher than 500 ng/ml. A more preferred concentration for FGF2 is about 5 ng/ml.

Hepatocyte growth factor/scatter factor (HGF/SF) is a morphogenic factor that regulates cell growth, cell motility, and morphogenesis by activating a tyrosine kinase signalling cascade after binding to the proto-oncogenic HGFR. The HGFR is also known as the c-Met receptor. HGF has been shown to be useful in epithelial stem cell culture. Therefore, in some embodiments the mitogenic growth factor binds HGFR. In some embodiments, the mitogenic growth factor is HGF. Any suitable HGF may be used, for example, HGF obtained from Peprotech. Preferred concentrations for HGF are about 1, 10, 20, 25, 50 ng/ml, not higher than 50 ng/ml.

In some embodiments, more than one mitogenic growth factor is included in the expansion medium, e.g. two or three mitogenic growth factors. For example, in some embodiments, the one or more mitogenic growth factors in the expansion medium are EGF and FGF. In some embodiments, the one or more mitogenic growth factors in the expansion medium are EGF, FGF2 and FGF10. In some embodiments, the one or more mitogenic growth factors in the expansion medium are EGF, optionally at a final concentration of about 50 ng/ml, FGF2, optionally at a final concentration of about 5 ng/ml, and FGF10, optionally at a final concentration of about 10 ng/ml.

In some embodiments hepatocyte growth factor (HGF) is also present in the presence or absence of EGF and/or FGF.

In some embodiments, the final concentration of each mitogenic growth factor is between 0.1 ng/ml and 500 ng/ml, between 0.1 ng/ml and 400 ng/ml, between 0.1 ng/ml and 300 ng/ml, between 0.1 ng/ml and 200 ng/ml, between 0.1 ng/ml and 100 ng/ml, between 1 ng/ml and 100 ng/ml, or wherein the final concentration of a mitogenic growth factor is about 1 ng/ml, 2 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 250 ng/ml or 500 ng/ml.

### Wnt Agonist

The expansion medium of the invention comprises one or more Wnt agonist. A Wnt agonist is defined herein as an agent that activates or enhances TCF/LEF-mediated transcription in a cell.

The canonical Wnt signalling pathway is defined by a series of events that occur when the cell-surface Wnt receptor complex, comprising a Frizzled (FZD) receptor and LRP is activated, usually by an extracellular signalling molecule, such as a member of the Wnt family of secreted glycoproteins. This results in the activation of Dishevelled family proteins which inhibit a complex of proteins that includes axin, GSK-3, and the protein APC to degrade intracellular β-catenin. The resulting enriched nuclear β-catenin enhances transcription by TCF/LEF family transcription factors (Driehuis & Clevers, British Journal of Pharmacology (2017) 174 4547-4563).

The R-spondin/Rnf43/Lgr module further regulates canonical Wnt signalling. In the absence of R-spondin, E3 ligases RNF43/ZNRF3 ubiquinate FZD, thus marking it for degradation by the proteasome and inhibiting Wnt signalling. When extracellular R-spondin is present, it can interact with membrane-spanning E3 ligases RNF43/ZNRF3 via an Lgr receptor, preventing the action of the E3 ligases. Lgr receptors, including Lgr4, Lgr5 and Lgr6, but particularly Lgr5, are expressed on epithelial stem cells. R-spondin is recruited by these stem cell markers to enhance Wnt signalling, and thus R-spondin and Lgr interact to promote proliferation and retain the stem cell multipotency. For these reasons, R-spondin family proteins have been shown to be particularly useful in epithelial stem cell culture for obtaining long-lived organoid cultures.

The one or more Wnt agonist in the expansion medium may therefore be selected from a Wnt ligand from the Wnt family of secreted glycoproteins, an inhibitor of intracellular β-catenin degradation, a GSK-3 inhibitor, activators of TCF/LEF, an inhibitor of RNF43 or ZNRF3, and R-spondin family proteins. In some embodiments, the Wnt agonist in the expansion medium comprises an R-spondin family protein and a GSK-3 inhibitor, and optionally further comprises a Wnt ligand from the Wnt family of secreted glycoproteins.

The R-spondin family protein (also referred to herein as “R-spondin”) may be selected from R-spondin 1, R-spondin 2, R-spondin 3, R-spondin 4 and analogs, fragments, variants and derivatives thereof. In this context, the fragment, variant or derivative is capable of preventing the action of the E3 ligases RNF43/ZNRF3 on the Wnt receptor complex. R-spondin 1, R-spondin 2, R-spondin 3 and R-spondin 4 (also referred to herein as “R-spondin 1-4”) are all characterized by two amino-terminal furin-like repeats, which are necessary and sufficient for Wnt signal potentiation, and a thrombospondin domain situated more towards the carboxyl terminus members (Lau et al. Genome Biol. 2012; 13(3):242 (2012). Examples of R-spondin fragments, variants and derivatives suitable for use in the invention are known to the skilled person (e.g. see Example 2 of WO 2012/140274, which describes furin domain fragments which are capable of enhancing Wnt signalling and which are incorporated herein by reference). Examples of R-spondin family protein analogs include, for example, antibodies that interact with RNF43/ZNRF3/Lgr. Agonistic anti-Lgr5 antibodies that can enhance Wnt signalling are known in the art (e.g. see antibody 1 D9 described in Example 3 of WO 2012/140274).

Many GSK-3 inhibitors are known in the art (e.g. see Greengard, P., and Meijer, L. (2004) Structural basis for the synthesis of indirubins as potent and selective inhibitors of glycogen synthase kinase-3 and cyclin-dependent kinases. J Med Chem 47: 935-946; and Thomas Kramer, Boris Schmidt, and Fabio Lo Monte, “Small-Molecule Inhibitors of GSK-3: Structural Insights and Their Application to Alzheimer's Disease Models,” International Journal of Alzheimer's Disease, vol. 2012, Article ID 381029, 32 pages, 2012. https://doi.org/10.1155/2012/381029) and are available commercially (e.g. see the list available from Santa Cruz Biotechnology here: https://www.scbt.com/scbt/browse/GSK-3-beta-Inhibitors/_/N-x6oud). Any of these GSK-3 inhibitors are suitable for use in the context of the invention and the skilled person would be able to determine a suitable concentration using IC50 values and the teachings in the Example section of this application.

CHIR-99021 (CAS: 252917-06-9; 6-[[2-[[4-(2,4-Dichlorophenyl)-5-(5-methyl-1H-imidazol-2-yl)-2-pyrimidinyl]amino]ethyl]amino]-3-pyridinecarbonitrile; CT99021) is a potent and selective inhibitor of GSK-3 and has been shown to be particularly useful for head and neck organoid expansion (GSK3β, 1050=5 nM; and GSK3α IC50=10 nM). Other aminopyrimidine inhibitors with an IC50 value of 0.6 nM to 7 nM include CHIR98014 (Axon, Cat 1126), CHIR98023, CHIR99021 (see above), TWS119 (Tocris, Cat 3835). Therefore, in some embodiments, the GSK-3 inhibitor is an aminopyrimidine inhibitor, optionally selected from CHIR98014, CHIR98023, CHIR99021 or TWS119. In some embodiments the GSK-3 inhibitor is CHIR-99021.

Any suitable concentration of a GSK-3 inhibitor (e.g. CHIR-99021) may be used, for example, between 10 nM and 500 μM, between 10 nM, and 400 μM, between 10 nM and 300 μM, between 10 nM and 200 μM, between 10 nM and 100 μM, between 20 nM and 50 μM or between 100 nM and 5 μM, or wherein the final concentration is about 3 μM or wherein the final concentration is about 0.3 μM.

The Wnt ligand from the Wnt family of secreted glycoproteins may be selected from Wnt-I/Int-1, Wnt-2/Irp (InM-related Protein), Wnt-2b/13, Wnt-3/Int-4, Wnt-3a (R&D systems), Wnt-4, Wnt-5a, Wnt-5b, Wnt-6 (Kirikoshi H et al 2001 Biochem Biophys Res Com 283 798-805), Wnt-7a (R&D systems), Wnt-7b, Wnt-8a/8d, Wnt-8b, Wnt-9a/14, Wnt-9b/14b/15, Wnt-10a, Wnt-10b/12, WnM I, and Wnt-16. An overview of human Wnt proteins is provided in “THE WNT FAMILY OF SECRETED PROTEINS”, R&D Systems Catalog, 2004. In some embodiments, the Wnt ligand is Wnt-3a, Wnt-5 or Wnt-6a, or optionally is Wnt-3a. Addition of a soluble Wnt ligand has been shown to be particularly advantageous for expansion of human epithelial stem cells (e.g. as described in WO2012/168930).

The Wnt agonist is preferably added to the media in an amount effective to stimulate a Wnt activity in a cell by at least 10%, more preferred at least 20%, more preferred at least 30%, more preferred at least 50%, more preferred at least 70%, more preferred at least 90%, more preferred at least 100%, relative to a level of said Wnt activity in the absence of said molecule, as assessed in the same cell type. As is known to a skilled person, Wnt activity can be determined by measuring the transcriptional activity of Wnt, for example by pTOPFLASH and pFOPFLASH Tcf luciferase reporter constructs (Korinek et al., 1997. Science 275:1784-1787).

A soluble Wnt agonist, such as Wnt-3a, may be provided in the form of Wnt conditioned medium. For example, about 10% to about 30%, e.g. about 10 ng/ml to about 10 μg/ml, preferably about 1 μg/ml, Wnt conditioned medium may be used.

R-spondin may be provided in the form of R-spondin conditioned medium or in the form of recombinant protein. For example, about 10 ng/ml to about 500 ng/ml, about 10 ng/ml to about 400 ng/ml, about 10 ng/ml to about 300 ng/ml, about 10 ng/ml to about 250 ng/ml, about 50 ng/ml to about 250 ng/ml, about 100 ng/ml to about 250 ng/ml, or about 150 ng/ml to about 250 ng/ml, preferably about 150 ng/ml to about 250 ng/ml, R-spondin may be used. For example, the final concentration of R-spondin in the expansion medium may be about 10 ng/ml, about 25 ng/ml, about 50 ng/ml, about 75 ng/ml, about 100 ng/ml, about 125 ng/ml, about 150 ng/ml, about 175 ng/ml, about 200 ng/ml, about 225 ng/ml, about 250 ng/ml, about 275 ng/ml, about 300 ng/ml, about 325 ng/ml, about 350 ng/ml, about 375 ng/ml, about 400 ng/ml, about 450 ng/ml, or about 500 ng/ml.

One or more, for example, 2, 3, 4 or more Wnt agonists may be used in the expansion medium.

### TGF-Beta Inhibitor

The expansion medium comprises a TGF-beta inhibitor. The presence of a TGF-beta inhibitor in the expansion media is particularly advantageous for increasing human organoid formation efficiency. TGF-beta signalling typically begins with binding of a TGF-beta superfamily ligand to a type II receptor which recruits and phosphorylates a type I receptor. The type I receptor then phosphorylates SMADs, which act as transcription factors in the nucleus and regulate target gene expression.

The TGF-beta superfamily ligands comprise bone morphogenic proteins (BMPs), growth and differentiation factors (GDFs), anti-müllerian hormone (AMH), activin, nodal and TGF-betas. In general, Smad2 and Smad3 are phosphorylated by the ALK4, 5 and 7 receptors in the TGF-beta/activin pathway. By contrast, Smad1, Smad5 and Smad8 are phosphorylated as part of the bone morphogenetic protein (BMP) pathway. However, in the context of this invention, this skilled person will understand that a “TGF-beta inhibitor” or an “inhibitor of TGF-beta signalling” is an inhibitor of the TGF-beta pathway which involves ALK4, 5 and 7, and which involves Smad2 and Smad3. The TGF-beta inhibitor is not a BMP inhibitor, i.e. the skilled person would understand that noggin is not a TGF-beta inhibitor in the context of this disclosure. In some embodiments, a BMP inhibitor is added to the culture medium in addition to the TGF-beta inhibitor (see below).

Thus the TGF-beta inhibitor is any agent that reduces the activity of the TGF-beta signalling pathway, also referred to herein as the ALK4, ALK5 or ALK7 signalling pathway. There are many ways of disrupting the TGF-beta signalling pathway that are known in the art and that can be used in conjunction with this invention. For example, the TGF-beta signalling may be disrupted by: inhibition of TGF-beta expression by a small-interfering RNA strategy; inhibition of furin (a TGF-beta activating protease); inhibition of the pathway by physiological inhibitors; neutralisation of TGF-beta with a monoclonal antibody; inhibition with small-molecule inhibitors of TGF-beta receptor kinase 1 (also known as activin receptor-like kinase, ALK5), ALK4, ALK7; inhibition of Smad 2 and Smad 3 signalling, e.g. by overexpression of their physiological inhibitor, Smad 7, or by using thioredoxin as an Smad anchor disabling Smad from activation (Fuchs, O. Inhibition of TGF-Signaling for the Treatment of Tumor Metastasis and Fibrotic Diseases. Current Signal Transduction Therapy, Volume 6, Number 1, January 2011, pp. 29-43(15)).

Various methods for determining if a substance is a TGF-beta inhibitor are known and might be used in conjunction with the invention. For example, a cellular assay may be used in which cells are stably transfected with a reporter construct comprising the human PAI-1 promoter or Smad binding sites, driving a luciferase reporter gene. Inhibition of luciferase activity relative to control groups can be used as a measure of compound activity (De Gouville et al., Br J Pharmacol. 2005 May; 145(2): 166-177).

A TGF-beta inhibitor according to the present invention may be a protein, peptide, small-molecule, small-interfering RNA, antisense oligonucleotide, aptamer or antibody. The inhibitor may be naturally occurring or synthetic.

In some embodiments the TGF-beta inhibitor is a small molecule inhibitor, such as A83-01. A83-01 is a commercially available selective inhibitor of ALK4, ALK5 and ALK7 (Tocris cat. no. 2939). It is described in the catalog as a potent inhibitor of TGF-β type I receptor ALK5 kinase, type I activin/nodal receptor ALK4 and type I nodal receptor ALK7 (IC₅₀ values are 12, 45 and 7.5 nM respectively), which blocks phosphorylation of Smad2, and which only weakly inhibits ALK-1, -2, -3, -6 and MAPK activity. Other commercially available inhibitors with similar properties include, but are not limited to A77-01, LY2157299, LY2109761, LY3200882, GW788388, Pirfenidone, RepSox, SB431542, SB505124, SB525334, LY364947, SD-208 and Vactosertib. The IC50 values for these inhibitors are known in the art and the skilled person would be able to select a suitable inhibitor at suitable concentration based on the teaching provided in the examples of this application.

Therefore, in some embodiments, the TGF-beta inhibitor is an inhibitor of ALK4, ALK5 and ALK7, optionally a selective inhibitor of ALK4, ALK5 or ALK7. For example, the TGF-beta inhibitor may bind to and directly inhibit ALK4, ALK5 and/or ALK7. In some embodiments, the TGF-beta inhibitor is an inhibitor that blocks phosphorylation of Smad2. In some embodiments, the TGF-beta inhibitor is selected from A83-01, A77-01 (Tocris cat. no. 6712) LY2157299 (Selleckchem cat. no. S2230), LY2109761 (Selleckchem cat. no. S2704), LY3200882 (Selleckchem cat. no. S8772), GW788388 (Tocris cat. no. 3264), Pirfenidone, RepSox (Tocris cat. no. 3742), SB431542 (Tocris cat. no. 1614), SB505124 (Tocris cat. no. 3263), SB525334 (Tocris cat. no. 3211), LY364947 (Tocris cat. no. 2718), SD-208 (Tocris cat. no. 3269), and Vactosertib (Selleckchem cat. no. S7530). In some embodiments, the TGF-beta inhibitor is A83-01.

In some embodiments, no more than one TGF beta inhibitor is present in the expansion medium. In other embodiments, more than one TGF beta inhibitor is present in the expansion medium, e.g. 2, 3, 4 or more.

In some embodiments, the final concentration of the TGF beta inhibitor is between 1 nM and 100 μM, between 10 nM and 100 μM, between 100 nM and 10 μM, or about 1 μM, for example, wherein the final concentration of the one or more inhibitor is between 10 nM and 100 μM, between 100 nM and 10 μM, or about 500 nM. In some embodiments, the final concentration of the TGF beta inhibitor is at least 5 nM, for example, at least 50 nM, at least 100 nM, at least 300 nM, at least 450 nM, at least 475 nM, for example 5 nM-500 mM, 10 nM-100 mM, 50 nM-700 μM, 50 nM-10 μM, 100 nM-1000 nM, 350-650 nM or more preferably about 500 nM. In some embodiments, the TGF-beta inhibitor is A83-01 at a final concentration of about 500 nM.

The skilled person would appreciate that the appropriate final concentration of a TGF-beta inhibitor is dependent on the TGF-beta inhibitor in question and the skilled person would know how to determine the concentration of other TGF beta inhibitors for use in the invention.

### Prostaglandin Pathway Activator

The activator of the prostaglandin signalling pathway (also called a prostaglandin pathway activator) may be any one or more of the compounds selected from the list comprising: phospholipids, arachidonic acid (AA), prostaglandin E2 (PGE2), prostaglandin G2 (PGG2), prostaglandin F2 (PGF2), prostaglandin H2 (PGH2), prostaglandin D2 (PGD2). In some embodiments, the activator of the prostaglandin signalling pathway is PGE2 and/or AA. In some embodiments, the activator of the prostaglandin signalling pathway is PGE2.

In some embodiments, the final concentration of the activator of the of the prostaglandin signalling pathway (e.g. PGE2) is between 10 nM and 500 μM, between 10 nM, and 400 μM, between 10 nM and 300 μM, between 10 nM and 200 μM, between 10 nM and 100 μM or between 20 nM and 50 μM, or wherein the final concentration is about 1 μM.

The skilled person would appreciate that the appropriate final concentration of the activator of the of the prostaglandin signalling pathway is dependent on the activator of the of the prostaglandin signalling pathway in question and the skilled person would know how to determine the concentration of other activators of the of the prostaglandin signalling pathway for use in the invention.

cAMP Activator

The cAMP pathway activator may be any suitable activator which increases the levels of cAMP in a cell. The cAMP pathway involves activation of many types of hormone and neurotransmitter G-protein coupled receptors. Binding of the hormone or neurotransmitter to its membrane-bound receptor induces a conformational change in the receptor that leads to activation of the α-subunit of the G-protein. The activated G subunit stimulates, while the non-activated G subunit inhibits adenylyl cyclase. Stimulation of adenylyl cyclase catalyses the conversion of cytoplasmic ATP to cAMP thus increasing the levels of cAMP in the cell.

Therefore, in some embodiments, the cAMP pathway activator is an adenylyl cyclase activator or a cAMP analog. Examples of suitable adenylyl cyclase activators include forskolin, a forskolin analog and cholera toxin. Examples of forskolin analogs are known in the art and include NKH477 (e.g. catalogue no. Tocris 1603). Examples of cAMP analogs are also known in the art, and include for example, 8-bromo-cAMP. 8-bromo-cAMP is a cell-permeable cAMP analog having greater resistance to hydrolysis by phosphodiesterases than cAMP. In some embodiments, the cAMP pathway activator is therefore selected from forskolin, cholera toxin, NKH477 and 8-bromo-cAMP. In some embodiments, the cAMP pathway activator is forskolin. In some embodiments, the cAMP pathway activator is not cholera toxin.

cAMP pathway activators can be identified using methods known in the art, for example, using a competitive immunoassay which measures cAMP levels. The Catch Point® Cyclic-AMP Fluorescent Assay Kit (Molecular Devices LLC) is an example of a commercially available kit for carrying out such an immunoassay. The cAMP in the sample or standard competes with horseradish peroxidase (HRP)-labeled cAMP conjugate for binding sites on the anti-cAMP antibodies. In the absence of cAMP, most of the HRP-cAMP conjugate is bound to the antibody. Increasing concentrations of cAMP competitively decrease the amount of bound conjugate, thus decreasing measured HRP activity. A cAMP pathway activator would result in increased levels of cAMP and decreased measured HRP activity, compared to a control.

In some embodiments, the final concentration of the cAMP pathway activator (e.g. forskolin) is between 10 nM and 500 μM, between 10 nM, and 400 μM, between 10 nM and 300 μM, between 10 nM and 200 μM, between 10 nM and 100 μM or between 20 nM and 50 μM, or wherein the final concentration is about 1 μM. In some embodiments the cAMP pathway activator is forskolin. In some embodiments the final concentration of forskolin is about 1 μM.

The concentration selected may depend upon the cAMP pathway activator used and can be determined by the person skilled in the art depending upon the potency of the cAMP pathway activator. For example, NKH477 is generally more potent than 8-bromo-cAMP and forskolin. A more potent cAMP pathway activator can be used at lower concentrations to the same effect.

### BMP Inhibitor

BMPs bind as a dimeric ligand to a receptor complex consisting of two different receptor serine/threonine kinases, type I and type II receptors. The type II receptor phosphorylates the type I receptor, resulting in the activation of this receptor kinase. The type I receptor subsequently phosphorylates specific receptor substrates (SMAD), resulting in a signal transduction pathway leading to transcriptional activity.

A BMP inhibitor is defined as an agent that binds to a BMP molecule to form a complex wherein the BMP activity is neutralized, for example by preventing or inhibiting the binding of the BMP molecule to a BMP receptor. Alternatively, said inhibitor is an agent that acts as an antagonist or reverse agonist. This type of inhibitor binds with a BMP receptor and prevents binding of a BMP to said receptor. An example of a latter agent is an antibody that binds a BMP receptor and prevents binding of BMP to the antibody-bound receptor.

A BMP inhibitor may be added to the media in an amount effective to inhibit a BMP-dependent activity in a cell to at most 90%, more preferred at most 80%, more preferred at most 70%, more preferred at most 50%, more preferred at most 30%, more preferred at most 10%, more preferred 0%, relative to a level of a BMP activity in the absence of said inhibitor, as assessed in the same cell type. As is known to a skilled person, a BMP activity can be determined by measuring the transcriptional activity of BMP, for example as exemplified in Zilberberg et al., 2007. BMC Cell Biol. 8:41.

Several classes of natural BMP-binding proteins are known, including noggin (Peprotech), Chordin and chordin-like proteins (R&D systems) comprising chordin domains, Follistatin and follistatin-related proteins (R&D systems) comprising a follistatin domain, DAN and DAN-like proteins (R&D systems) comprising a DAN cysteine-knot domain, sclerostin/SOST (R&D systems), decorin (R&D systems), and alpha-2 macroglobulin (R&D systems).

Therefore, in some embodiments, the BMP inhibitor is selected from noggin, DAN, and DAN-like proteins including Cerberus and Gremlin (R&D systems). These diffusible proteins are able to bind a BMP ligand with varying degrees of affinity and inhibit their access to signalling receptors. The addition of any of these BMP inhibitors to the basal culture medium prevents the loss of stem cells. A preferred BMP inhibitor is noggin.

In some embodiments, the final concentration of the BMP inhibitor (e.g. noggin) is about 10 ng/ml to about 500 ng/ml, about 10 ng/ml to about 400 ng/ml, about 10 ng/ml to about 300 ng/ml, about 10 ng/ml to about 250 ng/ml, about 50 ng/ml to about 250 ng/ml, about 50 ng/ml to about 150 ng/ml, or wherein the final concentration is about 100 ng/ml.

### Nicotinamide

In some embodiments, the expansion medium comprises nicotinamide. Nicotinamide is an amide derivative of vitamin B3, a poly (ADP-ribose) polymerase (PARP) inhibitor, and represents the primary precursor of NAD+. It is available commercially (e.g. from Stemcell Technologies Cat. 07154). In some embodiments, Nicotinamide is present at 7-15 mM, for example about 10 mM.

### Additional Components

The expansion medium may be supplemented with one or more of the compounds selected from the group consisting of gastrin, B27, N-acetylcystein and N2. Thus in some embodiments the expansion medium described above further comprises one or more components selected from the group consisting of: gastrin, B27, N2 and N-Acetylcysteine. B27 (Invitrogen), N-Acetylcysteine (Sigma) and N2 (Invitrogen), Gastrin (Sigma) are believed to control proliferation of the cells and assist with DNA stability. In some embodiments, the expansion medium further comprises B27 and N-acetylcystein.

In some embodiments, the B27 supplement is ‘B27 Supplement minus Vitamin A’ (available from Invitrogen, Carlsbad, Calif.; www.invitrogen.com; currently catalog no. 12587010; and from PAA Laboratories GmbH, Pasching, Austria; www.paa.com; catalog no. F01-002; Brewer et al., J Neurosci Res., 35(5):567-76, 1993) may be used to formulate a culture medium that comprises biotin, cholesterol, linoleic acid, linolenic acid, progesterone, putrescine, retinyl acetate, sodium selenite, tri-iodothyronine (T3), DL-alpha tocopherol (vitamin E), albumin, insulin and transferrin. The B27 Supplement supplied by PAA Laboratories GmbH comes as a liquid 50× concentrate, containing amongst other ingredients biotin, cholesterol, linoleic acid, linolenic acid, progesterone, putrescine, retinol, retinyl acetate, sodium selenite, tri-iodothyronine (T3), DL-alpha tocopherol (vitamin E), albumin, insulin and transferrin. Of these ingredients at least linolenic acid, retinol, retinyl acetate and tri-iodothyronine (T3) are nuclear hormone receptor agonists. B27 Supplement may be added to a culture medium as a concentrate or diluted before addition to a culture medium. It may be used at a 1× final concentration or at other final concentrations. Use of B27 Supplement is a convenient way to incorporate biotin, cholesterol, linoleic acid, linolenic acid, progesterone, putrescine, retinol, retinyl acetate, sodium selenite, tri-iodothyronine (T3), DL-alpha tocopherol (vitamin E), albumin, insulin and transferrin into a culture medium of the invention. It is also envisaged that some or all of these components may be added separately to the expansion medium instead of using the B27 Supplement. Thus, the expansion medium may comprise some or all of these components. In some embodiments, retinoic acid is absent from the B27 Supplement used in the expansion medium, and/or is absent from the expansion medium.

‘N2 Supplement’ is available from Invitrogen, Carlsbad, Calif.; www.invitrogen.com; catalog no. 17502-048; and from PAA Laboratories GmbH, Pasching, Austria; www.paa.com; catalog no. F005-004; Bottenstein & Sato, PNAS, 76(1):514-517, 1979. The N2 Supplement supplied by PAA Laboratories GmbH comes as a 100× liquid concentrate, containing 500 μg/ml human transferrin, 500 μg/ml bovine insulin, 0.63 μg/ml progesterone, 1611 μg/ml putrescine, and 0.52 μg/ml sodium selenite. N2 Supplement may be added to a culture medium as a concentrate or diluted before addition to a culture medium. It may be used at a 1× final concentration or at other final concentrations. Use of N2 Supplement is a convenient way to incorporate transferrin, insulin, progesterone, putrescine and sodium selenite into a culture medium of the invention. It is of course also envisaged that some or all of these components may be added separately to the expansion medium instead of using the N2 Supplement. Thus, the expansion medium may comprise some or all of these components. In some embodiments in which the medium comprises B27, it does not also comprise N2. In some embodiments, the final concentration of N-acetylcysteine is about 1 nM to about 100 nM, about 5 nM to about 50 nM, about 10 nM to about 50 nM, about 10 nM to about 30 nM, or about 25 nM.

In some embodiments the expansion medium further comprises a ROCK inhibitor (Rho-Kinase inhibitor). A ROCK inhibitor is particularly useful for attachment of cells when establishing new cultures and/or when splitting (“passaging”) cells. Suitable ROCK inhibitors are known in the art and available commercially (including but not limited to GSK 269962, GSK 429286, H 1152 dihydrochloride, Glycyl-H 1152 dihydrochloride, SR 3677 dihydrochloride, SB 772077B dihydrochloride and Y-27632 dihydrochloride, all available from Tocris). In some embodiments, the ROCK inhibitor is at a final concentration of between 1 μM and 100 μM, between 1 μM and 50 μM, or between 5 μM and 20 μM. In some embodiments the ROCK inhibitor is Y-27632, optionally at a final concentration of about 10 μM.

### Extracellular Matrix

The methods comprise culturing cells in contact with an extracellular matrix (ECM). The ECM is an exogenous ECM (meaning that it is in addition to any extracellular matrix proteins that are naturally secreted by the epithelial stem cell or population of epithelial stem cells when in contact with the expansion medium of the invention). Any suitable ECM may be used. Cells are preferably cultured in a microenvironment that mimics at least in part a cellular niche in which said cells naturally reside. A cellular niche is in part determined by the cells and by an ECM that is secreted by the cells in said niche. A cellular niche may be mimicked by culturing said cells in the presence of biomaterials or synthetic materials that provide interaction with cellular membrane proteins, such as integrins. An ECM as described herein is thus any biomaterial or synthetic material or combination thereof that mimics the in vivo cellular niche, e.g. by interacting with cellular membrane proteins, such as integrins.

In a preferred method of the invention, cells are cultured in contact with an ECM. “In contact” means a physical or mechanical or chemical contact, which means that for separating said resulting organoid or population of epithelial cells from said extracellular matrix a force needs to be used. In some embodiments, the ECM is a three-dimensional matrix. In some embodiment, the cells are embedded in the ECM. In some embodiments, the cells are attached to an ECM. A culture medium of the invention may be diffused into a three-dimensional ECM.

In another embodiments, the ECM is in suspension, i.e. the cells are in contact with the ECM in a suspension system. In some embodiments, the ECM is in the suspension at a concentration of at least 1%, at least 2% or at least 3%. In some embodiments, the ECM is in the suspension at a concentration of from 1% to about 10% or from 1% to about 5%. The suspension method may have advantages for upscale methods.

One type of ECM is secreted by epithelial cells, endothelial cells, parietal endoderm like cells (e.g. Englebreth Holm Swarm Parietal Endoderm Like cells described in Hayashi et al. (2004) Matrix Biology 23:47 62) and connective tissue cells. This ECM comprises of a variety of polysaccharides, water, elastin, and glycoproteins, wherein the glycoproteins comprise collagen, entactin (nidogen), fibronectin, and laminin. Therefore, in some embodiments, the ECM for use in the methods of the invention comprises one or more of the components selected from the list: polysaccharides, elastin, and glycoproteins, e.g. wherein the glycoproteins comprise collagen, entactin (nidogen), fibronectin, and/or laminin. For example, in some embodiments, collagen is used as the ECM. Different types of ECM are known, comprising different compositions including different types of glycoproteins and/or different combination of glycoproteins.

The ECM can be provided by culturing ECM-producing cells, such as for example epithelial cells, endothelial cells, parietal endoderm like cells or fibroblast cells, in a receptacle, prior to the removal of these cells and the addition of isolated tissue fragments or isolated epithelial cells. Examples of extracellular matrix-producing cells are chondrocytes, producing mainly collagen and proteoglycans, fibroblast cells, producing mainly type IV collagen, laminin, interstitial procollagens, and fibronectin, and colonic myofibroblasts producing mainly collagens (type I, III, and V), chondroitin sulfate proteoglycan, hyaluronic acid, fibronectin, and tenascin-C. These are “naturally-produced ECMs”. Naturally-produced ECMs can be commercially provided. Examples of commercially available extracellular matrices include: extracellular matrix proteins (Invitrogen) and basement membrane preparations from Engelbreth-Holm-Swarm (EHS) mouse sarcoma cells (e.g. Cultrex® Basement Membrane Extract (Trevigen, Inc.) or Matrigel™ (BD Biosciences)).

Therefore, in some embodiments, is a naturally-produced ECM. In some embodiments the ECM is a laminin-containing ECM such as Matrigel™ (BD Biosciences). In some embodiments, the ECM is Matrigel™ (BD Biosciences), which comprises laminin, entactin, and collagen IV. In some embodiments, the ECM comprises laminin, entactin, collagen IV and heparin sulphate proteoglycan (e.g. Cultrex® Basement Membrane Extract Type 2 (Trevigen, Inc.)). In some embodiments, the ECM comprises at least one glycoprotein, such as collagen and/or laminin. A preferred ECM for use in a method of the invention comprises collagen and laminin. A further preferred ECM comprises laminin, entactin, and collagen IV. Mixtures of naturally-produced or synthetic ECM materials may be used, if desired.

In another embodiment, the ECM may be a synthetic ECM. For instance, a synthetic ECM, such as ProNectin (Sigma Z378666) may be used. In a further example, the ECM may be a plastic, e.g. a polyester, or a hydrogel. In some embodiments, a synthetic matrix may be coated with biomaterials, e.g. one or more glycoprotein, such as collagen or laminin.

A three-dimensional ECM supports culturing of three-dimensional epithelial organoids. The extracellular matrix material will normally be a drop on the bottom of the dish in which cells are suspended. Typically, when the matrix solidifies at 37° C., the medium is added and diffuses into the ECM. The cells in the medium stick to the ECM by interaction with its surface structure, for example interaction with integrins.

The culture medium and/or cells may be placed on, embedded in or mixed with the ECM.

For instance, in some embodiments, the single cell, population of cells, or tissue fragment is embedded in Matrigel™, which is optionally growth factor reduced and/or phenol red-free.

In some embodiments, the culture medium is placed on top of the ECM. The culture medium can then be removed and replenished as and when required. In some embodiments, the culture medium is replenished every 1, 2, 3, 4, 5, 6 or 7 days. If components are “added” or “removed” from the media, then this can in some embodiments mean that the media itself is removed from the ECM and then a new media containing the “added” component or with the “removed” component excluded is placed on the ECM.

In some embodiments, the cells are suspended in ECM in expansion medium. In some embodiments, the cells are suspended in BME (optionally ice-cold BME, optionally at a concentration of 70%) in expansion medium.

### Culturing the Epithelial Stem Cells

As the epithelial stem cells expand in the expansion medium and generate organoids, they are typically passaged (i.e. split), at regular intervals in accordance with methods known in the art. Passaging typically involves mechanically dissociating the organoids to remove them from the ECM, collecting, washing, recontacting with fresh ECM and plating at suitable ratios (e.g. 1:5 to 1:20) to allow efficient outgrowth or new organoids. The expansion medium is also typically replenished at regular intervals, as required.

Therefore, in some embodiments the method further comprises passaging the cells twice per week, once per week, once every 10 days, once every two weeks, once every 5-20 days, preferably once every 7-14 days.

In some embodiments, the method further comprises plating cells at ratios between 1:5 and 1:20.

In some embodiments, the method further comprises replenishing the expansion medium every 1-3 days, every 1-2 days, every second day, or every day. In a preferred embodiment, the method further comprises replenishing the expansion medium every 1-3 days, every 1-2 days.

In some embodiments the method comprises culturing the epithelial stem cells for at least 5 passages, for example, at least 6, at least 7, at least 8, at least 9, at least 10, at least 11, at least 12, at least 13, at least 14, at least 15, at least 16, at least 17, at least 18, at least 19, at least 20, at least 25, at least 30, at least 40, at least 50, at least 60 passages or for between 6-40 passages, for example about 8-35 passages, 10-30 passages, or 12-25 passages. In some embodiments the method comprises culturing the epithelial stem cells for 8-50, 10-50, 15-50, 20-50, or 20-40 passages. In some embodiments, the method comprises culturing the epithelial stem cells for at least 2 weeks, at least 1 month, at least 2 months, more preferably at least 3, at least 4, at least 5, at least 6, at least 7, at least 8, at least 9, at least 10, at least 15, at least 20, at least 24, at least 25, at least 30 or more months, for example 3 or more years.

Epithelial Stem Cells and their Preparation for Culture

The epithelial stem cell or population of epithelial stem cells are mammalian epithelial stem cells, optionally human or mouse epithelial stem cells. In some embodiments, the epithelial stem cells are human epithelial stem cells. In some embodiments the epithelial stem cells are characterised by Lgr5 expression.

In some embodiments, the epithelial stem cells are obtained from adult tissue, i.e. the epithelial stem cells are adult epithelial stem cells. In this context “adult” means mature tissue, i.e. includes newly-born baby or child but excludes embryonic or foetal. In some embodiments the epithelial stem cells are not derived from embryonic stem cells or embryonic stem cell lines, e.g. which have been differentiated in vitro.

Cells taken directly from tissue, i.e. freshly isolated cells, are also referred to as primary cells. In some embodiments the epithelial stem cells are primary epithelial stem cells.

Primary cell cultures can be passaged to form secondary cell cultures. With the exception cancer cells, traditional secondary cell cultures have limited lifespan. After a certain number of population doublings (e.g. 50-100 generations) cells undergo the process of senescence and stop dividing. Cells from secondary cultures can become immortalized to become continuous cell lines. Immortalization can occur spontaneously, or may be virally- or chemically-induced. Immortalized cell lines are also known as transformed cells. By contrast, the methods of the present invention allow continuous passaging of epithelial stem cells through organoid growth without immortalisation or transformation. Thus in some embodiments, the epithelial stem cells are not immortalised or transformed cells or are not derived from an immortalised cell line or a transformed cell line. An advantage of the present invention is that the epithelial stem cells, undergoing multiple rounds of expansion and passaging, retain the characteristics of primary cells and have minimal or no genotypic or phenotypic changes. The starting population of epithelial stem cell(s) in the method of the invention may therefore be obtained or derived from an existing organoid and may be further cultured and expanded to generate new cells and organoids. Thus in some embodiments, the epithelial stem cell of population of epithelial stem cells is part of an organoid or isolated from an organoid, or wherein the population of epithelial stem cells is an organoid, part of an organoid or isolated from an organoid.

The epithelial stem cells are derived from head or neck tissue. Epithelial stem cells are derived from head or neck tissue if they are primary cells from head or neck tissue, the resultant epithelial stem cells from secondary or any subsequent cultures of head and neck tissue primary cell cultures, or the resultant epithelial stem cells following one or more processing steps wherein head or neck tissue is processed. Such processing steps may include, but are not limited to the establishment of epithelial stem cell lines, extraction or isolation from a sample of head or neck tissue.

Examples of sources of epithelial stem cells from head and neck tissue include, but are not limited to, the oral cavity (e.g. floor of mouth, tongue and gingiva/alveolar process), salivary gland, larynx and pharynx.

In some embodiments the epithelial stem cells are normal cells. In alternative embodiments, the epithelial stem cells are cancer stem cells. Accordingly, the cells may be obtained from a tumour, if required. Examples of head and neck cancer include nonsquamous cell carcinoma and squamous cell carcinoma, adenocarcinoma and adenoma. In some embodiments, the tumour is head and neck nonsquamous cell carcinoma. In some embodiments, the tumour is head and neck squamous cell carcinoma. In some embodiments, tumour is adenocarcinoma, optionally salivary gland adenocarcinoma.

In some embodiments, the method comprises culturing a fragment of tissue which comprises epithelium. In some embodiments, the epithelial stem cells are isolated from a tissue fragment. For example, in the context of the head or neck, the tissue fragment may comprise surgical resections or biopsies from the oral cavity, such as from the floor of the mouth, tongue, gingiva or alveolar.

An organoid is preferably obtained using an epithelial cell from an adult tissue, optionally an epithelial stem cell from an adult tissue expressing Lgr5.

In another embodiment, an organoid originates from a single cell, optionally expressing Lgr5. Advantageously, this allows a homogenous population of cells to form. A single cell suspension comprising the epithelial stem cells can be mechanically generated, e.g. from an isolated fragment of the oral cavity. In some embodiments, the single cell suspension comprising the epithelial stem cells is generated using mechanical processes and/or enzymatic digestion. Mechanical processes include, but are not limited to dissection, micro-dissection and filtering.

In some embodiments, the starting culture is a clump or population of cells, for example, a population of cells contained in a head or neck tissue fragment. Thus, the methods of the invention are not restricted to using single cells as the starting point.

The epithelial stem cells may be obtained by any suitable isolation method known in the art. In some embodiments, the epithelial layer is micro-dissected from a tissue sample, such as a surgical specimen or biopsy, digested by an enzyme, filtered and the resultant cell suspension is plated. In some embodiments, the micro-dissection is to remove other tissue types such as fat and muscle. In some embodiments, the enzyme is trypsin, collagenase or accutase. In some embodiments, the enzyme is trypsin, optionally at 0.125% trypsin. In some embodiments, the sample is incubated at 37° C., optionally the sample is then disrupted, for example with a pipette, at repeated time intervals, such as every 2, 5, 10, or 15 minutes. In some embodiments, the sample is incubated in 0.125% trypsin at 37° C. and the sample is sheared using a pipette about every 10 minutes. In some embodiments, enzymatic digestion is performed for about 10 minutes, about 20 minutes, about 30 minutes, about 40 minutes, about 50 minutes, or about 60 minutes. In some embodiments, enzymatic digestion is completed by dilution in an appropriate medium. In some embodiments, the filtration step is performed using a 100 μm filter. In some embodiments, the resultant cell suspension from the mechanical process or enzymatic digestion is contacted with ECM and expansion medium.

One method for obtaining the epithelial stem cells for culturing is based on the fact that epithelial stem cells express Lgr5 and/or Lgr6 on their surface; these proteins belong to the large G protein-coupled receptor (GPCR) superfamily (see, for example, WO 2009/022907, the contents of which are incorporated herein in their entirety). The Lgr subfamily is unique in carrying a large leucine-rich ectodomain important for ligand binding. A preferred method therefore comprises preparing a cell suspension from said epithelial tissue as described above, contacting said cell suspension with an Lgr5 and/or 6 binding compound (such as an antibody, e.g. an anti-Lgr5 monoclonal antibody, e.g. as described in WO 2009/022907), isolating the Lgr5 and/or 6 binding compound, and isolating the stem cells from said binding compound.

Following culturing, the method may further comprise obtaining and/or isolating one or more epithelial stem cells or an organoid. For example, following culture of the stem cells, it may be useful to remove one or more stem cells and/or one or more organoids cultured in the expansion medium from the culture medium for use in subsequent applications. For example, it may be useful to isolate a single cell for subsequent differentiation.

The organoids obtained and/or isolated in a method of the invention recapitulate the characteristics of the starting epithelial stem cell or population of epithelial stem cell. For example, they retain the same genotype and phenotype, and include cells with stem-like properties (e.g. cells characterised by Lgr5 expression). This means that cells taken from diseased tissue (e.g. from cancerous tissue), faithfully model the disease in question.

### Tumour Cells and Tumour Organoids (Also Referred to Herein as “Tumoroids”)

In some embodiments, the epithelial stem cell is a tumour cell. This includes malignant, pre-malignant and non-malignant tumour cells. Tumour cells often have mutations that constitutively activate or deactivate certain growth pathways and which mean that certain factors in the culture medium that may normally be required for growth, are no longer necessary. Therefore, in some embodiments, a tumour organoid obtained from tumour epithelial stem cells is expanded and/or obtained in a culture medium that is suitable for growth of the corresponding normal tissue organoid obtained from normal stem cells, optionally with certain factors excluded from the medium.

In some embodiments, when tumour epithelial cells are culture in a method of the invention, one or more of the following are excluded from the expansion medium: Forskolin, EGF, noggin, R-spondin, TGF-beta inhibitor, nicotinamide, FGF2 and FGF10.

In some embodiments, the culture medium for culturing tumour cells further comprises an Mdm2 agonist. In some embodiments, the Mdm2 agonist is Nutlin-3.

### Organoids and Tumour Organoids

The invention provides an organoid obtainable or obtained by a method of the invention. Thus, in some embodiments, the method further comprises obtaining and/or isolating an organoid. As explained above, in some embodiments, the organoid is a tumour organoid.

An organoid obtained using the expansion methods of the invention may be referred to as an “expansion organoid”. An expansion organoid comprises at least one epithelial stem cell, which can divide and produce further epithelial stem cells or can generate differentiated progeny. It is to be understood that in a preferred expansion organoid, the majority of cells are expanding cells (i.e. dividing cells) that retain an undifferentiated phenotype. Although some spontaneous differentiation may occur, the cell population is generally an expanding population. The length of time that the organoids can continue to be expand whilst maintaining a core presence of epithelial stem cells and whilst maintaining genotypic and phenotypic integrity of the cells, is an important feature of the organoids that distinguishes them from many of the organoids in the prior art. The organoids also have a distinctive structure that arises rapidly as the cells expand and self-organise in vitro. These features are described in detail below.

Image analysis may be used to assess characteristics of cells in culture such as cell morphology; cell structures; evidence for apoptosis or cell lysis; and organoid composition and structure. Many types of imaging analysis are well known in the art, such as electron microscopy, including scanning electron microscopy and transmission electron microscopy, confocal microscopy, stereomicroscopy, fluorescence microscopy. Histological analysis can reveal basic architecture and cell types.

In some embodiments, the organoid has a three dimensional structure, i.e. the organoid is a three-dimensional organoid. In some embodiments, the organoid comprises epithelial cells. In some embodiments, the organoid comprises only epithelial cells, i.e. non-epithelial cells are absent from the organoid. This is because the culture medium of the invention is specifically designed to expand epithelial stem cells. Therefore, even if other cell types are transiently present in the culture medium, e.g. in the tissue fragment that is the starting material of the invention, these cells are unlikely to survive and instead will be replaced by the longer term expansion of the stem cells which generate a pure population of epithelial cells.

In some embodiments, the epithelial cells in the organoid surround a lumen. In some embodiments, the organoid does not comprise a lumen (in particular the tumour organoids generally do not have a lumen). In some embodiments, the epithelial cells are polarized, (meaning that proteins are differentially expressed on the apical or basolateral side of the epithelial cell). In some embodiments the lumen is a sealed lumen (meaning that a continuous cellular barrier separates the contents of the lumen from the medium surrounding the organoid). In some embodiments the organoids comprise stem cells which are able to actively divide and which are preferably able to differentiate to all major differentiated cell lineages present in the corresponding in vivo tissue, e.g. when the organoid or cell is transferred to a differentiation medium. In some embodiments, the organoid comprises basal cells on the outside and more differentiated keratinocytes (or even deposited keratin) in the center.

In some embodiments, the organoids comprise stratified epithelium. This is particularly important for HPV infection (see Example 2 and the section of the disclosure relating to uses of the organoids). By “stratified” it is meant that there are multiple (more than one) layers of cells. Such cells often tend to have their nuclei more central to the cells, i.e. not polarized. The cells in the multilayer section may organise themselves to include a gap, or lumen between the cells.

In some embodiments the organoids of the invention comprise single monolayers that are folded (or invaginated) to form two or more layers. It can sometimes be difficult to distinguish between folded (or invaginated) monolayers and regions of stratified cells. In some embodiments an organoid comprises both regions of stratified cells and regions of folded monolayers. In some embodiments the organoids of the invention have a section which is formed of multiple layers and a section comprising a single monolayer of cells. In some embodiments the organoids of the invention comprise or consist of a single monolayer of cells. In some embodiments, the organoid does not comprise a monolayer.

The organoids according to the invention may possess a layer of cells with at least one bud and a central lumen.

In some embodiments, the organoids have dense structures with a keratinised centre. Example characteristics of a keratinised centre include the presence of tonofilaments and desmosomes, i.e. in some embodiments, the keratinised centre is characterised by the presence of tonofilaments and desmosomes. In some embodiments, the organoids comprise tightly connected cells. In some embodiments, the peripheral cells are Ki67 and p63 positive and central cells are KRT13 positive. In some embodiments, the morphology of the cells of an organoid of the invention recapitulates that of the head and neck tissue from which the cells are derived. In some embodiments, the cells of an organoid of the invention have a smooth surface, whereas some cells have a folded apical surface. In some embodiments, cells show properties characteristic for keratinocytes, including tonofilament formation and tight junctions connecting it to neighbouring cells. In some embodiments, cells located towards the outside of an organoid of the invention are bigger, more rounded and/or have intact nuclei. Whereas cells towards the inside of an organoid of the invention appear to be flatter and/or lose their nucleus. The presence of differentiated keratinocytes is important for virion production (as discussed later in the examples and in the section of this disclosure relating to uses of the organoids).

In some embodiments the organoids of the invention comprise or consist of epithelial cells. In some embodiments, the organoids comprise or consist of a single layer of epithelial cells. In some embodiments non-epithelial cells are absent from the organoids. In some embodiments, the organoids of the invention comprise all the differentiated cell types that exist in head and neck tissue.

In some embodiments, the organoid has been cultured or is capable of culture in expansion media of the invention for at least 2 months, for example at least 10 weeks, at least 12 weeks, at least 14 weeks, at least 16 weeks, at least 4 months, at least 5 months, at least 6 months, at least 9 months, at least one year.

In some embodiments, the organoid has been cultured or is capable of culture for at least 5 passages, at least 10 passages, at least 15 passages, or at least 20 passages. In some embodiments, the organoid or population of epithelial stem cells are cultured for at least 10 passages.

In some embodiments, the cell number of the organoid increases exponentially over at 5 passages, 10 passages, 15 passages, or 20 passages. In a preferred embodiment, the cell number of the organoid or population of epithelial stem cells increases exponentially over 5 passages.

In some embodiments, an organoid is at least 50 μm, at least 60 μm, at least 70 μm, at least 80 μm, at least 90 μm, at least 100 μm, at least 125 μm, at least 150 μm, at least 175 μm, at least 200 μm, at least 250 μm or more in diameter at the widest point.

Within the context of the invention, a tissue fragment is a part of an adult tissue, preferably a human adult tissue. An organoid, by contrast, develops structural features through in vitro expansion, and is therefore distinguished from a tissue fragment.

In a preferred embodiment, an organoid could be cultured during at least 2, 3, 4, 5, 6, 7, 8, 9, 10 weeks or 1, 2, 3, 4, 5, 6, 7, 8, 9, 10 months or longer. In some embodiments, the organoid is expanded or maintained in culture for at least 3 months, preferably at least 4 months, at least 5 months, at least 6 months, at least 7 months, at least 9 months, or at least 12 months or more. Advantageously, use of the culture methods provided by the present invention results in organoids and/or cell populations being formed in which the number of chromosomes remains stable when the cells or organoids are cultured long-term. Thus, in some embodiments, the organoids or population of epithelial stem cells of the invention has a stable chromosome number after 2, 4, 6, 8, 10, 12 or 14 weeks or after 4, 5, 6 or more months in culture in an expansion medium of the invention. Preferably, at least 65%, at least 70%, more preferably at least 75%, more preferably at least 80%, more preferably at least 90%, more preferably at least 95%, more preferably at least 99% of the cells have the correct number of chromosomes after 2, 4, 6, 8, 10, 12 or 14 weeks or after 4, 5, 6 or more months culture in an expansion medium of the invention. For human epithelial cells, the correct number of chromosomes is 46. In some embodiments, the organoid has a normal karyotype. One method of determining the karyotype is by metaphase spread analysis. Of course, it is to be understood that tumour cells in tumour organoids, e.g. derived from tumour stem cells, would not necessarily have the correct number of chromosomes as genomic instability is a feature of certain cancers.

In some embodiments, an organoid of the invention is a tumour organoid. A tumour organoid of the invention may be obtained by culturing an epithelial tumour cell in the expansion medium of the invention. In a preferred embodiment the tumour is head and neck squamous cell carcinoma.

In some embodiments, the epithelial stem cell or population of epithelial stem cells comprises tumour cells. In a preferred embodiment the tumour is head and neck squamous cell carcinoma.

In some embodiments, tumour organoids are dense structures, similar to the resultant organoids from the culture of non-cancerous epithelial stem cells. In other embodiments, tumour organoids are cystic structures. In some embodiments, tumour organoids derived from different patients show different morphologies. Organoid morphology may be assessed using techniques such as image analysis, including brightfield microscopy.

In some embodiments, tumour organoids of the invention retain tumour-specific histopathological changes or characteristics. Such changes can be identified by comparing organoids derived from the tumour tissue and the adjacent epithelium from the same patient and/or by comparing the tumour organoid with the primary tissue specimen.

In some embodiments, the tumour organoid is a dense or cystic structure. In some embodiments, the tumour organoid comprises transformed epithelial tumour cells. In some embodiments, the tumour organoids predominantly comprise transformed epithelial tumour cells. In some embodiments, the tumour organoids contain only transformed epithelial tumour cells (i.e. non-transformed epithelial cells are absent from the tumour organoid). In some embodiments, the tumour organoid does not comprise immune-, connective tissue- and/or vessel-elements. The skilled person would appreciate that the presence of transformed epithelial tumour cells could be assessed my various methods, including keratin (KRT5) immuno-staining.

In some embodiments, tumour organoids of the invention are selected by culture in a culture medium of the invention that further comprises an Mdm2 agonist, such as Nutlin-3. The Mdm2 agonist may be present for the whole of or part of the culture process. In some embodiments, Nutlin-3 is present at a concentration of about 10 μM. Nutlin-3 prevents the growth of p53 wild type cells. P53 is a tumour suppressor gene. Thus decreased p53 function, for example caused by a mutation in the p53 gene or other mis-regulation of p53, is a common cause of tumorigenesis. Culturing organoids in the presence of Nutlin-3 therefore selected organoids with decreased levels of p53 or deceased p53 activity and can be used as a method for selecting tumour organoids.

In some embodiments, organoids and tumour organoids of the invention have different transcriptome profiles. In some embodiments, organoids of the invention cluster together and tumour organoids of the invention cluster together based on a principle component analysis of a transcriptome analysis. In some embodiments, organoids and tumour organoids of the invention have differential gene expression. For example, KLK6 and/or EHF are downregulated in HNSCC tumour organoids. Whereas, SLCOB1, HOXC13, CALB1, NTS and/or BCHE are upregulated in HNSCC tumour organoids. Further genes which may show differential expression between non-tumour organoids and tumour organoids include SDC2, HOXA1, NXPE3 and/or HOXC10. In some embodiments, when corresponding non-tumour organoids and tumour organoids are compared there are more than 50 differentially expressed genes, more than 100 differentially expressed genes, more than 200 differentially expressed genes or more than 300 differentially expressed genes.

In some embodiments, a tumour organoid of the invention has an enrichment of the variable allele frequency of detected mutations relative to the tumour. Variant allele frequency refers to the frequency that a mutation is detected within a sample, for example this can be given as the percentage of reads from a sequencing analysis that contain a particular mutation.

In some embodiments, the genetic alterations in a tumour organoid of the invention recapitulate the genetic alterations of the tumour from which the organoid is derived. Genetic alterations include single nucleotide variant and small insertions and deletions. In some embodiments, organoids of the invention which are not derived from cancer cells contain fewer genetic alterations than tumour organoids of the invention which are derived from cancer cells.

Chromosome mis-segregation underlies the aneuploidies frequently observed in human tumours. Chromosome segregation errors include anaphase bridges and bi-nucleated cells undergoing multipolar division. Increase mis-segregation rates result in the phenotype known as chromosomal instability, which is commonly observed in cancers. In some embodiments, tumour organoids demonstrate chromosome instability. In some embodiments, the tumour organoid has an increased rate of chromosome segregation errors compared to organoids derived from cells which are not cancer cells. In some embodiments, the tumour organoid has an increased rate of chromosome segregation errors, including anaphase bridges and/or bi-nucleated cells undergoing multi-polar division.

In some embodiments, a tumour organoid of the invention does not have a normal karyotype. In some embodiments, the tumour organoid displays aneuploidy. The karyotype of an organoid, i.e. the number of chromosomes, can be determined by metaphase spread analysis.

In some embodiments, a tumour organoid of the invention is tumourogenic when subcutaneously transplanted. The tumourogenic potential of human organoids can be determined by subcutaneous transplantation of said organoids into mice. Typically the subcutaneous transplantation of non-tumour organoids does not result in tumour formation or at least outgrowth of the organoid. In some embodiments, a tumour organoid of the invention retains its tumourigenic potential in culture and can form tumours with similar features to the parental tumour following subcutaneous transplantation. Such characteristics may include levels of atypia that are regarded cancerous, tripolar mitotic figures, nuclear polymorphism and/or muscle invasion.

The expansion organoid of the invention preferably comprises at least 50% viable cells, more preferred at least 60% viable cells, more preferred at least 70% viable cells, more preferred at least 80% viable cells, more preferred at least 90% viable cells. Viability of cells may be assessed using Hoechst staining or Propidium Iodide staining in FACS. In some embodiments, there is provided one or more frozen organoids of the invention. Also provided is a method for preparing organoids for freezing comprising dissociating organoid cultures and mixing them with a freezing medium such as Recovery cell culture freezing medium (Gibco) and freezing following standard procedures. A method for thawing frozen organoids is also provided which comprises thawing frozen organoids, embedding the thawed organoids in an extracellular matrix (e.g. Matrigel) and culturing the organoids in an expansion medium of the invention. Advantageously, initially after thawing the culture medium may be supplemented with Y-27632, for example, about 10 uM Y-27632. In some embodiments, the culture medium is supplemented with Y-27632 for the first 1, 2, 3, 4, 5 or less days after thawing, preferably for the first 3 or 4 days. In some embodiments, Y-27632 is not present in the culture medium after the first 3, 4, 5, 6 or more days, preferably after the first 3 or 4 days. This freezing method can be used for expansion organoids of the invention.

In some embodiments, the organoid or tumour organoid of the invention is cryopreserved.

### Differentiation

The expansion organoids, or cells from the expansion organoids can be transferred to a differentiation medium and be allowed to or induced to differentiate into all major differentiated cell lineages present in head and neck tissue.

In some embodiments, the method of the invention comprises a first step of culturing an epithelial stem cell or population of stem cells in an expansion medium and a second step of culturing the expanded cells or expansion organoid in a differentiation medium.

In some embodiments, the differentiation medium comprises the expansion medium from which one or more of the following are not included: one or more mitogenic growth factor, a TGF-beta inhibitor, an activator of the prostaglandin signalling pathway, one or more Wnt agonist, a cAMP pathway activator, a BMP inhibitor and nicotinamide. In some embodiments, the differentiation medium comprises a basal medium for animal or human cells.

In some embodiments, culturing the population of epithelial stem cells in the differentiation medium increases expression levels of KRT13. The skilled person is aware of different techniques to determine gene or protein expression, including quantitative PCR.

### Expansion Medium

The invention provides an expansion medium as described herein. The expansion medium is a cell culture medium suitable for expanding epithelial stem cells. An expansion medium that is used in a method of the invention comprises any suitable basal medium. Basal media for cell culture typically contain a large number of ingredients, which are necessary to support maintenance of the cultured cells. Suitable combinations of ingredients can readily be formulated by the skilled person, taking into account the following disclosure. A basal medium for use in the invention will generally comprises a nutrient solution comprising standard cell culture ingredients, such as amino acids, vitamins, lipid supplements, inorganic salts, a carbon energy source, and a buffer, as described in more detail in the literature and below. In some embodiments, the culture medium is further supplemented with one or more standard cell culture ingredient, for example selected from amino acids, vitamins, lipid supplements, inorganic salts, a carbon energy source, and a buffer.

Suitable basal media will be known to the skilled person and are available commercially, e.g. non-limiting examples include Dulbecco's Modified Eagle Media (DMEM), Advanced-DMEM, Minimal Essential Medium (MEM), Knockout-DMEM (KO-DMEM), Glasgow Minimal Essential Medium (G-MEM), Basal Medium Eagle (BME), DMEM/Ham's F12, Advanced DMEM/Ham's F12, Iscove's Modified Dulbecco's Media and Minimal Essential Media (MEM), Ham's F-10, Ham's F-12, Medium 199, and RPMI 1640 Media.

For example, the basal medium may be Advanced-DMEM, preferably supplemented with glutamax, penicillin/streptomycin and HEPES.

It is preferred that the expansion medium does not comprise an undefined component such as fetal bovine serum or fetal calf serum. Various different serum replacement formulations are commercially available and are known to the skilled person. Where a serum replacement is used, it may be used at between about 1% and about 30% by volume of the medium, according to conventional techniques. In some embodiments, the expansion medium is serum free.

The preferred culture methods of the invention are also advantageous because feeder cells are not required. Feeder cell layers are often used to support the culture of stem cells, and to inhibit their differentiation. The use of feeder cells is undesirable, because it complicates passaging of the cells (the cells must be separated from the feeder cells at each passage, and new feeder cells are required at each passage). The use of feeder cells can also lead to contamination of the desired cells with the feeder cells. This is clearly problematic for any medical applications, and even in a research context, complicates analysis of the results of any experiments performed on the cells.

Therefore, in some embodiments, the methods, media and compositions of the invention are feeder cell-free. A composition is conventionally considered to be feeder cell-free if the cells in the composition have been cultured for at least one passage in the absence of a feeder cell layer. A feeder cell-free composition of the invention will normally contain less than about 5%, less than about 4%, less than about 3%, less than about 2%, less than about 1% feeder cells (expressed as a % of the total number of cells in the composition) or preferably no feeder cells at all.

A culture medium of the invention will normally be formulated in deionized, distilled water. A culture medium of the invention will typically be sterilized prior to use to prevent contamination, e.g. by ultraviolet light, heating, irradiation or filtration. The culture medium may be frozen (e.g. at −20° C. or −80° C.) for storage or transport. The medium may contain one or more antibiotics to prevent contamination. The medium may have an endotoxin content of less than 0.1 endotoxin units per ml, or may have an endotoxin content less than 0.05 endotoxin units per ml. Methods for determining the endotoxin content of culture media are known in the art.

A preferred cell culture medium is a defined synthetic medium that is buffered at a pH of 7.4 (preferably with a pH 7.2-7.6 or at least 7.2 and not higher than 7.6) with a carbonate-based buffer, while the cells are cultured in an atmosphere comprising between 5% and 10% CO2, or at least 5% and not more than 10% CO2, preferably 5% CO2.

The invention also provides a composition or cell culture vessel comprising cells and/or organoids according to any one of the aspects of the invention described above, and a culture medium according to any one of the aspects of the invention described above. For example, such a composition or cell culture vessel may comprise any number of cells or organoids cultured according to a method of the invention, in a culture medium as described above.

According to a still further aspect of the invention, there is provided a hermetically-sealed vessel containing a culture medium of the invention. In some embodiments, the culture medium is an expansion medium. In some embodiments, the culture medium is a differentiation medium. Hermetically-sealed vessels may be preferred for transport or storage of the culture media, to prevent contamination. The vessel may be any suitable vessel, such as a flask, a plate, a bottle, a jar, a vial or a bag.

### Exemplified Expansion Medium

The invention provides a method for culturing an epithelial stem cell or a population of epithelial stem cells derived from head or neck tissue, wherein the method comprises culturing one or more epithelial stem cells in contact with an extracellular matrix in the presence of an expansion medium, the expansion medium comprising a basal medium for animal or human cells and further comprising: one or more mitogenic growth factor, a TGF-beta inhibitor, an activator of the prostaglandin signalling pathway, one or more Wnt agonist, a cAMP pathway activator, a BMP inhibitor and nicotinamide.

In some embodiments, in the expansion medium the mitogenic growth factor comprises FGF2, and the Wnt agonist comprises a GSK-3 inhibitor. The presence of both FGF2 and a GSK-3 inhibitor, for example CHIR-99021, in the expansion medium is particularly advantageous for culturing epithelial stem cells and organoids derived from head and neck tissue.

In some embodiments, in the expansion medium the mitogenic growth factor comprises FGF2, the Wnt agonist comprises a GSK-3 inhibitor, and the cAMP pathway activator is forskolin.

In some embodiments, in the expansion medium the mitogenic growth factor comprises FGF2, the Wnt agonist comprises a GSK-3 inhibitor, the cAMP pathway activator is forskolin, and the activator of the prostaglandin signalling pathway is PGE2.

In some embodiments, in the expansion medium the mitogenic growth factor comprises FGF2, the Wnt agonist comprises a GSK-3 inhibitor, and the activator of the prostaglandin signalling pathway is PGE2.

In some embodiments, in the expansion medium the mitogenic growth factor comprises FGF2, and wherein the Wnt agonist comprises R-spondin and GSK-3 inhibitor.

In some embodiments, in the expansion medium the mitogenic growth factor is EGF, FGF10 and FGF2, the TGF-beta inhibitor is A83-01, the Wnt agonist comprises R-spondin and GSK-3 inhibitor, the cAMP pathway activator is forskolin, the activator of the prostaglandin signalling pathway is PGE2, and the BMP inhibitor is noggin.

In some embodiments, in the expansion medium the final concentration of nicotinamide is about 1 mM to 100 mM, the final concentration of EGF is about 0.1 ng/ml to 500 ng/ml, the final concentration of FGF10 is about 0.1 ng/ml to 500 ng/ml, the final concentration of FGF2 is about 0.1 ng/ml to 500 ng/ml, the final concentration of A83-01 is about 1 nM to 100 μM, the final concentration of R-spondin is about 150 ng/ml to 250 ng/ml, the final concentration of CHIR-99021 is about 10 nM to 500 μM, the final concentration of forskolin is about 20 nM and 50 μM the final concentration of PGE2 is about 10 nM to 500 μM, and the final concentration of noggin is about 50 ng/ml to 150 ng/ml.

In some embodiments, in the expansion medium the final concentration of nicotinamide is about 10 mM, the final concentration of EGF is about 50 ng/ml, the final concentration of FGF10 is about 10 ng/ml, the final concentration of FGF2 is about 5 ng/ml, the final concentration of A83-01 is about 500 nM, the final concentration of R-spondin is about 200 ng/ml, the final concentration of CHIR-99021 is about 3 μM or 0.3 μM, the final concentration of forskolin is about 1 μM, the final concentration of PGE2 is about 1 μM, and the final concentration of noggin is about 100 ng/ml.

Another aspect of the invention provides a method for culturing a mouse epithelial stem cell or a population of mouse epithelial stem cells, wherein the method comprises culturing one or more epithelial stem cells in contact with an extracellular matrix in the presence of an expansion medium, the expansion medium comprising a basal medium and further comprising: one or more mitogenic growth factors, one or more Wnt agonists and nicotinamide; wherein the epithelial stem cells are derived from head or neck tissue. In some embodiments, the expansion medium comprises a basal medium, B27, N acetyl-L-cysteine, nicotinamide, R-spondin, EGF and FGF10. In some embodiments, the mitogenic growth factor is EGF and FGF10, the Wnt agonist comprises R-spondin. In some embodiments, the expansion medium further comprises B27 and/or N acetyl-L-cysteine. In some embodiments, the final concentration of nicotinamide is about 10 mM, the final concentration of EGF is about 50 ng/ml, the final concentration of FGF10 is 10 ng/ml and the final concentration of R-spondin is about 100 ng/ml.

### Compositions and Other Forms of the Invention

The invention provides a composition comprising epithelial stem cells derived from head and neck tissue and an expansion medium according to the invention.

The invention also provides a composition comprising an organoid of the invention (or cells directly obtained from an organoid of the invention) and a culture medium, optionally an expansion medium of the invention.

The invention also provides a composition comprising an expansion medium of the invention and an extracellular matrix. In some embodiments this expansion medium further comprises epithelial stem cells derived from head and neck tissue and/or an organoid of the invention.

The invention also provides a culture medium supplement that can be used to produce an expansion medium of the invention. The supplement may contain one or more of the expansion medium components as disclosed herein. In some embodiments, the culture medium supplement does not contain the basal medium. For example, in some embodiments, the culture medium supplement consists of one or more mitogenic growth factor, a TGF-beta inhibitor, an activator of the prostaglandin signalling pathway, one or more Wnt agonist, a cAMP pathway activator, a BMP inhibitor and nicotinamide.

The invention also provides a hermetically-sealed vessel containing an expansion medium or culture medium supplement of the invention. Hermetically-sealed vessels may be preferred for transport or storage of the culture media or culture media supplements disclosed herein, to prevent contamination. The vessel may be any suitable vessel, such as a flask, a plate, a bottle, a jar, a vial or a bag.

The invention also provides a kit comprising an expansion medium, a culture medium supplement, epithelial stem cells derived from head and neck tissue, organoids of the invention and/or a composition of the invention.

### Uses of Organoids

Organoids of the invention faithfully represent the in vivo situation. This is true both for organoids grown from normal tissue and for organoids grown from diseased tissue. Therefore, organoids of the invention are useful in medicine and diagnostics, and in research and drug development. As well as providing normal ex vivo cell/organ models, the organoids of the invention can be used as ex vivo models of disease and/or infection. Therefore, organoids of the invention can be used for drug screening, including drug discovery and validation, target discovery and validation, toxicology, infection models and other research purposes. Diseases that can be studied by the organoids of the invention include but are not limited to genetic diseases, metabolic diseases, pathogenic diseases and inflammatory diseases. The organoids are also suitable for transplantation, and thus have potential for regenerative medicine. In addition, organoids can be grown quickly from any individual's cells, and thus can be used for identifying suitable treatment regimes, e.g. in the context of personalised medicine. Several uses of organoids are described in earlier applications (e.g. in WO2009/022907, WO2010/090513, WO2012/014076, WO2012/168930, WO2015/173425, WO2016/083613, and WO2016/083612) and these uses also apply to the head and neck organoids of the invention.

The examples provide details on the properties of the organoids that make them suitable for these uses, and further specific examples are provided below.

### Infection Model

The inventors have shown that organoids of the invention can be used for culturing a pathogen and thus be used as ex vivo infection models (e.g. see Example 2). Therefore, the invention provides a head and neck tissue disease model that represents an infected state. In some embodiments, the invention provides the use of an organoid of the invention (or cells directly obtained from said organoid) in a model for infection. In some embodiments, the organoid or cells directly obtained from said organoid are infected with a pathogen, such as a virus, which can or is known to be able to infect keratinocytes or epithelial cells of head and neck tissue. Therefore, in some embodiments, the infection is a viral infection. Viruses that may be used for such infection models include Herpes Simplex Viruses, such as Herpes Simplex Virus type 1, and Human Papilloma Viruses, such as Human Papilloma Virus type 16. In some embodiments, the organoid is infected with Herpes Simplex Virus, optionally wherein the Herpes Simplex Virus is HSV1. In some embodiments, the organoid is infected with Human Papilloma Virus, optionally wherein the Human Papilloma Virus is HPV16.

The infection of an organoid of the invention or population of cells derived from said organoid with a virus, which is known to infect the tissue type from which the organoid is derived, may validate the use of the organoid or population of cells derived from said organoid as an ex vivo model. For example, it is important for HPV infection that HPV has access to basal cells of the stratified epithelium, while for virion production, differentiated keratinocytes are important. A model which retains this stratification in vitro, such as the one seen in Example 2, thus faithfully represents the in vivo situation and provides ex vivo cell/organ models.

Infection studies may include observing or monitoring the spread of the infection using life imaging, for example by labelling a viral component with a marker, for example a fluorescent label that may be detected using fluorescence microscopy. In some embodiments, features of a viral infection may also be monitored by quantifying the abundance of viral nucleic acid, for example viral replication can be determined by quantifying viral nucleic acid levels, such as the level of Human Papilloma Virus DNA. In some embodiments, the use of an organoid of the invention or cells from said organoid in infection studies may involve the use of an inhibitor, for example a thymidine kinase inhibitor, which decreases the infection of organoid or population of cells derived from said organoid.

In some embodiments, the invention provides a method of modelling viral infection comprising the step of culturing an epithelial stem cell or a population of epithelial stem cells derived from head or neck tissue in accordance with the invention, and further comprising infecting the resultant organoid or population of cells with a virus, optionally wherein the virus is a Herpes Simplex Virus, such as HSV1, or a Human Papilloma Virus, such as HPV16. In some embodiments, the method of modelling viral infection comprises producing virus and contacting the cultured population of epithelial stem cells with said virus. In some embodiments, producing the virus comprises collecting the supernatant into which the virus is produced. In some embodiments, the method of modelling viral infection comprises fractionating the virus supernatant, optionally wherein the viral titer of the fractions is determined. The viral titer is the concentration of the virus.

### Drug Screening

The inventors have shown that the organoids of the invention provide a platform for drug-screening (e.g. see Examples 7 and 8). Therefore, the invention provides the use of an organoid (or cells directly obtained from said organoid) in drug screening, target validation, target discovery, toxicology or a toxicology screen.

The cells are preferably exposed to multiple concentrations of a test agent for a certain period of time. At the end of the exposure period, the cultures are evaluated. The organoid can also be used to identify drugs that specifically target epithelial carcinoma cells. It will be understood by the skilled person that the organoids of the invention would be widely applicable as drug screening tools for infectious, inflammatory and neoplastic pathologies of the head and neck and other diseases of the head and neck. In some embodiments, the invention provides the use of an organoid in drug screening, target validation, target discovery, toxicology, toxicology screens or an ex vivo cell/organ model. In some embodiments, the invention provides the use of an organoid in an ex vivo method to predict a clinical outcome. In some embodiments, the organoids of the invention could be used for screening for cancer drugs.

In some embodiments, the organoids of the invention can be used to test libraries of chemicals, antibodies, natural product (plant extracts), etc for suitability for use as drugs, cosmetics and/or preventative medicines. For instance, in some embodiments, a cell biopsy from a patient of interest, such as tumour cells from a cancer patient, can be cultured using culture media and methods of the invention and then treated with a chemical compound or a chemical library. It is then possible to determine which compounds effectively modify, kill and/or treat the patient's cells. This allows specific patient responsiveness to a particular drug to be tested thus allowing treatment to be tailored to a specific patient. Thus, this allows a personalised medicine approach. In some embodiments, the drug screening method is an ex vivo method to guide personalised theory and/or predict a clinical outcome. In some embodiments, the invention provides an organoid for use in a method to guide personalised therapy. The added advantage of using the organoids for identifying drugs in this way is that it is possible to screen normal organoids (organoids derived from healthy tissue) to check which drugs and compounds have minimal effect on healthy tissue. This allows screening for drugs with minimal off target activity or unwanted side effects.

In some embodiments, the invention provides a method for testing the effect of a candidate compound, wherein the method comprises:
culturing epithelial stem cell or a population of epithelial stem cells derived from head or neck tissue according to the method of the invention, optionally for less than 21 days;
exposing the resultant population of cells or the resultant organoid to one or a library of candidate compounds;
evaluating said expanded organoids for any effects,
identifying the candidate molecule that causes said effects as a potential drug; and optionally
providing said candidate molecule, e.g. as a drug.

In some embodiments, the method for testing the effect of a candidate compound comprises exposing the organoid to radiation in the presence or absence of a candidate compound. In some embodiments, an evaluated effect in the method for testing the effect of a candidate compound is selected from the list comprising: a reduction in, or loss of, proliferation, a morphological change, cell death or a change in gene or protein expression.

A library of candidate molecules comprises more than one candidate molecule. Candidate compounds may include, but are not limited to: Carboplatin, Cetuximab, Cisplatin, Alpelisib, Vemurefenib, Niraparib, Everolimus and AZD4547.

In some embodiments, the invention provides a method comprising:
culturing epithelial stem cell or a population of epithelial stem cells derived from head or neck tissue according to the method of the invention, optionally for less than 21 days;
exposing the resultant organoid or a population of cells derived from the resultant organoid to a treatment, such as radiation, and/or to one or a library of candidate molecules;
evaluating said organoid or population of cells for any effects of a candidate molecule; and
correlating said effect with a feature of the organoid, for example the presence of one or more genetic mutations, such as mutations in the EGFR signalling pathway, including PIK3CA, KRAS, HRAS or BRAF.

In some embodiments, the invention provides a method comprising:
culturing epithelial stem cell or a population of epithelial stem cells derived from head or neck tissue according to the method of the invention, optionally for less than 21 days;
exposing the resultant organoid or a population of cells derived from the resultant organoid to a treatment, such as radiation, and/or to one or a library of candidate molecules;
evaluating said organoid or population of cells for any effects of a candidate molecule;
comparing said effect with standard values and/or previous observations; and optionally
predicting clinical outcome and/or selecting a personalised medicine.

In some embodiments, the organoid is derived from a patient biopsy. In some embodiments, the candidate molecule that causes a desired effect on the organoid or population of cells derived from said organoid is administered to said patient. Accordingly, in one aspect, there is provided a method of treating a patient comprising:
a) obtaining a biopsy from the diseased head or neck tissue of interest in the patient;
b) culturing the biopsy to obtain an organoid;
c) identifying a suitable drug using a screening method of the invention; and
d) treating said patient with the drug obtained in step (c).

In some embodiments, the invention provides a method for selecting a treatment regime for a patient, wherein the method comprises the steps of:
optionally obtaining a biopsy from the head or neck tissue of the patient;
culturing the biopsy, a tissue fragment of the biopsy, an epithelial stem cell of the biopsy or a population of epithelial stem cells of the biopsy according to said method to obtain an organoid or a tumour organoid;
exposing the resultant organoid to a treatment regime, including radiation and/or one or more candidate compounds;
evaluating said organoid for any effects;
identifying the treatment of regime that causes said effects; and
optionally providing said treatment to the patient.

Transplantation and Medicine

The inventors have shown that organoids recapitulate characteristics of the tissue from which they are derived when transplanted into mice (e.g. see Example 6 which shows that HNSCC organoids retain tumorigenic potential in culture and can form HNSCC with features similar to the parental tumour, upon transplantation into mice). The transplantation of non-tumour organoids did not result in out-growth of the organoid. The invention provides the use of organoids in regenerative medicine and/or transplantation. The invention also provides methods for treatment wherein the method comprises transplanting an organoid into an animal or human.

In some embodiments, the invention provides an organoid for use in diagnostics or medicine. In some embodiments, the invention provides an organoid for use in diagnostics or medicine, optionally in personalised medicine or diagnostics, or regenerative medicine. In some embodiments, the invention provides method of treating a disease comprising the step of administering an organoid of the invention. In some embodiments, the invention provides the Use of an organoid of invention in the manufacture of a medicament for treating a disease. In some embodiments, the invention provides

### Definitions

As used herein, the verb “to comprise” and its conjugations is used in its non-limiting sense to mean that items following the word are included, but items not specifically mentioned are not excluded. In addition the verb “to consist” may be replaced, if necessary, by “to consist essentially of” meaning that a product as defined herein may comprise additional component(s) than the ones specifically identified, said additional component(s) not altering the unique characteristic of the invention. In addition a method as defined herein may comprise additional step(s) than the ones specifically identified, said additional step(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”.

As used herein, the term “about” or “approximately” means that the value presented can be varied by +/−10%. The value can also be read as the exact value and so the term “about” can be omitted. For example, the term “about 100” encompasses 90-110 and also 100.

All patent and literature references cited in the present specification are hereby incorporated by reference in their entirety.

Any reference to a method for treatment comprising administering an agent to a patient, also covers that agent for use in said method for treatment, as well as the use of the agent in said method for treatment, and the use of the agent in the manufacture of a medicament.

The following examples are offered for illustrative purposes only, and are not intended to limit the scope of the present invention in any way.

### EMBODIMENTS

The invention also provides the following numbered embodiments.

1. A method for culturing an epithelial stem cell or a population of epithelial stem cells derived from head or neck tissue, wherein the method comprises culturing one or more epithelial stem cells in contact with an extracellular matrix in the presence of an expansion medium, the expansion medium comprising a basal medium for animal or human cells and further comprising: one or more mitogenic growth factor, a TGF-beta inhibitor, an activator of the prostaglandin signalling pathway, one or more Wnt agonist, a cAMP pathway activator, a BMP inhibitor and nicotinamide.

2. The method of embodiment 1, wherein in the expansion medium the one or more Wnt agonist comprises an R-spondin family protein and/or a GSK-3 inhibitor.

3. The method of embodiment 2, wherein the R-spondin family protein is selected from R-spondin-1, R-spondin-2, R-spondin-3, R-spondin-4 or a fragment, analog or variant thereof.

4. The method of embodiments 2 or 3, wherein the final concentration of R-spondin family protein is about 10 ng/ml to about 500 ng/ml, about 10 ng/ml to about 400 ng/ml, about 10 ng/ml to about 300 ng/ml, about 10 ng/ml to about 250 ng/ml, about 50 ng/ml to about 250 ng/ml, about 100 ng/ml to about 250 ng/ml, or about 150 ng/ml to about 250 ng/ml.

5. The method of embodiment 4, wherein the R-spondin family protein is R-spondin 1, R-spondin 2, R-spondin 3 or R-spondin 4 and the final concentration is about 150 ng/ml to about 250 ng/ml, or wherein the final concentration is about 200 ng/ml.

6. The method of any one of the preceding embodiments, wherein the one or more Wnt agonist further comprises a Wnt ligand, optionally selected from one or more of Wnt-3a, Wnt-5 or Wnt-6a.

7. The method of any one of embodiments 2-6, wherein the final concentration of each of the GSK-3 inhibitor is between 10 nM and 500 μM, between 10 nM, and 400 μM, between 10 nM and 300 μM, between 10 nM and 200 μM, between 10 nM and 100 μM, between 20 nM and 50 μM or between 100 nM and 50 μM, or wherein the final concentration is about 3 μM, or wherein the final concentration is about 0.3 μM.

8. The method of any one of the preceding embodiments, wherein the GSK-3 inhibitor is CHIR-99021, optionally at a final concentration of about 3 μM or at a final concentration of about 0.3 μM.

9. The method of any one of the preceding embodiments, wherein in the expansion medium the one or more mitogenic growth factor binds to a receptor tyrosine kinase such as EGFR, an FGFR or HGFR, optionally wherein the one or more mitogenic growth factors are selected from EGF, FGF and HGF.

10. The method of embodiment 9, wherein the mitogenic growth factor comprises a factor which binds to EGFR and a factor which binds to FGFR, optionally wherein the one or more mitogenic growth factor comprises EGF and FGF.

11. The method of embodiment 10, wherein the FGF comprises FGF2 and FGF10.

12. The method of any one of the preceding embodiments, wherein the final concentration of each mitogenic growth factor is between 0.1 ng/ml and 500 ng/ml, between 0.1 ng/ml and 400 ng/ml, between 0.1 ng/ml and 300 ng/ml, between 0.1 ng/ml and 200 ng/ml, between 0.1 ng/ml and 100 ng/ml, between 1 ng/ml and 100 ng/ml, or wherein the final concentration of a mitogenic growth factor is about 1 ng/ml, 2 ng/ml, 5 ng/ml, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 250 ng/ml or 500 ng/ml.

13. The method of any one of the preceding embodiments, wherein the one or more mitogenic growth factor comprises EGF, FGF10 and FGF2, optionally wherein:
the EGF is at a final concentration of about 50 ng/ml,
the FGF10 is at a final concentration of about 10 ng/ml, and/or
the FGF2 is at a final concentration of about 5 ng/ml.

14. The method of any one of the preceding embodiments, wherein in the expansion medium the TGF-beta inhibitor is an inhibitor of the ALK4, ALK5 or ALK7 signalling pathway, optionally wherein the TGF-beta inhibitor is a small molecule inhibitor, optionally A83-01.

15. The method of any one of the preceding embodiments, wherein the final concentration of the TGF-beta inhibitor is between 1 nM and 100 μM, between 10 nM and 100 μM, between 100 nM and 10 μM, or about 1 μM, for example, wherein the final concentration of the one or more inhibitor is between 10 nM and 100 μM, between 100 nM and 10 μM, or about 500 nM.

16. The method of any one of the preceding embodiments, wherein the TGF-beta inhibitor is A83-01, optionally wherein the final concentration is about 500 nM.

17. The method any one of the preceding embodiments, wherein in the expansion medium the activator of the prostaglandin signalling pathway is selected from the list comprising: a phospholipid, Arachidonic acid (AA), prostaglandin E2 (PGE2), prostaglandin G2 (PGG2), prostaglandin F2 (PGF2), prostaglandin H2 (PGH2), and prostaglandin D2 (PGD2).

18. The method of any one of the preceding embodiments, wherein the final concentration of the activator of the of the prostaglandin signalling pathway is between 10 nM and 500 μM, between 10 nM, and 400 μM, between 10 nM and 300 μM, between 10 nM and 200 μM, between 10 nM and 100 μM or between 20 nM and 50 μM, or wherein the final concentration is about 1 μM.

19. The method of any one of the preceding embodiments, wherein the activator of the prostaglandin signalling pathway is PGE2, optionally wherein the final concentration PGE2 is about 1 μM.

20. The method of any one of the preceding embodiments, wherein in the expansion medium the cAMP pathway activator is an adenylyl cyclase activator, optionally selected from the list comprising: forskolin, a forskolin analog and cholera toxin.

21. The method of any one of the preceding embodiments, wherein the final concentration of the cAMP pathway activator is between 10 nM and 500 μM, between 10 nM, and 400 μM, between 10 nM and 300 μM, between 10 nM and 200 μM, between 10 nM and 100 μM or between 20 nM and 50 μM, or wherein the final concentration is about 1 μM.

22. The method of any one of the preceding embodiments, wherein the cAMP pathway activator is forskolin, optionally wherein the final concentration is about 1 μM.

23. The method of any one of the preceding embodiments, wherein in the expansion medium the BMP inhibitor is noggin.

24. The method of any one of the preceding embodiments, wherein the final concentration of the BMP inhibitor is about 10 ng/ml to about 500 ng/ml, about 10 ng/ml to about 400 ng/ml, about 10 ng/ml to about 300 ng/ml, about 10 ng/ml to about 250 ng/ml, about 50 ng/ml to about 250 ng/ml, about 50 ng/ml to about 150 ng/ml, or wherein the final concentration is about 100 ng/ml.

25. The method of any one of the preceding embodiments, wherein the BMP inhibitor is noggin at a final concentration of about 100 ng/ml.

26. The method of any one of the preceding embodiments, wherein the final concentration of nicotinamide is between 1 μM and 500 mM, between 1 μM and 250 mM, between 100 μM and 250 mM, between 500 μM and 250 mM, between 1 mM and 250 mM, between 1 mM and 100 mM, or wherein the final concentration is about 10 mM.

27. The method of any one of the preceding embodiments, wherein the expansion medium further comprises B27 and/or N-acetyl-L-cysteine.

28. The method of any one of the preceding embodiments, wherein the mitogenic growth factor is EGF, FGF10 and FGF2, wherein the TGF-beta inhibitor is A83-01, wherein the Wnt agonist comprises R-spondin and CHIR-99021, wherein the cAMP pathway activator is forskolin, wherein the activator of the prostaglandin signalling pathway is PGE2, wherein the BMP inhibitor is noggin.

29. The method of embodiment 28, wherein the final concentration of nicotinamide is about 1 mM to 100 mM, the final concentration of EGF is about 0.1 ng/ml to 500 ng/ml, the final concentration of FGF10 is about 0.1 ng/ml to 500 ng/ml, the final concentration of FGF2 is about 0.1 ng/ml to 500 ng/ml, the final concentration of A83-01 is about 1 nM to 100 μM, the final concentration of R-spondin is about 150 ng/ml to 250 ng/ml, the final concentration of CHIR-99021 is about 10 nM to 500 μM, the final concentration of forskolin is about 20 nM and 50 μM the final concentration of PGE2 is about 10 nM to 500 μM, and the final concentration of noggin is about 50 ng/ml to 150 ng/ml.

30. The method of embodiment 29, wherein the final concentration of nicotinamide is about 10 mM, the final concentration of EGF is about 50 ng/ml, the final concentration of FGF10 is about 10 ng/ml, the final concentration of FGF2 is about 5 ng/ml, the final concentration of A83-01 is about 500 nM, the final concentration of R-spondin is about 200 ng/ml, the final concentration of CHIR-99021 is about 3 μM or 0.3 μM, the final concentration of forskolin is about 1 μM, the final concentration of PGE2 is about 1 μM, and the final concentration of noggin is about 100 ng/ml.

31. The method of any one of preceding embodiments, wherein the epithelial stem cell is part of an organoid or isolated from an organoid, or wherein the population of epithelial stem cells is an organoid, part of an organoid or isolated from an organoid.

32. The method of any one of the preceding embodiments, wherein the epithelial stem cell is a tumour cell or the population of epithelial stem cells are tumour cells.

33. The method of embodiment 32, wherein the tumour is head and neck squamous cell carcinoma.

34. The method of any one of the preceding embodiments, wherein the medium further comprises an Mdm2 agonist.

35. The method of embodiment 34, wherein the Mdm2 agonist is Nutlin-3.

36. The method of any one of the preceding embodiments, wherein the method does not use feeder cells.

37. The method of any one of the preceding embodiments, wherein the cell number increases exponentially, optionally wherein the cell number increases exponentially over 5 passages.

38. The method of any one of the preceding embodiments, wherein the epithelial stem cell or population of epithelial stem cells has been cryopreserved and recovered.

39. The method of any one of the preceding embodiments, wherein the method further comprises the step of cryopreserving and/or recovering epithelial stem cells before or after the step of culturing the cells in the expansion medium.

40. The method of any one of preceding embodiments, wherein the method further comprises a step of culturing the expanded epithelial stem cells in a differentiation medium.

41. The method of any one of the preceding embodiments, wherein the extracellular matrix is a Basement Membrane Extractor Matrigel.

42. The method of any one of the preceding embodiments, wherein the cells are mammalian cells.

43. The method of embodiment 42, wherein the cells are human cells.

44. A method for culturing a mouse epithelial stem cell or a population of mouse epithelial stem cells, wherein the method comprises culturing one or more epithelial stem cells in contact with an extracellular matrix in the presence of an expansion medium, the expansion medium comprising a basal medium and further comprising: one or more mitogenic growth factors, one or more Wnt agonists and nicotinamide; wherein the epithelial stem cells are derived from head or neck tissue.

45. The method of embodiment 44, wherein the mitogenic growth factor is EGF and FGF10, wherein the Wnt agonist comprises R-spondin, and wherein the expansion medium optionally further comprises B27 and/or N acetyl-L-cysteine.

46. The method of embodiment 45, wherein the final concentration of nicotinamide is about 10 mM, the final concentration of EGF is about 50 ng/ml, the final concentration of FGF10 is 10 ng/ml and the final concentration of R-spondin is about 100 ng/ml.

47. The method of any one of the preceding embodiments, wherein the method further comprises culturing for a sufficient number of days to obtain an organoid.

48. The method of embodiment 47, wherein the method comprises culturing the organoid or population of cells for more than 15 passages.

49. The method of embodiments 47 or 48, wherein the organoid grows out from a single cell or small clump of cells.

50. The method of any one of embodiments 47-49, wherein the organoid is cultured for at least 6, 8, 10, 12, 14, 16, 18 or 20 weeks.

51. The method of any one of embodiments embodiment 47-50, wherein the method further comprises a step of isolating and/or cryopreserving the organoid.

52. An expansion medium as defined in any one of embodiments 1-51.

53. An organoid obtainable or obtained by a method of any one of embodiments 1-51.

54. The organoid of embodiment 53, wherein the organoid has a dense structure with a keratinised centre.

55. The organoid of embodiment 54, wherein the keratinised centre is characterised by the presence of tonofilaments and desmosomes.

56. The organoid of any one of embodiments 53-55, wherein peripheral cells are Ki67 and p63 positive and central cells are KRT13 positive.

57. The organoid of any one of embodiments 53-56, wherein the organoid has a normal karyotype.

58. The organoid of embodiment 53, wherein the organoid is tumour organoid.

59. The tumour organoid of embodiment 58, wherein there is an enrichment of the variant allele frequency of detected mutations in the tumour organoid relative to the tumour.

60. The tumour organoid of embodiments 58 or 59, wherein the tumour organoids demonstrate chromosome instability.

61. The tumour organoid of any one of embodiments 58-60, wherein the tumour organoid is a dense or cystic structure.

62. The tumour organoid of any one of embodiments 58-61, wherein the tumour organoid comprises transformed epithelial tumour cells.

63. The tumour organoid of any one of embodiments 58-62, wherein the tumour organoid does not comprise immune, connective tissue or vessel elements.

64. The tumour organoid of any one of embodiments 58-63, wherein the tumour organoid has an increased rate of chromosome segregation errors, including anaphase bridges and/or bi-nucleated cells undergoing multi-polar division

65. The tumour organoid of any one of embodiments 58-64, wherein the tumour organoid displays aneuploidy.

66. The organoid or tumour organoid of any one of embodiments 53-65, wherein the organoid is cryopreserved.

67. A composition comprising an expansion medium according to embodiment 52 and an organoid or tumour organoid according to any one of embodiments 53-65.

68. Use of an organoid of any one of embodiments 53-65 as a model for infection.

69. The use of an organoid according to embodiment 68, wherein the infection is viral infection.

70. The use of an organoid according to embodiment 69, wherein the organoid is infected with Herpes Simplex Virus, optionally wherein the Herpes Simplex Virus is HSV1.

71. The use of an organoid according to embodiment 69, wherein the organoid is infected with Human Papilloma Virus, optionally wherein the Human Papilloma Virus is HPV16.

72. A method of modelling viral infection comprising the step of culturing an epithelial stem cell or a population of epithelial stem cells derived from head or neck tissue in accordance with any one of embodiments 1-51, and further comprising infecting the resultant organoid or population of cells with a virus, optionally wherein the virus is a Herpes Simplex Virus, such as HSV1, or a Human Papilloma Virus, such as HPV16.

73. The method of embodiment 72 wherein infecting comprises producing virus and contacting the cultured population of epithelial stem cells with said virus.

74. The method of embodiments 72 or 73, wherein the method further comprises fractionating the virus supernatant, optionally wherein the viral titer of the fractions is determined.

75. An organoid as defined in any one of embodiments 53-65 for use in diagnostics or medicine, optionally in personalised medicine or diagnostics, or regenerative medicine.

76. A method of treating a disease comprising the step of administering an organoid of any one of embodiments 53-65.

77. Use of an organoid of any one of embodiments 53-65 in the manufacture of a medicament for treating a disease.

78. Use of an organoid of any one of embodiments 53-65 in drug screening, target validation, target discovery, toxicology, toxicology screens or an ex vivo cell/organ model.

79. An organoid of any one of embodiments 53-65 for use in a method to guide personalised therapy.

80. Use of an organoid of any one of embodiments 53-65 in an ex vivo method to predict a clinical outcome.

81. A method for testing the effect of a candidate compound, wherein the method comprises:
the method of any one of embodiments 1-51;
exposing the resultant population of cells or the resultant organoid to one or a library of candidate compounds;
evaluating said expanded organoids for any effects,
identifying the candidate molecule that causes said effects as a potential drug; and optionally
providing said candidate molecule, e.g. as a drug.

82. The method of embodiment 81, wherein the organoid is exposed to radiation in the presence or absence of a candidate compound.

83. The method of embodiments 81 or 82, wherein an evaluated effect is selected from the list comprising: a reduction in, or loss of, proliferation, a morphological change, cell death or a change in gene or protein expression.

84. A method for selecting a treatment regime for a patient, wherein the method comprises the steps of:
optionally obtaining a biopsy from the head or neck tissue of the patient;
culturing the biopsy, a tissue fragment of the biopsy, an epithelial stem cell of the biopsy or a population of epithelial stem cells of the biopsy according to the method of any one of embodiments 1-51 to obtain an organoid or a tumour organoid;
exposing the resultant organoid to a treatment regime, including radiation and/or one or more candidate compounds;
evaluating said organoid for any effects;
identifying the treatment of regime that causes said effects; and
optionally providing said treatment to the patient.

### EXAMPLES

### Example 1—Organoids can be Derived from Healthy Oral Mucosa and Recapitulate Morphological and Functional Characteristics

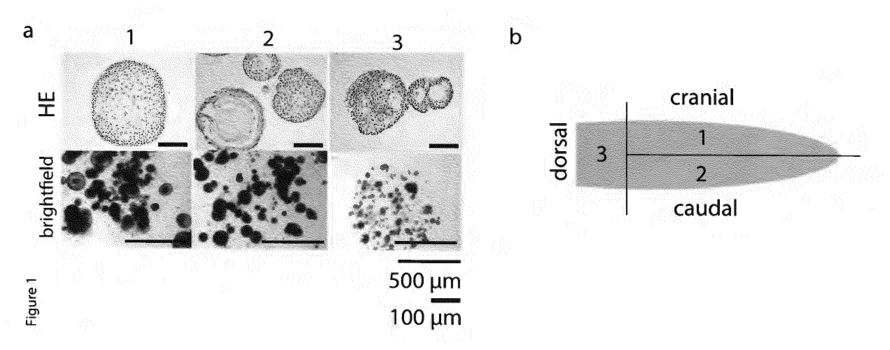

To propagate organoid formation, a range of media compositions was tested as described in previously published protocols for growth support of oral mucosa. Conditions that were successful to grow mouse tongue epithelium (FIG. 1) were refined on human material obtained from surgical resections. A successful culture medium is described in the materials and methods.

The culture method was tested using an expansion medium comprising a basal medium, the mitogenic growth factors EGF, FGF10 and FGF2, the TGF-beta inhibitor A83-01, the Wnt agonists R-spondin and CHIR-99021, cAMP pathway activator forskolin, the activator of the prostaglandin signalling pathway PGE2, and the BMP inhibitor noggin. The method was also tested using the expansion medium without both CHIR-99021 and FGF2, without either of CHIR-99021 and FGF2, or without FGF2. The method was most successful when all components were included in the expansion medium.

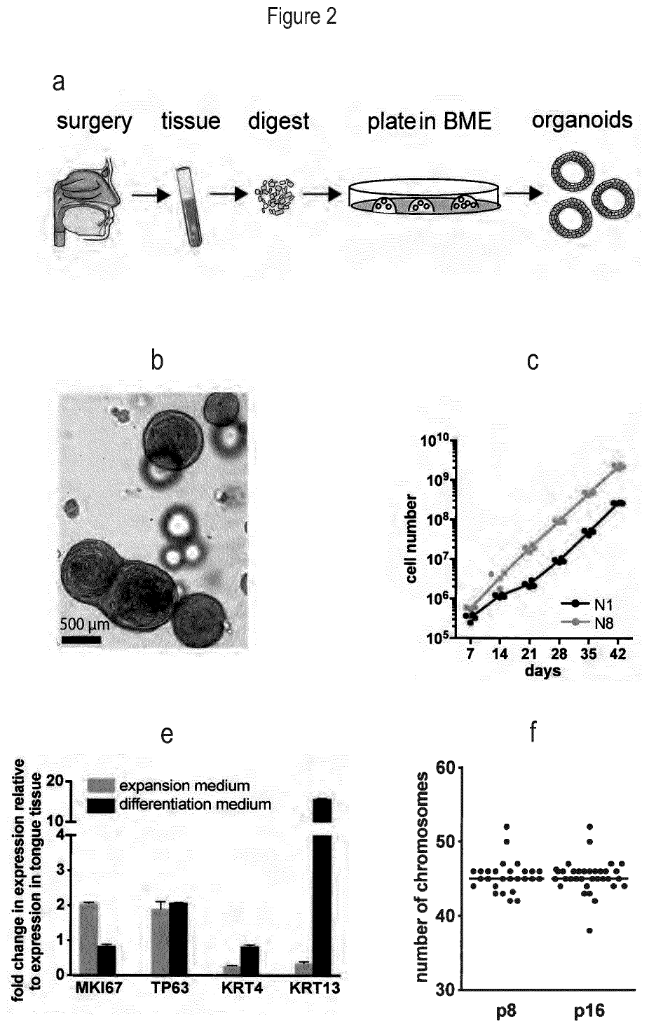

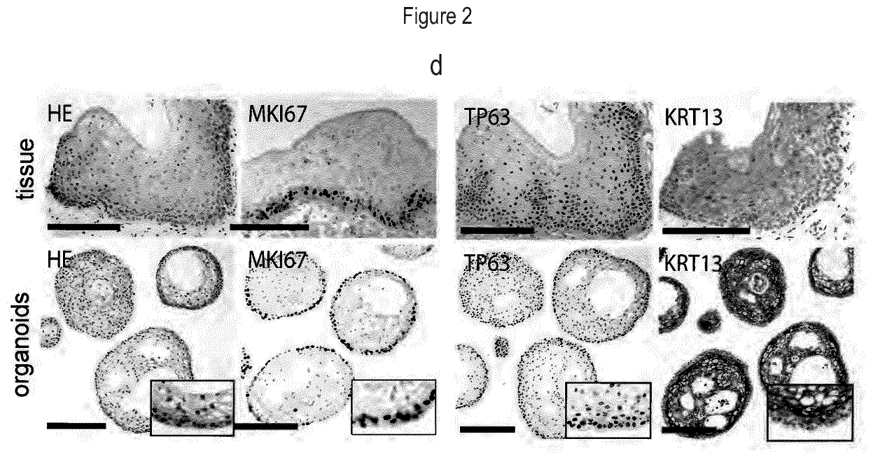

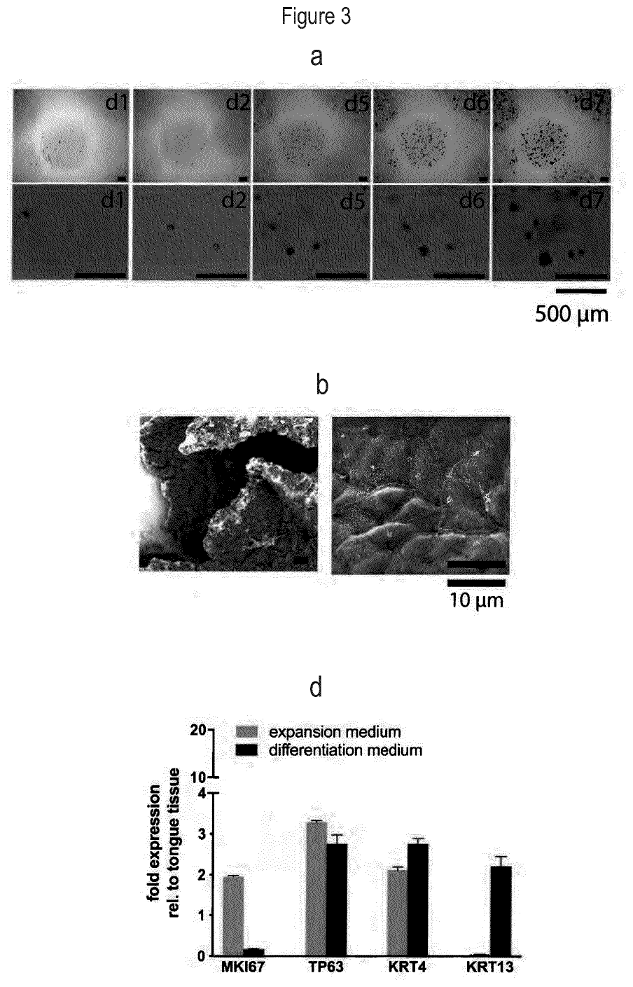

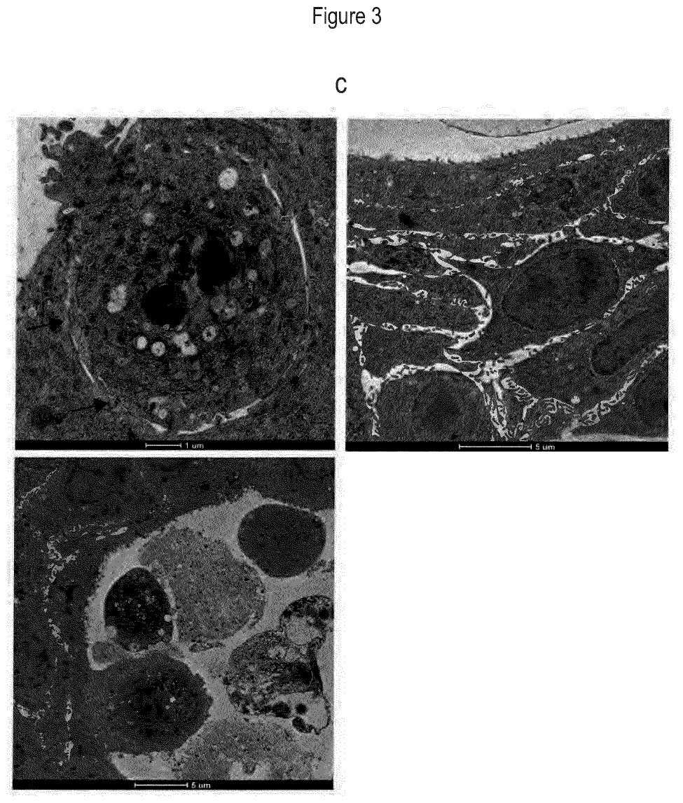

The addition of FGF and a Wnt agonist were shown to be particularly advantageous for head and neck organoid growth. In particular, the FGF2 and GSK-3 inhibitor were surprisingly very important for establishing head and neck organoids. In particular, it was surprising to see that the effect of the GSK-3 inhibitor was greater than the effect of conditioned Wnt medium (given that these are both Wnt agonists acting in the same pathway). In brief, the epithelial layer was micro-dissected from the surgical specimen to remove fat and muscle, digested in 0.125% trypsin, filtered and the resulting cell suspension was then plated in Basement Membrane Extract (BME), a Matrigel equivalent (FIG. 2A). Within the first few days after plating, organoids grew out from single cells or small cell clumps (FIG. 3). Over time, these organoids developed into dense structures, often with keratinized centers (FIG. 2B). The head and neck organoids observed contained stratified (multi-) layers and the epithelium as a whole was polarized, with basal cells on the outside and more differentiated keratinocytes (or even deposited keratin) in the center. Sometimes a lumen was seen, but it was not very common. On average, organoids could be passaged within 10-14 days. After the first passages, organoids typically expanded exponentially, being passaged every ten days with a split ratio of 1:5. Surgical material was obtained from 40 patients, of which 26 (65%) grew out into organoids. Established organoid lines could be expanded long-term (>15 passages) and could be cryopreserved and recovered successfully. The proliferative potential was assessed over 5 passages in expansion medium (FIG. 2C) and unabated exponential growth was observed. Scanning electron microscopy revealed that organoids are composed of tightly connected cells (FIG. 3B). A H&E stain immunohistochemistry of basal cell marker p63 and proliferation marker K167 of paraffin-embedded organoids showed that the organoids recapitulate the tissue of origin. Proliferative Ki-67/P63+ basal cells were located in the external organoid layer touching the basal membrane substitute BME, recapitulating the tissue of origin (FIG. 2D). Differentiation marker KRT13 was detected in cells in the interior of the organoid. Characteristics of keratinocytes such as abundant tonofilaments and desmosomes were observed using transmission electron microscopy (FIG. 3C). Upon withdrawal of growth factors from the medium, differentiation increased, as shown by >45 fold increase in KRT13 expression, and halted proliferation (decreased expression of K167) (FIG. 2E, FIG. 3D). Lastly, the genetic stability of normal tongue epithelium-derived organoid was assessed by metaphase spread analysis and the normal numbers of chromosomes was observed at passage 8 (median 46±0.42, n=26) and at passage 16 (median 45±0.34, n=33) (FIG. 2F).

### Example 2—Oral Mucosa Organoids can be Productively Infected with Herpes Simplex Virus and Human Papilloma Virus

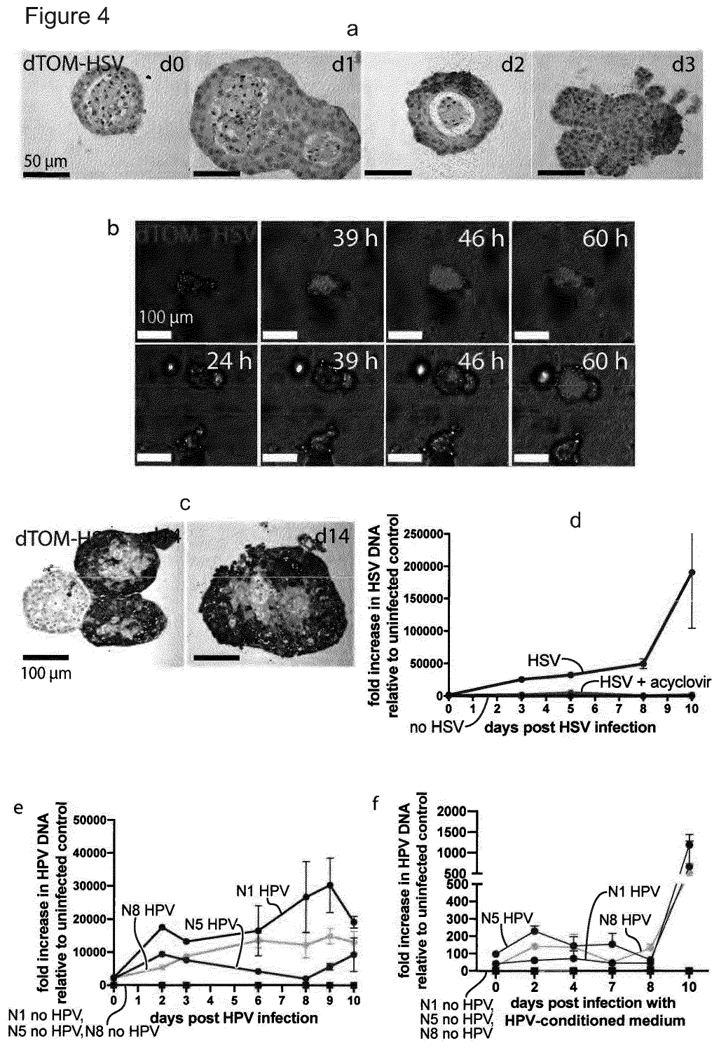

The use of this model to study viral infection was explored with Herpes Simplex Virus type 1 (HSV1), a virus known to infect keratinocytes (17) and to give rise to herpes labialis (cold sores). Using fluorescence microscopy, the infection of organoids with tdTomato labelled HSV was followed (22) (FIG. 4A). Using life imaging, spreading of the infection in organoids was observed two days after initial infection (FIG. 4B). After two weeks in culture, infection had spread throughout entire organoids (FIG. 4C). Infection of organoids resulted in an increase in viral DNA, which could be inhibited by the addition of acyclovir (viral TK inhibitor) (FIG. 4D). This experiment was repeated in organoid lines established from two other donors, where an increase in HSV titer was also observed.

As HPV is known to contribute to oncogenesis of a subset of HNSCC tumors (3,23), HPV16 particles were used to infect oral mucosa organoids (24). Viral replication was quantified by increase in HPV DNA levels (FIG. 4E). Lastly, transfer of filtered supernatant taken 12 days post infection from infected organoids, resulted in re-infection, proving virion production in organoids (FIG. 4F). Taken together, it was concluded that oral mucosa-derived organoids allow infection with HSV1 and HPV16, validating the organoid model as an in vitro model for mucosal pathology.

### Example 3—HNSCC Tumour Organoids Recapitulate Molecular and Morphological Characteristics of the Original Tumour

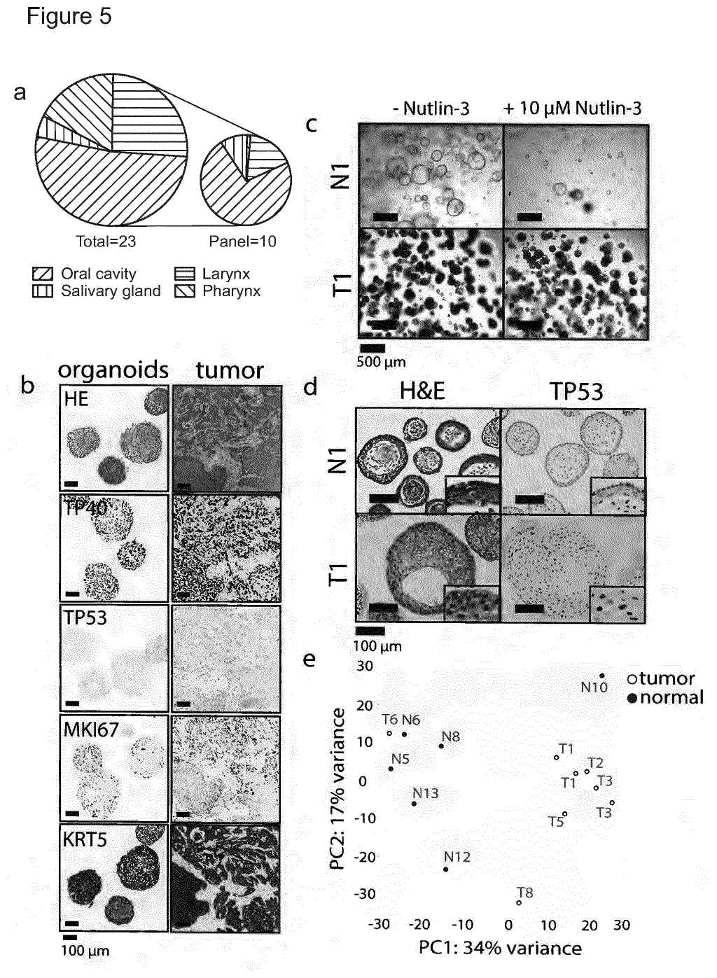

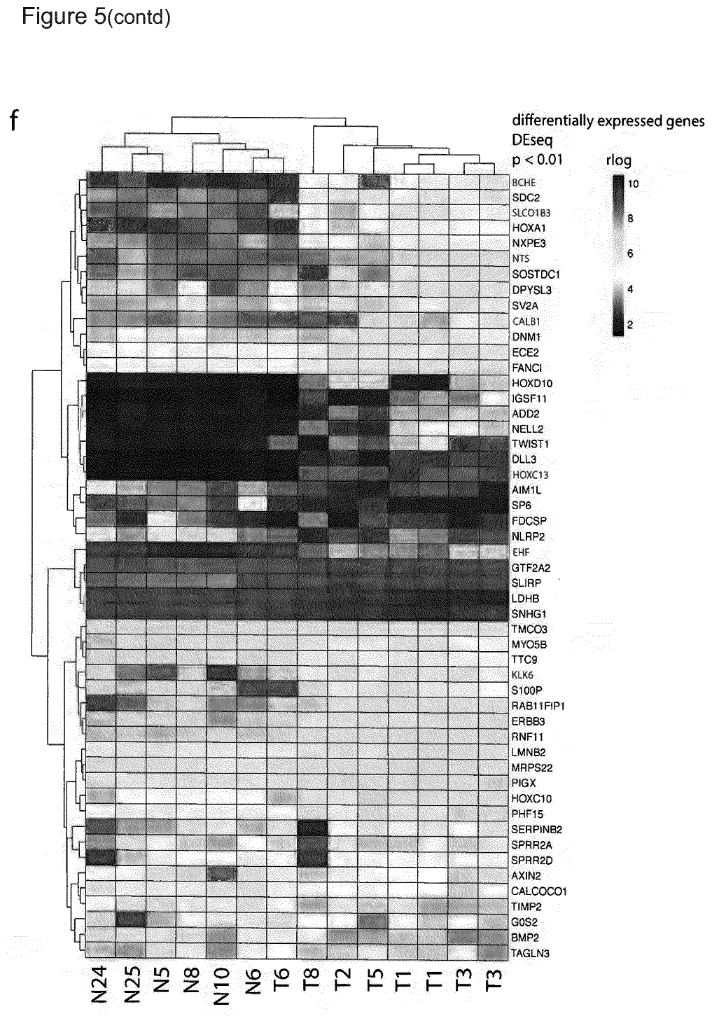

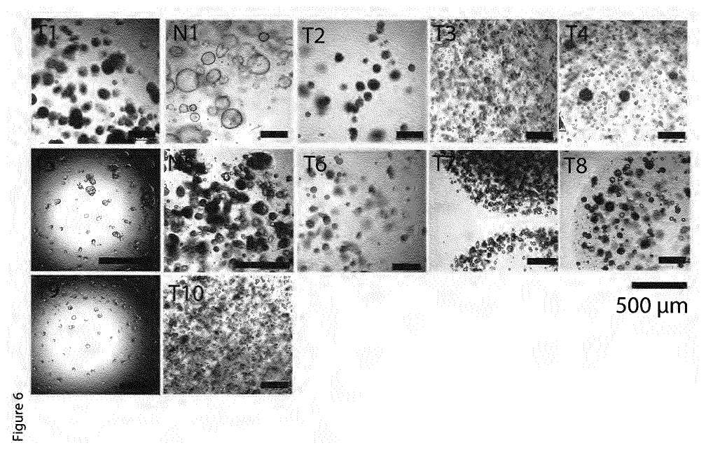

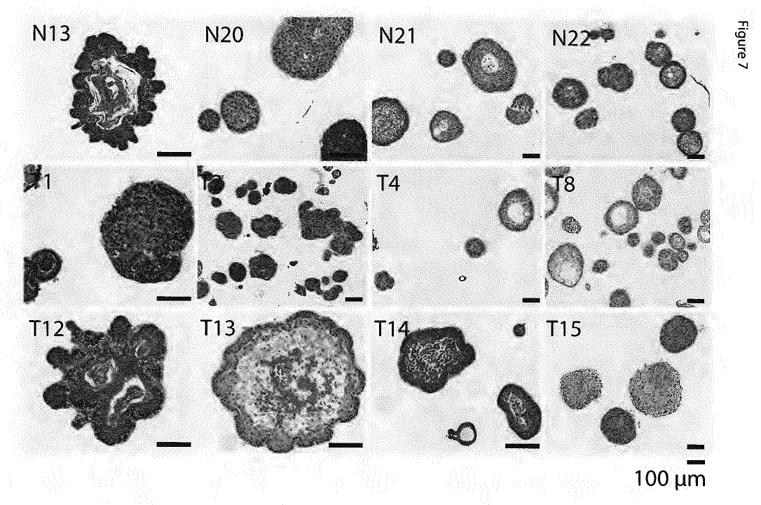

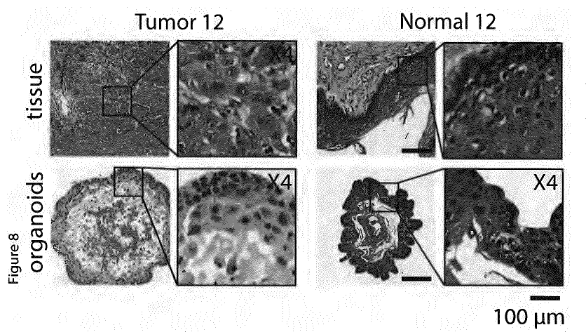

Tumour organoids were successfully established from 23 patients, ranging in age from 48 to 91 (average age at diagnosis: 69). Tumour organoids were established from tumours originating in the oral cavity (floor of mouth, tongue and gingiva/alveolar process), pharynx and larynx (FIG. 5A). Patient clinical data corresponding to established organoid lines can be found in FIG. 19. Of the 23 established tumour organoid lines, 10 were fully characterized molecularly at the date of first submission (data on all others will be added when these become available). These first ten lines are referred to as T1 to T10; the corresponding normal epithelium-derived line of T1 is termed N1 etc. The success rate to establish organoids from tumour tissue was ˜60%. Tumour organoids grew either as dense structures (similar to the normal wildtype epithelial organoids) or as cystic structures (FIGS. 6 and 7). Tumour organoids were generally devoid of a lumen that is visible by brightfield microscopy. Tumour organoids derived from different patients showed different morphologies, as based on brightfield microscopy (FIG. 6) and H&E staining (FIG. 7). Comparison of organoids with the original tumour tissue and adjacent normal epithelium of the same patient revealed tumour-specific histopathological changes that were retained in culture (FIG. 8).

Comparing immunohistochemical stains of the primary tumor specimens with the corresponding tumour organoids revealed that these retained histological characteristics of the epithelial tumor cells (FIG. 5B). However, as described for other adult tissue-derived organoids, organoids/tumour organoids only contain the transformed epithelial tumour cells, but not the immune-, connective tissue- or vessel-elements. This was evident by the keratin staining that marks all cells of the organoids, but only the epithelial component of the tumour section (FIG. 5B).

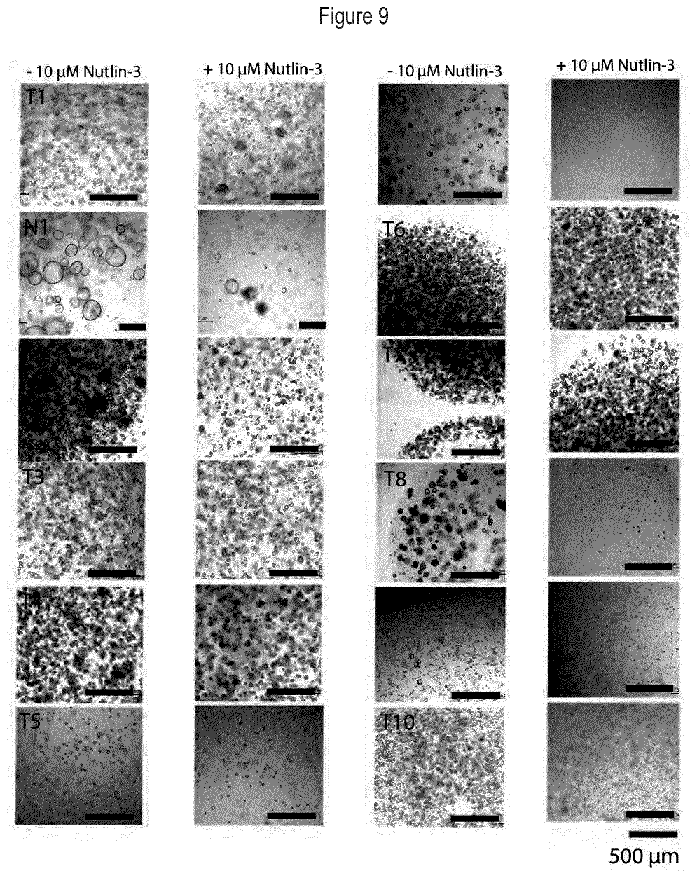

A complication of growing tumour organoids from other carcinoma types has been the gradual overgrowth by normal wildtype organoids (25,26). Yet, to confirm the tumour identity of our tumour organoids, several approaches were taken. Nutlin-3, an Mdm2 agonist, prevents the growth of p53 wildtype cells (27). We observed that all lines except T8, T9 and T10 grew in the presence of Nutlin-3 (FIG. 9), in agreement with the ˜75% of HNSCC 182 carrying inactivating TP53 mutations (28). For example, N1 organoids (p53 wildtype) organoids died in the presence of Nutlin-3, whereas the tumor T1 organoids did not (FIG. 5C). P53 staining on fixed organoid sections confirmed the p53 status (FIG. 5D): p53 staining is caused by accumulation of mutant protein, a clinical parameter to determine the presence of mutant P53 protein (29).

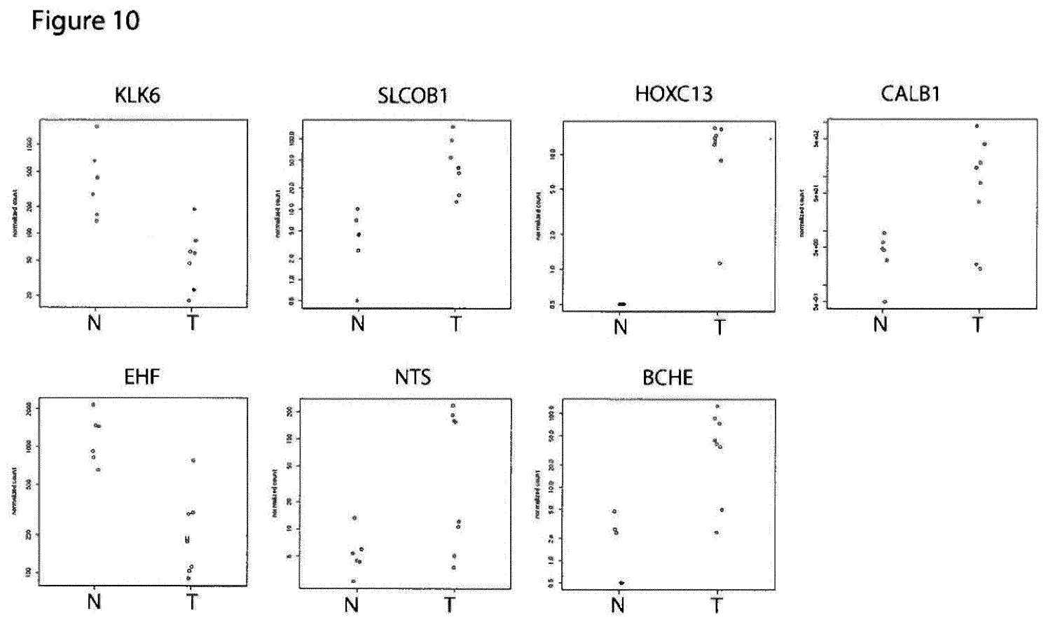

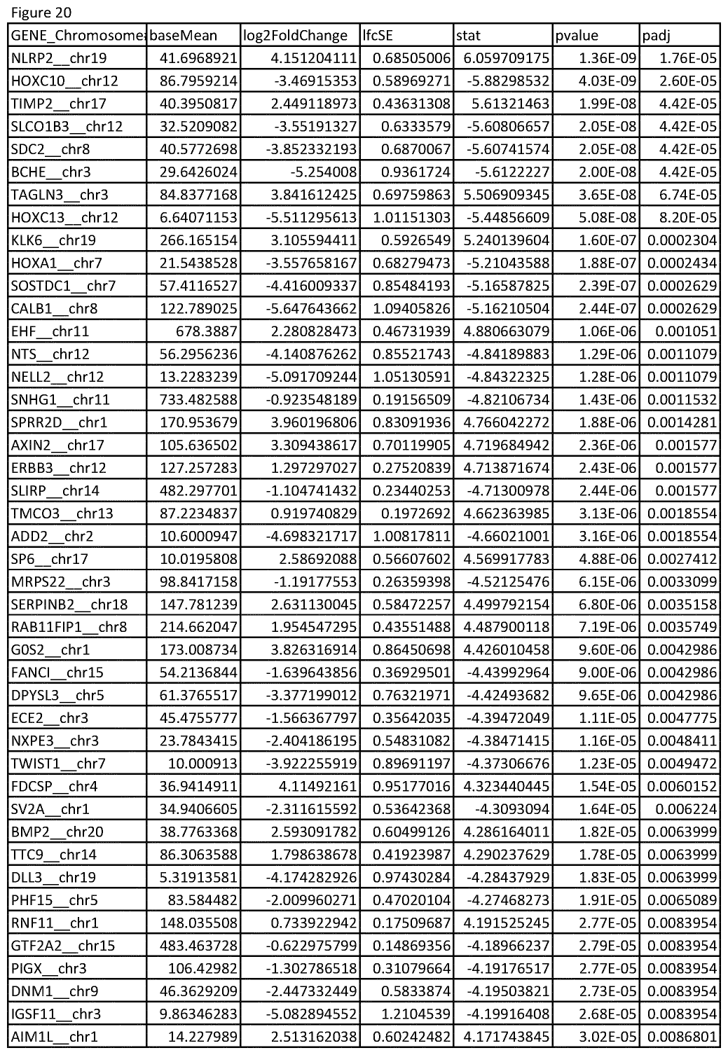

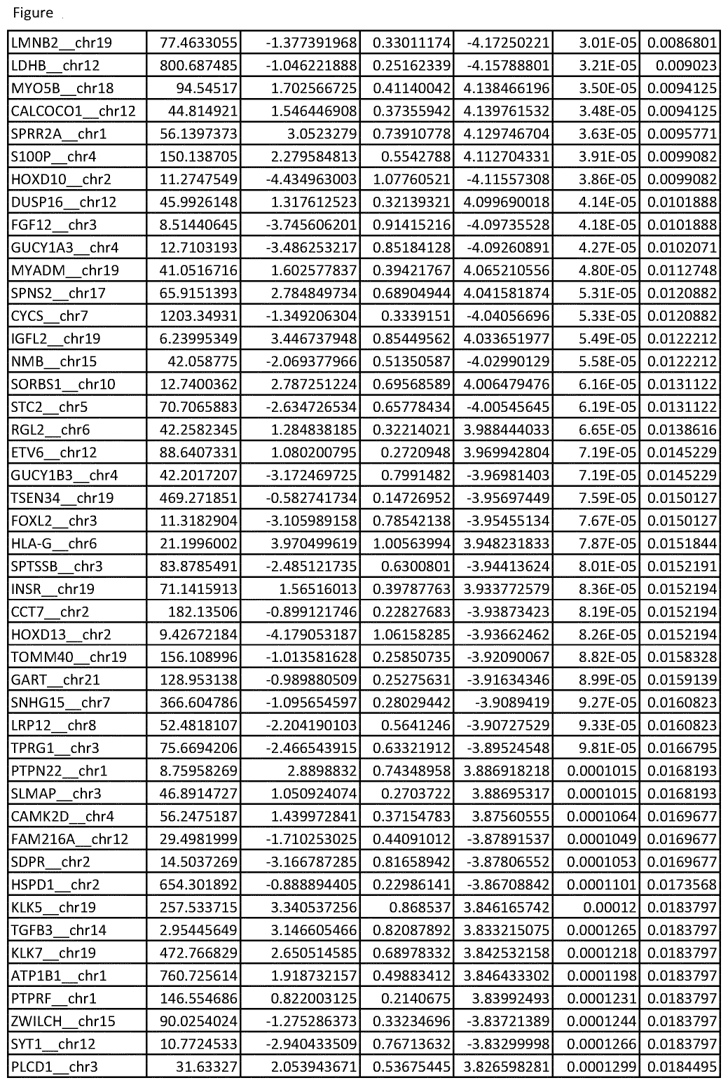

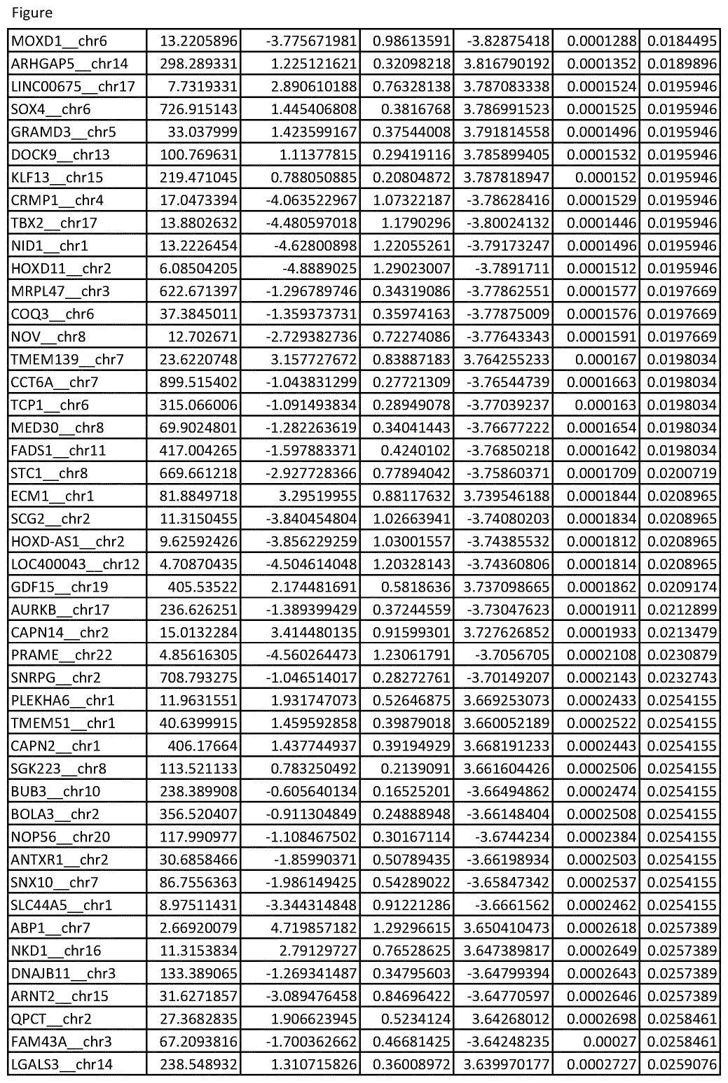

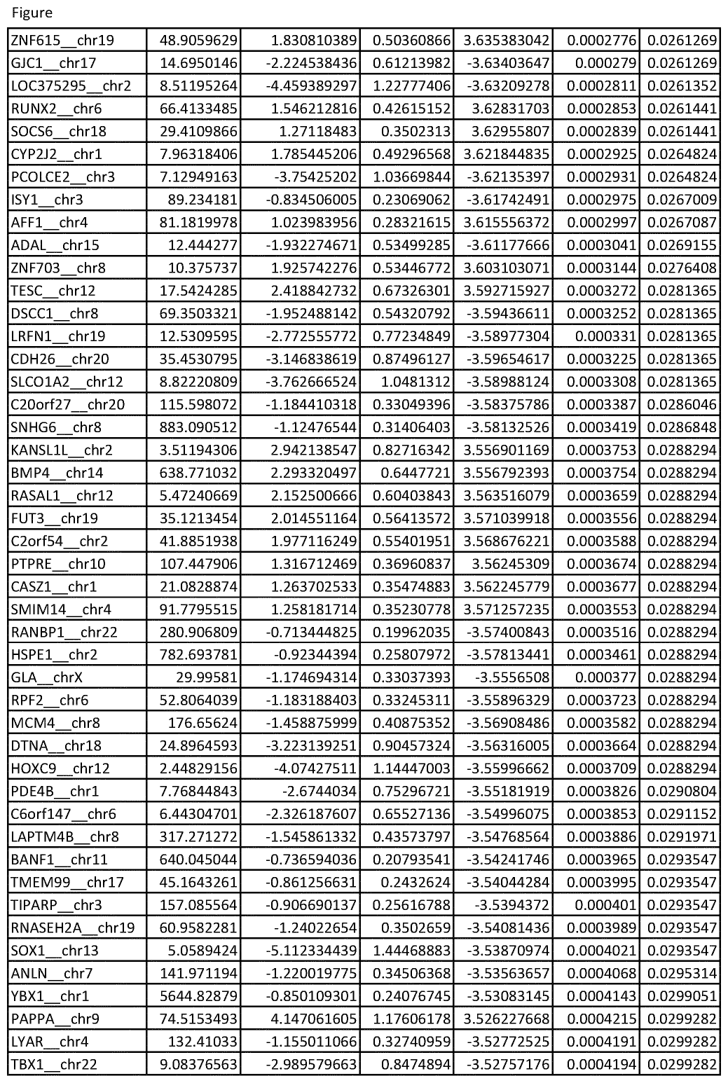

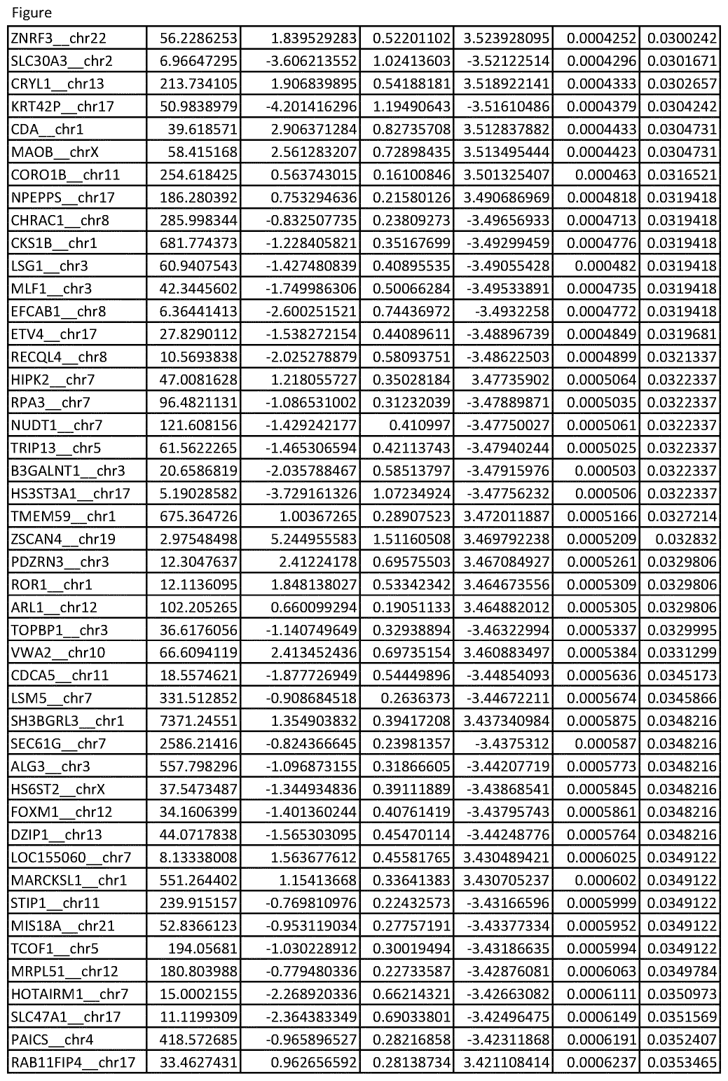

Transcriptome analysis was performed for six normal organoid lines and six tumour organoid lines. As a quality control, two tumor organoid lines (T1 and T3) were sequenced twice in two independent runs. Samples from independent runs clustered together. Principal component analysis revealed segregation of normal and tumor organoids into different clusters, with the exception of tumour organoid line T6 (with confirmed mutations in TP53 and CDKN2A) clustering within the normal wildtype organoid group, and normal line N10 not clustering with the other normal samples (FIG. 5E). DESEQ2 analysis revealed 309 genes to be significantly altered between normal and tumour organoids (p<0.05) (30). Amongst the 51 most differentially expressed genes (p<0.01) we found genes of which the expression is described to be altered in HNSCC including KLK6, SLCO1B3, HOXC13, CALB1, EHF, NTS and BCHE (31-37) (FIG. 5F, FIG. 10). For all 309 differentially expressed genes see FIG. 20.

### Example 4—Tumour Organoids Recapitulate Genetic Alterations Found in HNSCC

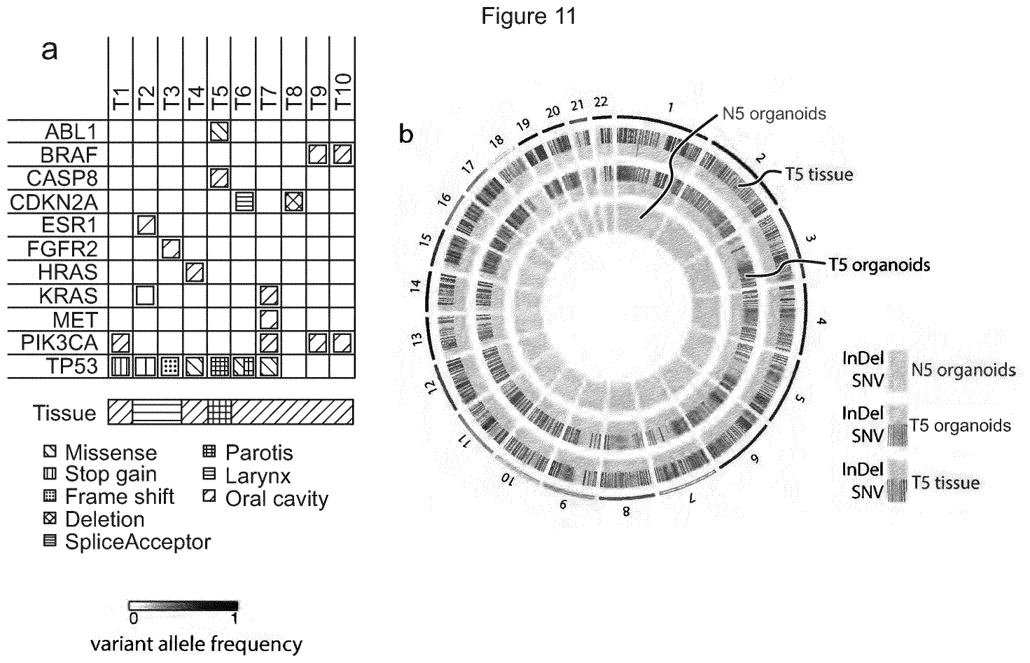

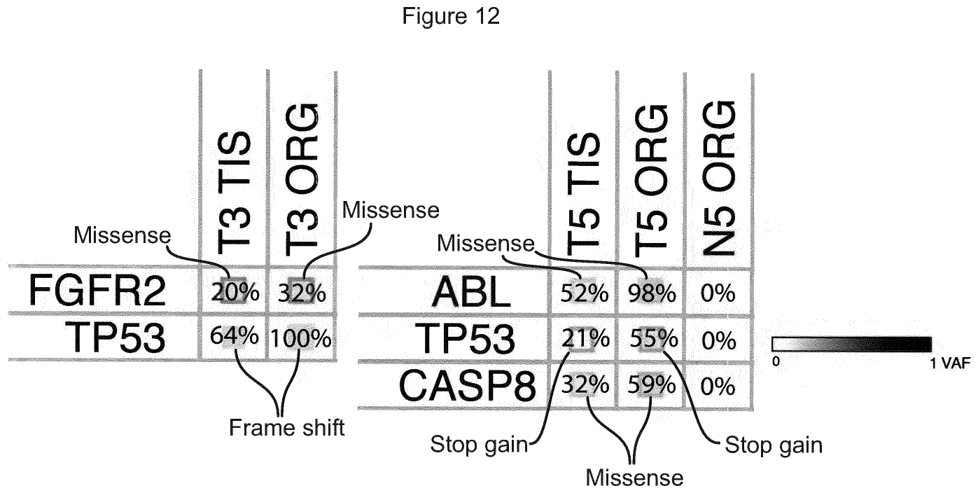

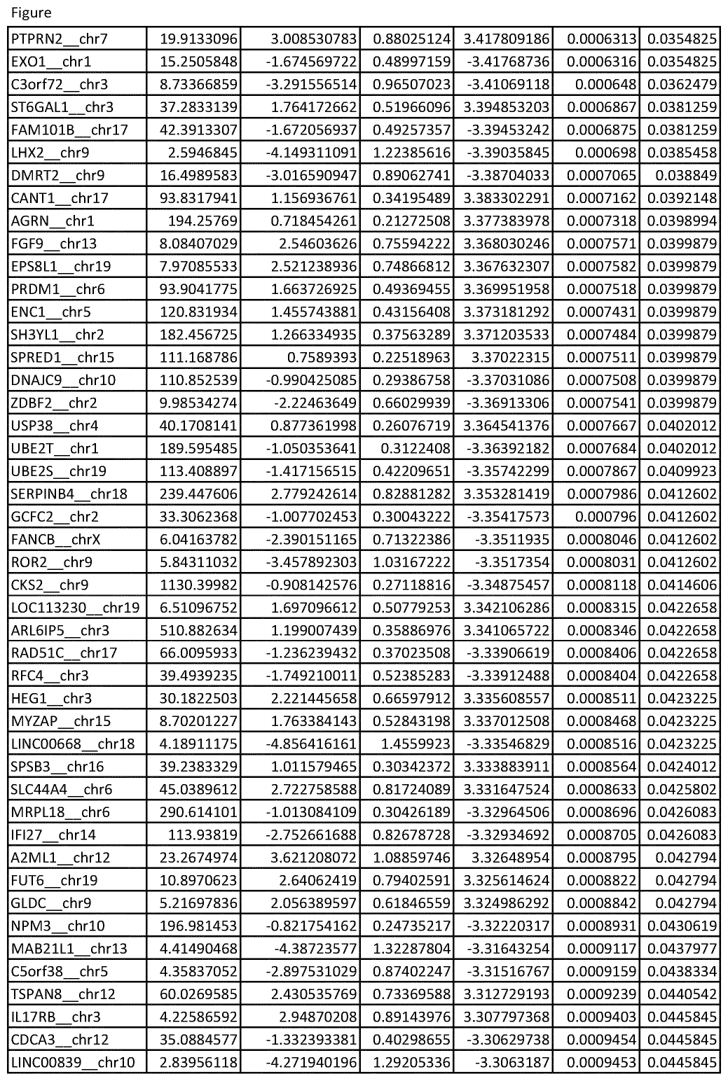

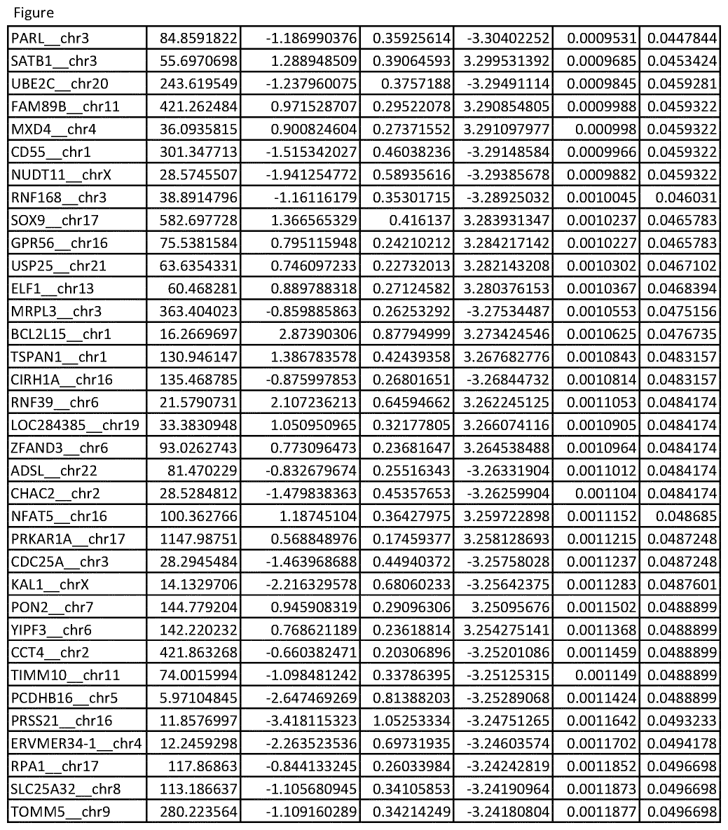

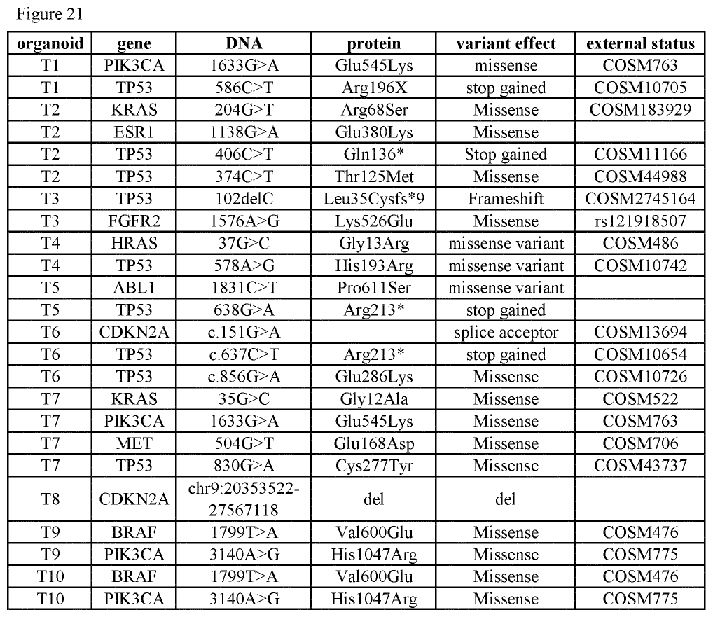

Targeted sequencing (on a panel of 54 oncogenes or tumour suppressors relevant to HNSCC) (n=9) or whole exome sequencing (n=1) was performed on the first ten HNSCC organoid lines. (FIG. 11A and FIG. 21). The most commonly mutated gene TP53 was genetically altered in 7 out of the 10 tumour lines (70%). As expected, no TP53 mutations were detected in the Nutlin3-sensitive lines T8, T9 and T10. PIK3CA was altered in 4 of the 10 tumour organoid lines. BRAF, CDKN2A and KRAS were affected in two cases, and FGFR2, HRAS, ESR1, ABL and MET were each altered in one case. In two cases (T3 and T5), both the original tumour and the tumour organoid line were sequenced (FIG. 12) and an enrichment of the variant allele frequency (VAF) was observed of the detected mutations. Most likely, this is due to the purely epithelial character of tumour organoids, whereas the primary tumour sample also contains blood, vasculature and stromal components in addition to the tumour epithelial cells. In one of these cases (T5) the corresponding normal organoids (N5) were also sequenced and the absence of any of these mutations was confirmed. Subsequently, all single nucleotide variants (SNVs) and small insertions or deletions (Indels) throughout the genome in the tumour and normal tissue were analysed as well as in N5 and T5 organoids (FIG. 5B). Both the tumour tissue and the tumour organoids showed SNVs and Indels that were absent from the normal tissue. Moreover, T5 tumour organoids largely recapitulated the genetic alterations that were detected in the T5 tumour. N5 organoids and normal tissue lacked these genetic alterations, confirming that N5 organoids consisted of normal (non-tumour) cells.

### Example 5—HNSCC-Derived Organoids are Chromosomally Unstable In Vitro

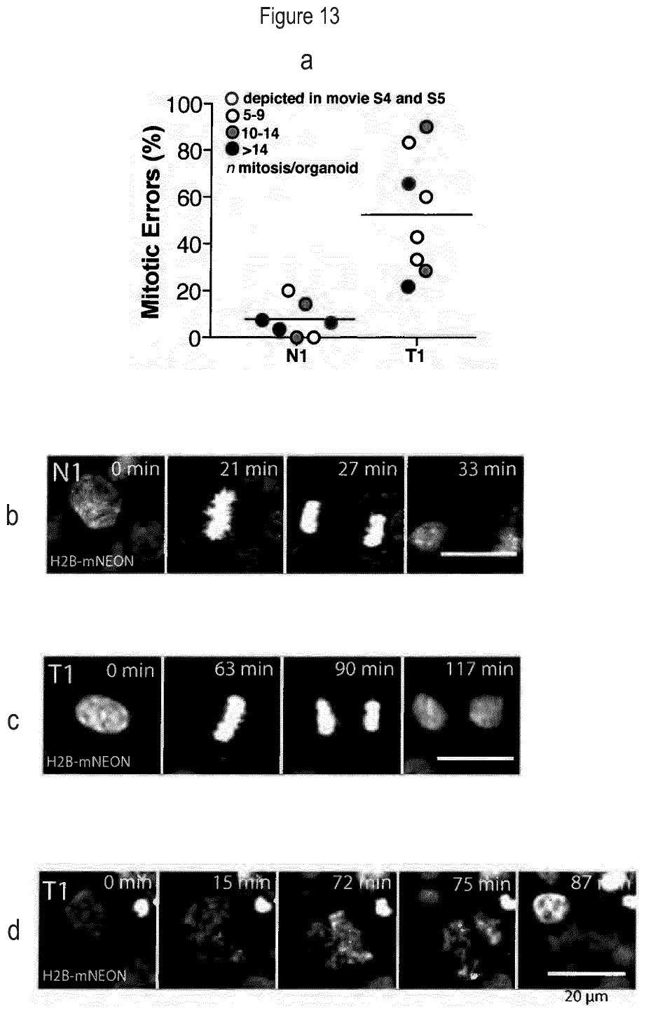

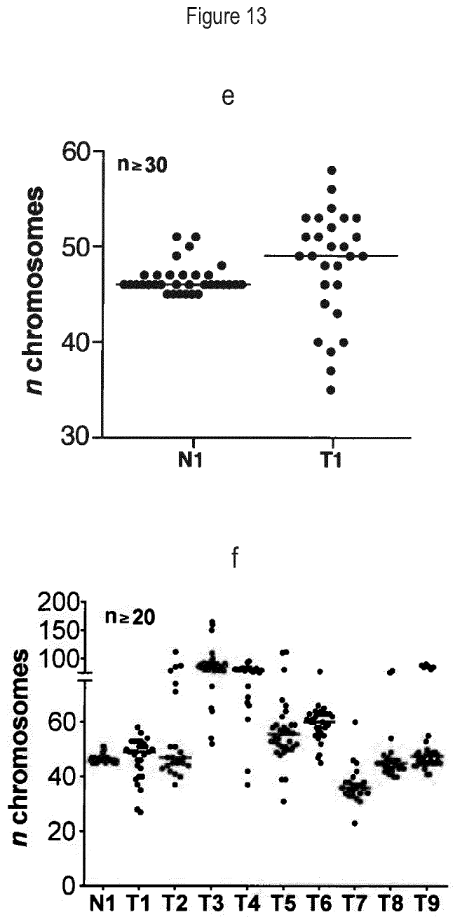

Chromosome miss-segregation underlies the aneuploidies frequently observed in human tumours (38). Increased miss-segregation rates result in the phenotype known as chromosomal instability (CIN), which is commonly observed in cancers, including HNSCC (39). To investigate whether CIN was also present in HNSCC tumour organoids, chromosome segregation was assessed in a matched normal and tumour organoid line. Organoids labelled with Histone2B-mNeon to visualize the chromatin were imaged using a spinning-disc confocal microscope (40). T1 had elevated levels of chromosome segregation errors as compared to its normal counterpart, N1 (FIG. 13A). The majority of cells in N1 showed no signs of chromosome missegregation (FIG. 13B), whereas T1 showed a variety of segregation errors, including anaphase bridges and a bi-nucleated cells undergoing multipolar division (FIGS. 13C and 13D, respectively). It was concluded that T1 has acquired CIN during oncogenic transformation. CIN can result in aneuploidy, by the loss or gain of (parts of) chromosomes (38). In agreement, quantification of the number of chromosomes in cells of N1 and T1 using metaphase spreads revealed that this number was much more variable in T1 than it was in N1 (FIG. 13E). The other tumour organoid lines also carried aberrant chromosome numbers, with T3 and T4 being tetraploid (FIG. 13F).

### Example 6—HNSCC Rumour Organoids Recapitulate Characteristics of HNSCCs Upon Xenotransplantation

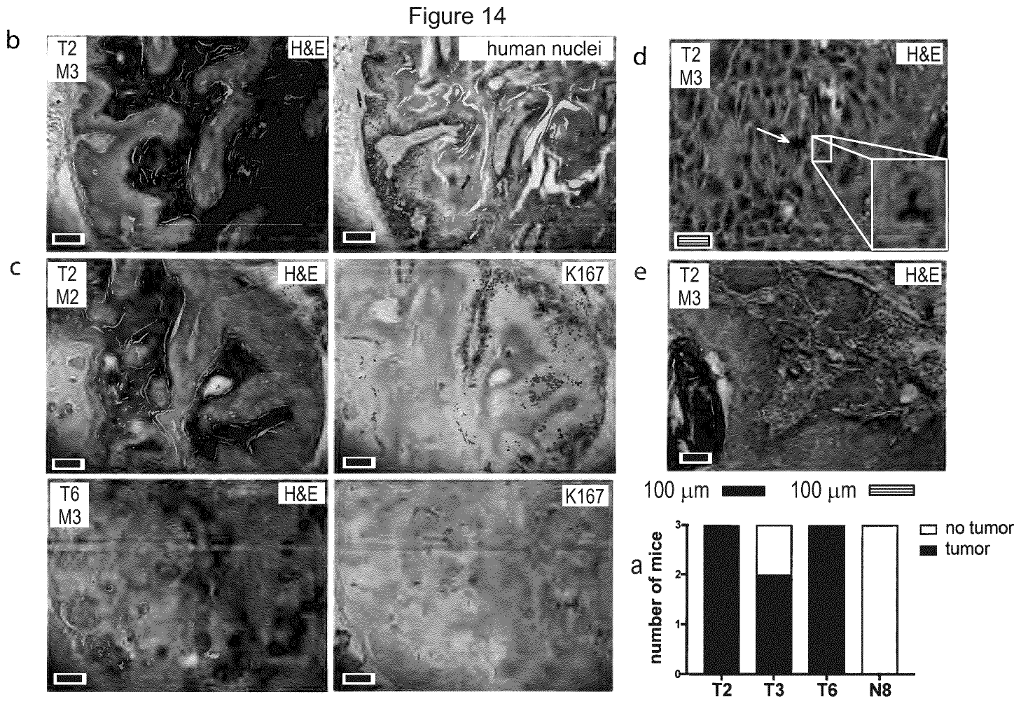

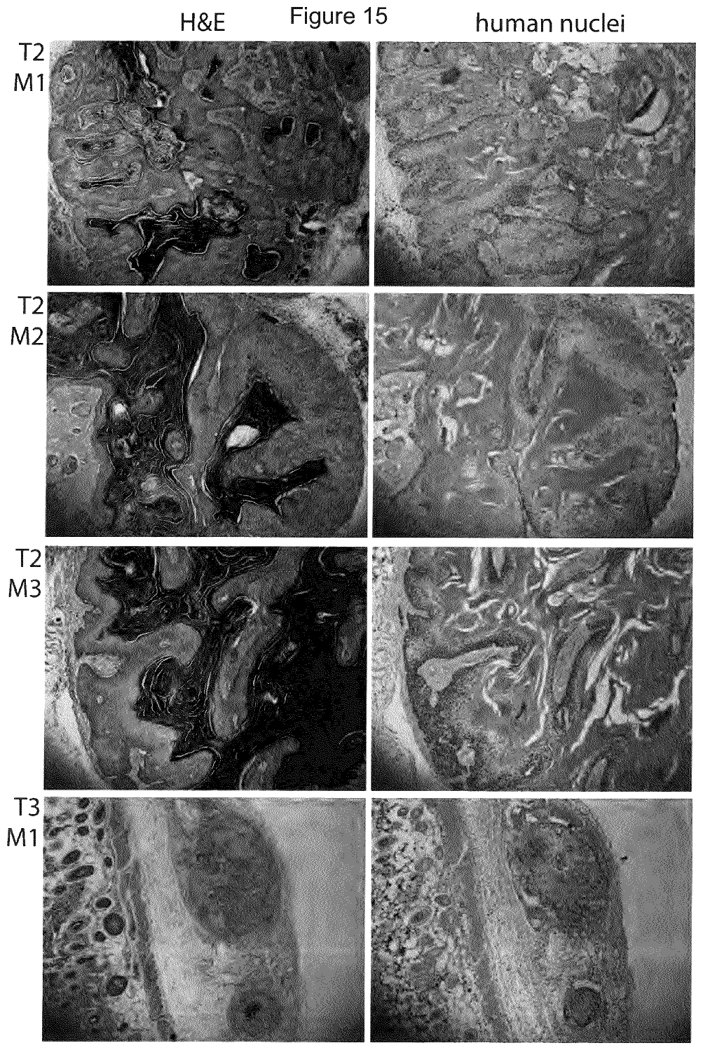

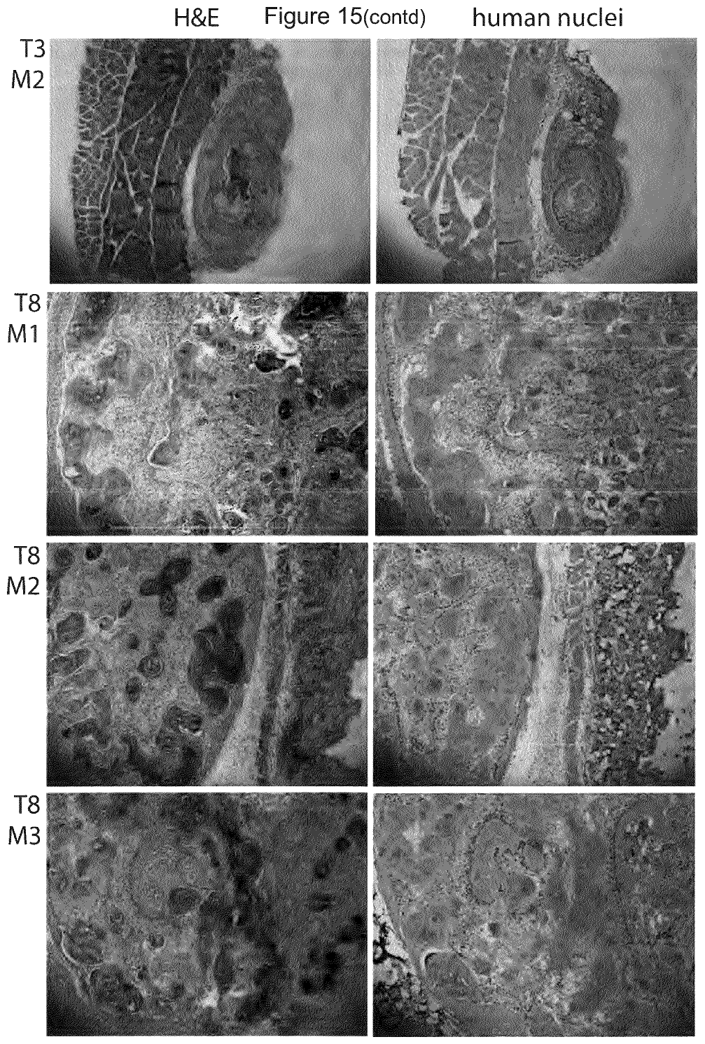

To assess whether tumorigenic potential of the cultured HNSCC cells was retained, tumour organoids were subcutaneously transplanted into mice. Injection of the normal organoids did not result in outgrowth, whereas all three tumour lines yielded macroscopically visible tumours after six weeks in at least two out of three mice (n=3 for each organoid line) (FIG. 14A). For all tumours, H&E staining revealed stratification and keratinization characteristic of HNSCC (FIGS. 14B and 15). Staining for human nuclei showed the human origin of the tumour cells, thus proving the origin of the tumours from the injected tumour organoids. Comparison of proliferating cells, measured by K167 staining, revealed differences in proliferation among the distinct organoid lines (FIG. 14C). These characteristics were retained between mice that were transplanted with the same organoid line (FIG. 15). The tumour cells displayed levels of atypia that were regarded cancerous (assessed by a pathologist). Tripolar mitotic figures and nuclear pleomorphism was observed (FIG. 14D). Moreover, muscle invasion was observed in one case (FIG. 14E). Taken together, this shows that HNSCC organoids retain tumorigenic potential in culture and can form HNSCC with features similar to the parental tumour, upon subcutaneous transplantation into mice.

### Example 7—HNSCC Tumour Organoids as a Platform for Drug-Screening

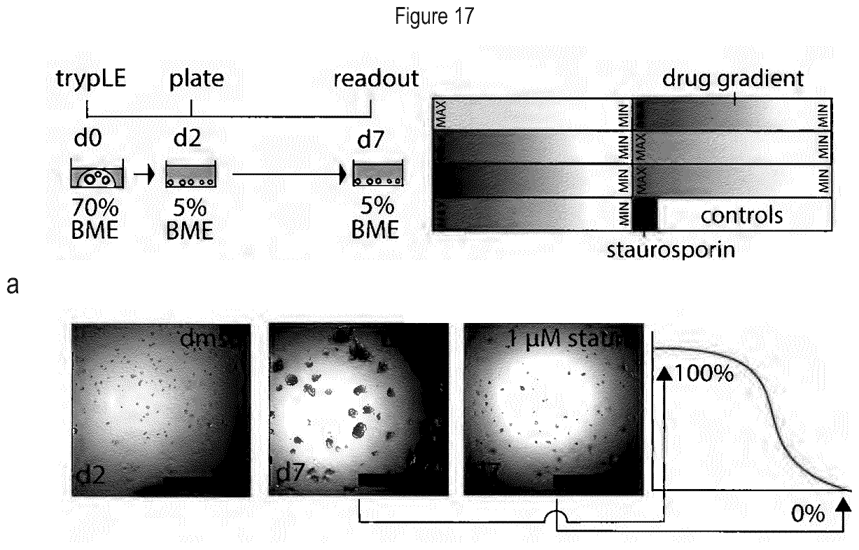

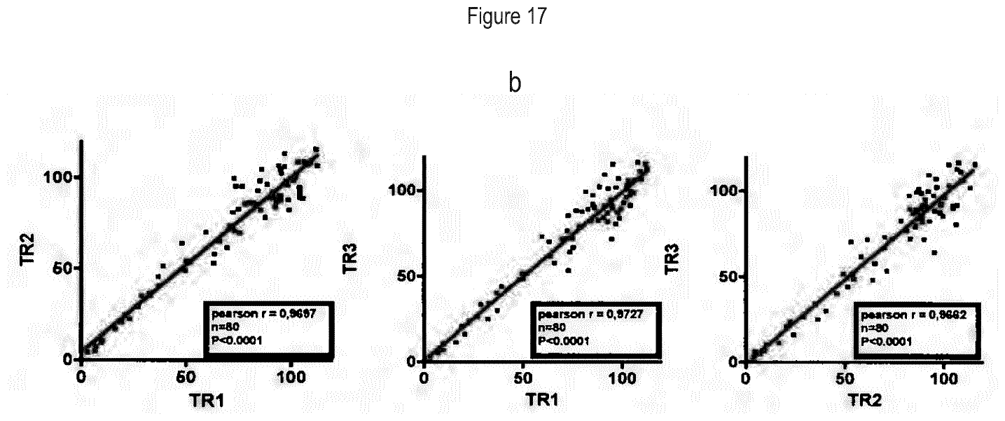

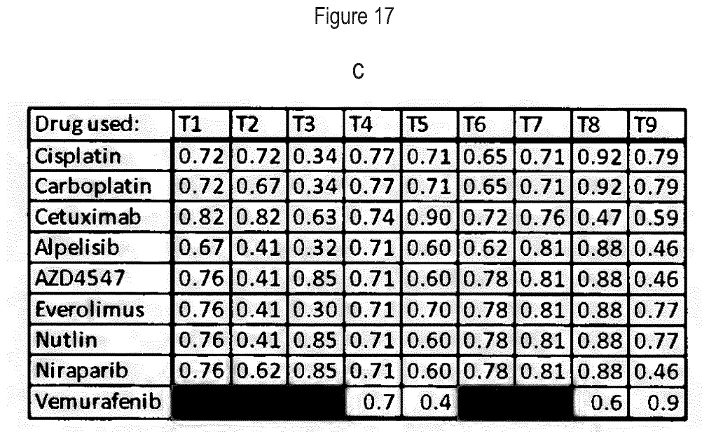

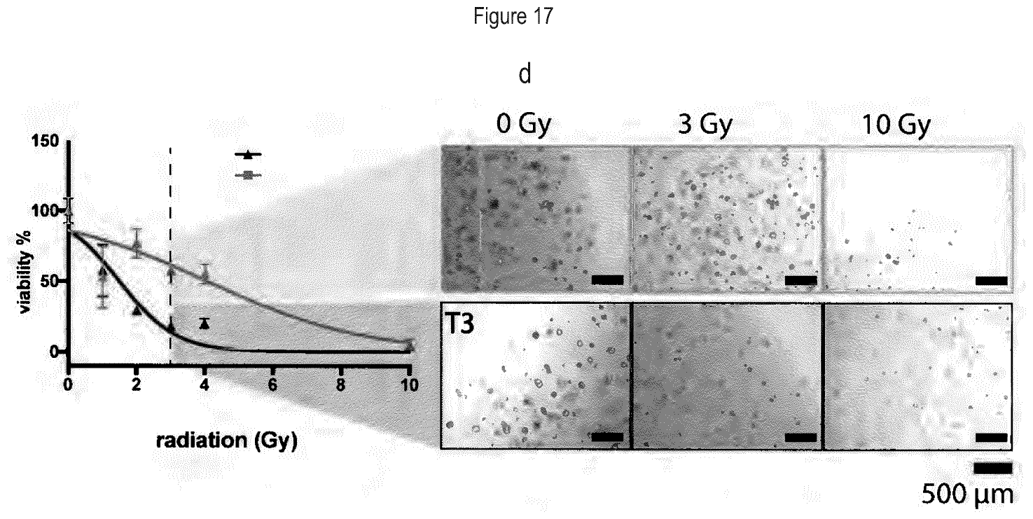

The genetic alterations found in HNSCC are commonly found in other tumour types, and therapies targeting some of these specific mutations exist. Regardless, with the exception of Cetuximab (an anti-EGFR antibody) that is used in treatment of HNSCC, no targeted therapies are currently applied in standard care of these patients (41-44). In recent years, it has become clear that mutation status alone does not provide the required specificity or sensitivity to serve as a predictive marker (45). For this reason, a panel of organoid lines carrying different genetic alterations that are regularly found in HNSCC was tested for their in vitro drug sensitivity (FIG. 17A).

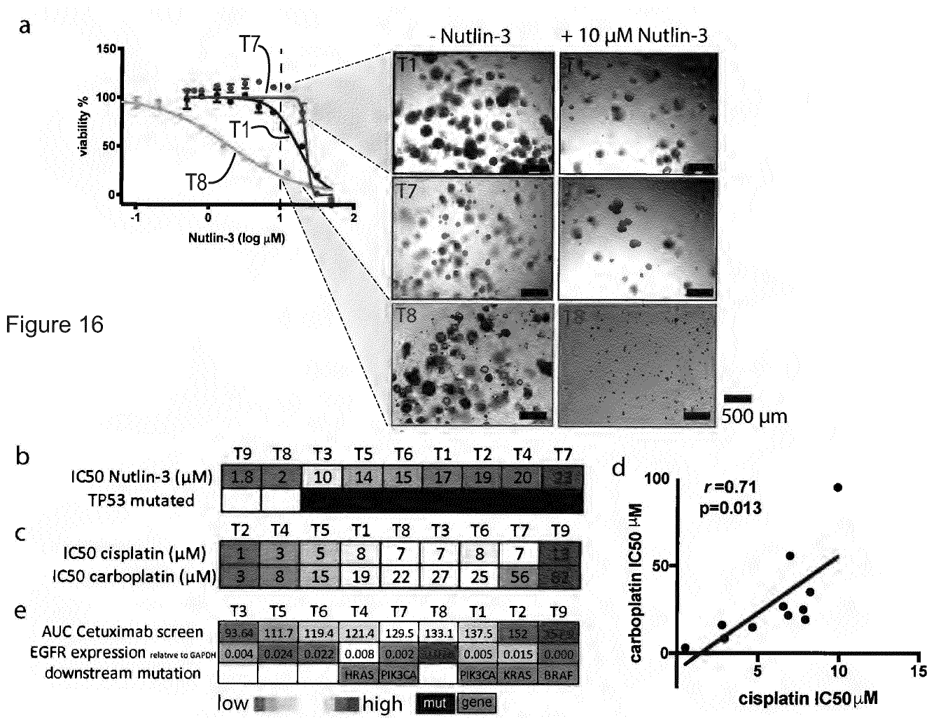

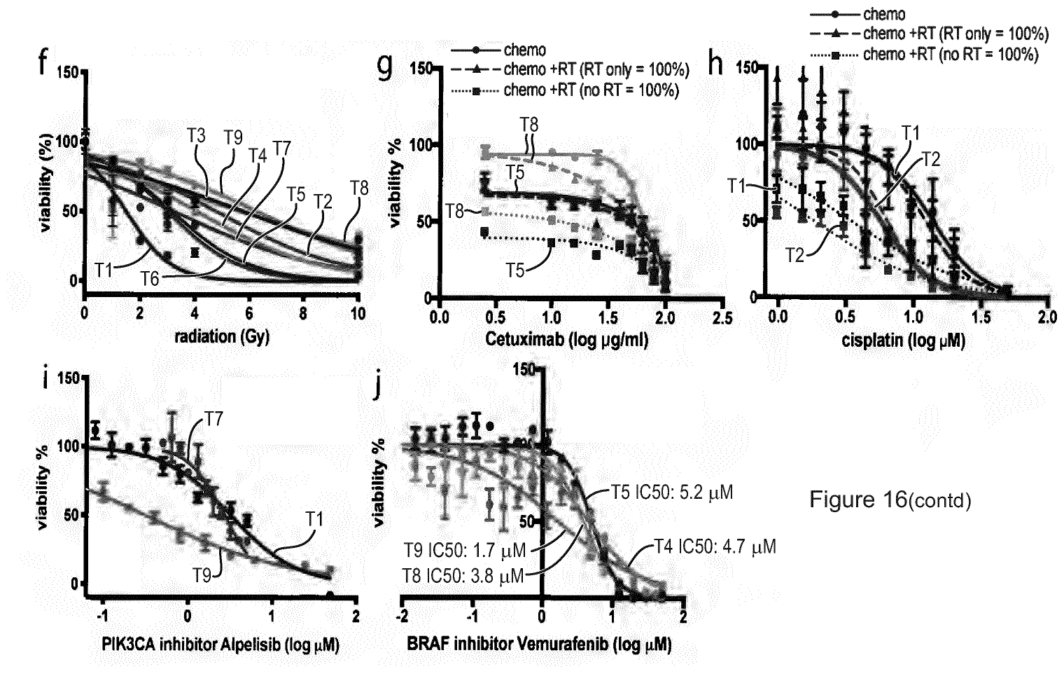

To refine the in vitro drug screening assay, Nutlin-3 treatment was used. Exposure to a concentration series allowed quantitative discrimination between sensitive and non-sensitive lines (FIG. 16A). As expected, TP53 wildtype cells were sensitive to Nutlin-3 exposure, whereas TP53 mutant organoids were not (FIG. 16B). The assay was reproducible (n=3; FIG. S17B). To assure quality of the drug-screen data, a Z factor score (a measure of assay quality) was calculated for each drug screen in this study (Figure S170). Average Z factor score was 0.71 (ranging from 0.30 to 0.92), which is consistent with an experimentally robust assay.

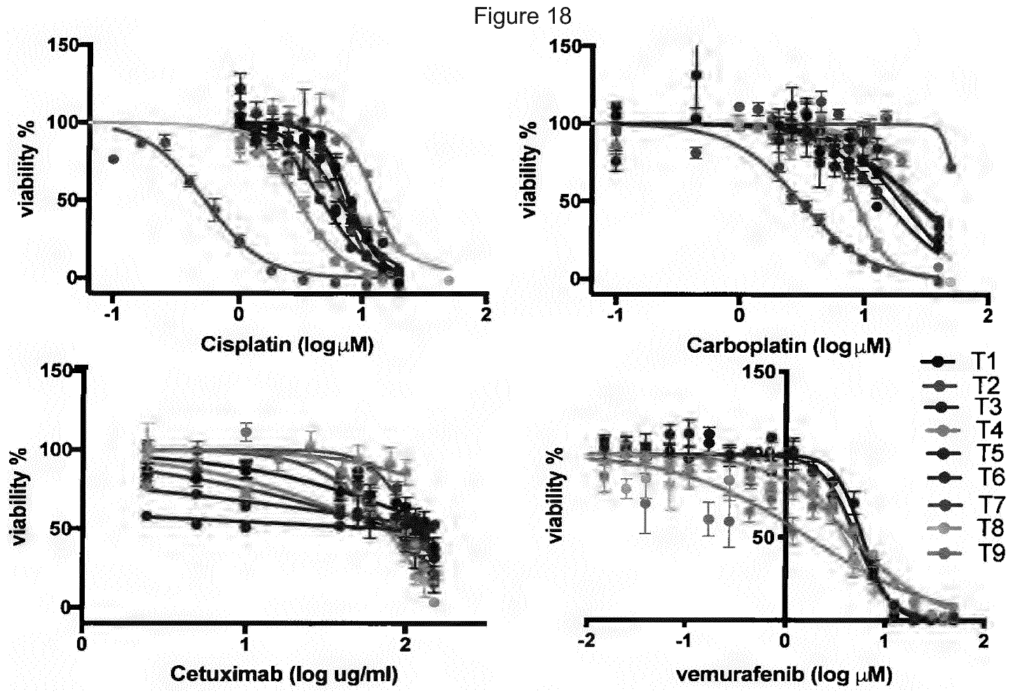

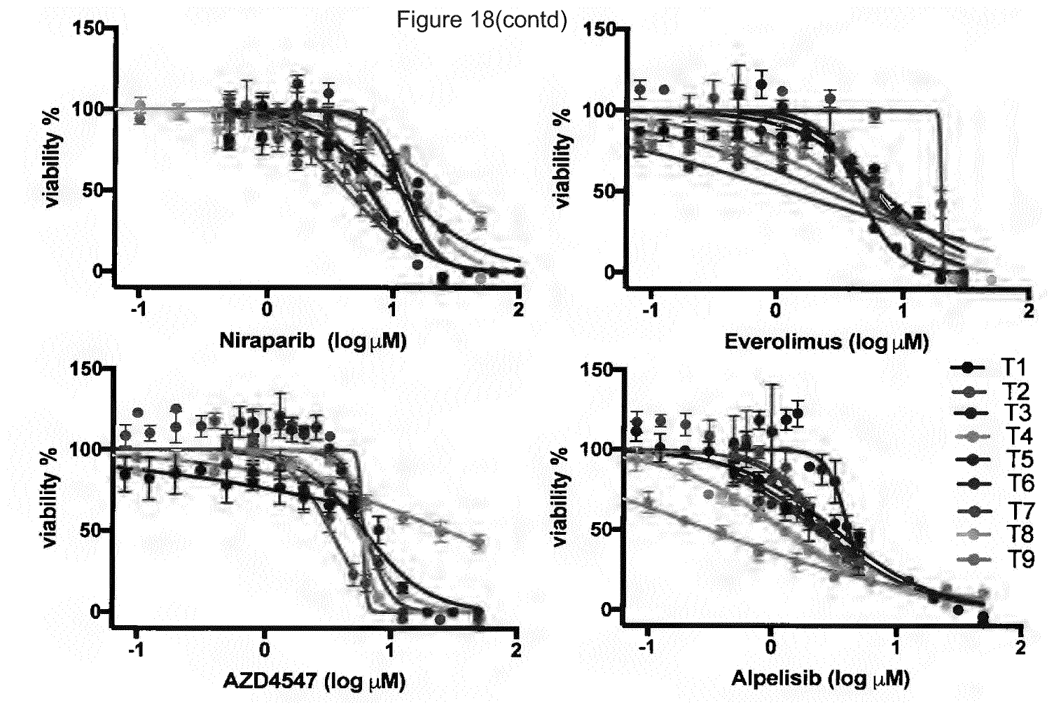

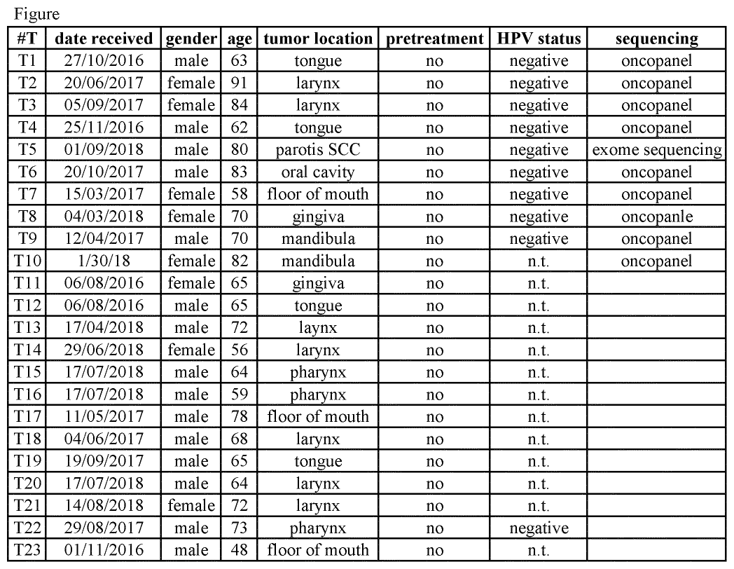

Subsequently, nine fully characterized tumour organoid lines (Tumour organoid line T10 did not survive the robotized drug screening procedure) were exposed to cisplatin, carboplatin and cetuximab, drugs currently used in the treatment of HNSCC patients. Using in vitro concentrations similar to plasma concentrations in HNSCC patients (46), differential sensitivity of the organoids to these compounds was observed. Based on the measured IC50, we made a ranking of the tumour organoid lines tested for cisplatin and carboplatin (FIG. 16C). While IC50 values for cisplatin and carboplatin were different (average IC50cisplatin was 5.93 μM and IC50carboplatin was 29.23 μM), correlation between cisplatin and carboplatin sensitivity was observed (Pearson correlation, r=0.71, p<0.05. FIG. 16D, FIG. 18). This correlation has previously been described in ovarian cancer cells (47). In vitro platinum-DNA adduct formation has shown that both drugs give rise to the same damage, yet cisplatin does so at lower dose (48). Although the mechanism of action is the same (49), it is suggested that cisplatin should be preferred over carboplatin as a radiosensitizer in HNSCC. Indeed, most studies show better overall survival for HNSCC patients treated with cisplatin compared to carboplatin (50). For the lines tested here, none showed a higher sensitivity to carboplatin than to cisplatin.

Organoids were also exposed to the anti EGFR-antibody Cetuximab and differential responses were observed (FIG. 16E, FIG. 18). Recent studies challenge the prognostic value of EGFR overexpression or increased gene copy number for Cetuximab response (45). Therefore, quantitative PCR was performed for all Cetuximab-tested lines. No correlation between EGFR expression and Cetuximab response was observed (FIG. 16E). Organoid lines with mutation downstream of EGFR (PIK3CA, KRAS, HRAS or BRAF) showed decreased Cetuximab sensitivity. This has important implications for patient inclusion for Cetuximab therapy, which currently does not include genetic testing.

Currently, a curative treatment of HNSCC patients with advanced disease consists of chemotherapy combined with radiotherapy (RT) (47). Palliative treatment also includes radiotherapy. Therefore, the sensitivity of the tumour organoids to ionizing radiation was tested. Differential responses between the tumour organoid lines when exposed to RT were consistently observed. This suggested that clinical correlations could be studied (FIG. 16F).

### Example 8—Correlation of In Vitro Organoid Responses with Clinical Responses in Five Patients that Received Radiotherapy

Five of the patients received post-operative RT, which allowed a correlation with organoid sensitivity to RT. Organoid line T3 showed the highest sensitivity to radiotherapy. Indeed, this patient had a lasting response to palliative radiotherapy. The primary tumour of the larynx received 48 Gy in four weeks and showed no signs of growth upon physical examination up to five months later, corresponding with ongoing local control due to radiotherapy. The patient unfortunately succumbed to lung carcinoma five months later. The patient from which T6 was derived, was treated with adjuvant RT following surgery (stage T4aN0 parotid tumour) because of 2 prognostic factors that increase the chance for relapse of disease: macroscopic residual disease and perineural growth, observed in the resected tissue. The patient received a dose of 66 Gy over a period of six weeks at the parotid area. Nine months after completion of RT, the patient showed no signs of relapse or progression upon physical examination, showing a clinically good response to radiotherapy. Organoid lines T1 and T2 were not responsive to RT in the in vitro assay. Indeed, the patients corresponding to these lines showed progressive disease after RT treatment. Patient T1 presented with tongue SCC (stage T2N2b) with extranodal growth and was treated with RT after irradical resection of the primary tumour. The patient received a dose of 66 Gy over the course of 10 weeks. Six months later, the patient complained of pain in the tumour area and loco-regional relapse of disease was eventually diagnosed three months later, leading to death three months after that. Patient T2 presented with a SCC in the larynx (stage T2N0) for which 60 Gy of RT was given after radical resection. Four months later the patient presented with complaints. Five months later, a recurrent tumor was diagnosed that invaded the subglottis and required a complete laryngectomy. Finally, although T8 appeared resistant to RT in the in vitro assay, patient T8 so far did not show relapse after RT treatment. The patient received adjuvant RT after incomplete resection of a gingival SCC (stage T4aN0). A dose of 66 Gy was delivered to the tumour area. Five months later no signs of recurrence were observed upon physical examination. Standard follow up is ongoing. It will be of interest to see if the patient relapses in the coming months, a progression that would be in line with our in vitro findings.

In addition to determining RT sensitivity of the tumour organoids, an in vitro screening assay combining chemotherapy with RT was set up. We exposed organoids to a gradient of chemotherapy in the presence (2 Gy) or absence (0 Gy) of radiation. Previously detected differences in sensitivity for these compounds were confirmed (cetuximab sensitivity T5>T8, cisplatin sensitivity T2>T1) (FIGS. 16G and 16H). Combination therapy resulted in increased cell death at lower doses of chemotherapy than when chemotherapy was given as a single agent. To study the effect of chemotherapy in the presence of RT, organoids treated with chemotherapy and RT were compared with organoids exposed to RT alone (T5 and T8 to Cetuximab, T1 and T2 to Cisplatin (FIGS. 16G and 16H). It seems that in these cases, the effect of chemotherapy itself is not changed by the presence of RT, although the combination treatment results in additional cell death.

Based on mutations detected in this set of HNSCC-derived tumour organoids, the in vitro sensitivity for a range of targeted therapies that are not used in the treatment of HNSCC patients was determined. As T1, T7 and T9 carry activating mutations in PIK3CA, sensitivity to PIK3CA inhibitor Alpelisib was determined for all nine tumour organoid lines eligible for drug screening (FIG. 18). T9 (PIK3CA H1047R, IC50 0.32 μM) showed much higher sensitivity to this compound, as compared to T1 (PIK3CA E545K, IC50 3.19 μM) and T7 PIK3CA E545K, IC50 2.69 μM), that both showed sensitivity comparable to the PIK3CA wildtype lines (FIG. 161). Accordingly, a study linking genetic alterations to Alpelisib responses in patients showed that patients carrying H1047R PIK3CA mutations had a more durable response in the clinic, whereas a negative association was found between E545K mutations and Alpelisib response (51).

BRAF V600E mutations were detected in T9 and T10. Therefore, a panel of tumour organoid lines was tested for sensitivity to vemurafenib, a BRAF inhibitor. FIG. 16J shows the response of T4, T5, T8 and T9 to this compound. As expected, an increased sensitivity of T9 over the other tumour organoid lines tested in this assay was observed.

Finally, the panel of tumour organoids was exposed to the following targeted therapies: PARP-inhibitor Niraparib, mTOR-inhibitor Everolimus and FGFR-inhibitor AZD4547. These compounds were selected based on the genetic alterations described in HNSCC (28). Interestingly, although mutations in PARP, mTOR and FGFR were not detected in these tumour organoid lines, variable sensitivities were observed to these compounds for the 9 organoid lines tested (FIG. 18). This data shows large differences in sensitivity to all compounds tested between different tumour organoid lines, thereby illustrating the potential of this technology to guide personalized therapy in the future.

### Example 9—Optimised Culture Medium for Head and Neck Organoids

To determine the components for the optimised culture medium for head and neck organoids, the media described in Table 1 were compared.

TABLE 1
Culture medium tested for head and neck organoids
(ENR: EGF, Noggin, R-spondin, Nicotinamide).

| Medium | Components |
|---|---|
| A | ENR, A83-01 |
| B | ENR, A83-01, CHIR-99021 |
| C | ENR, A83-01, FGF2 |
| D | ENR, A83-01, FGF2, FGF10 |
| E | ENR, A83-01, PGE2, FSK, FGF2, FGF10, CHIR-99021 |

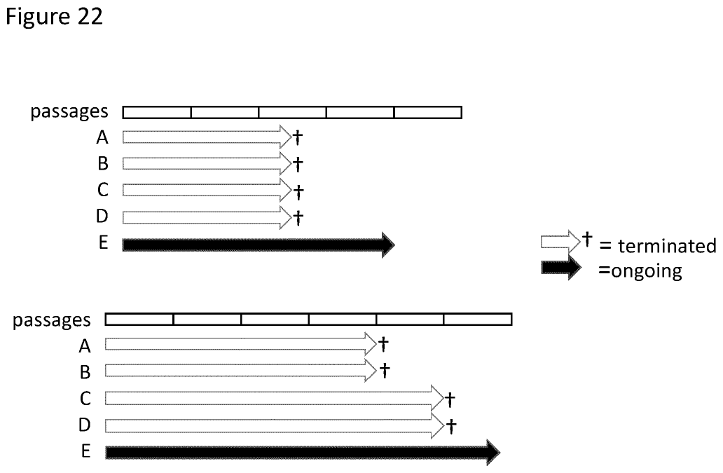

The media that included fewer components (A, B, C and D) supported the culture of head and neck organoids for a few passages. However, the established head and neck organoids could be cultured for longer in medium E (FIG. 22) compared to the media which did not include all of the components of medium E. These data demonstrates that culture medium E is an optimised culture medium for head and neck organoids.

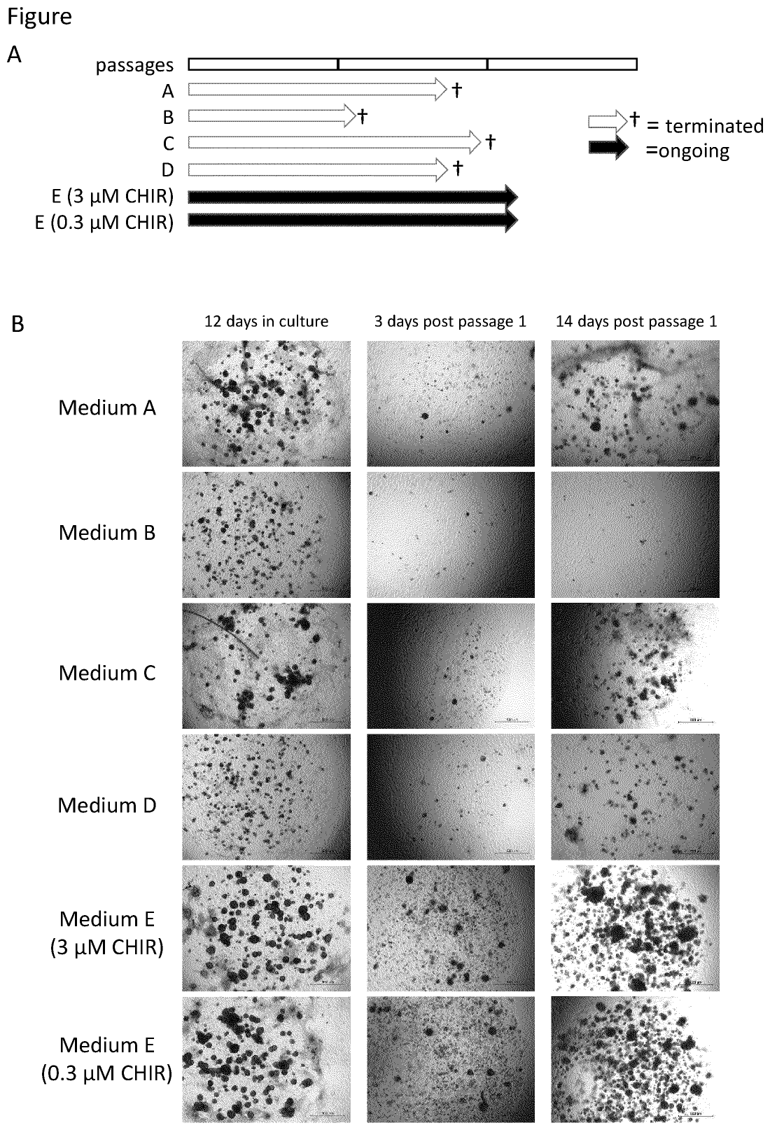

The ability of the media described in Table 1 to support the generation of head and neck organoids from primary tissue was also tested (FIG. 23). Consistent with the observations for the culture of established head and neck organoids described above, only culture medium E supported the generation of organoids from primary tissue that could be cultured long term. Culture medium E supported the generation and culture of head and neck organoids from primary tissue when either 0.3 μM or 3 μM of CHIR-99021 was included in the culture medium. These data demonstrates that culture medium E is an optimised culture medium for head and neck organoids.

### Materials and Methods

### Human Material for Organoid Cultures

The collection of patient data and tissue for the generation and distribution of organoids has been performed according to the guidelines of the European Network of Research Ethics Committees (EUREC) following European, national, and local law. The Biobank Research Ethics Committee of the UMC Utrecht (TCBio) approved the biobanking protocol: 12-093 HUB Cancer according to the UMCU Biobanking Regulation. All donors participating in this study signed informed consent forms and can withdraw their consent at any time, leading to the prompt disposal of their tissue and any derived material, as well as the cessation of data collection. Available organoids will be catalogued at www.hub4organoids.eu and can be requested at info@hub4organoids.eu.

### Tissue Processing

Patient material was collected from pathology material in Advanced DMEM/F12 (Life Technologies, cat. no. 12634-034), supplemented with 1× GlutaMAX (adDMEM/F12; Life Technologies, cat. no. 12634-034), Penicillin-streptomycin (Life Technologies, cat. no. 15140-122) and 10 mM HEPES (Life Technologies, cat. no. 15630-056). This medium was named Advanced DMEM+/+/+. For collection of patient material, 100 μg/ml Primocin (Invivogen, cat. no. ant-pm1) was added to the +/+/+ medium. For normal tissue samples, excess fat or muscle tissue was removed to enrich for epithelial cells and tissue was cut into small fragments. Random pieces of approximately 5 mm3 were stored at −20° C. for DNA isolation. Some pieces were fixed in formalin for histopathological analysis and immunohistochemistry, and the remainder was processed for organoid derivation. Fragments were incubated at 37° C. in 0.125% Trypsin (Sigma, cat. no. T1426) in +/+/+ until digested. Every 10 minutes, the tissue suspension was sheared using 1 ml pipette. Digestion was monitored closely to prevent excess incubation in trypsin. Incubation was performed for a maximum of 60 minutes. When complete, Trypsin was diluted by addition of 10 ml+/+/+. Suspension was strained over a 100 μm EasyStrainer filter (Greiner, cat. no. 542000) and centrifuged at 1000 rpm. The resulting pellet was resuspended in ice-cold 70% 10 mg·ml-1 cold Cultrex growth factor reduced BME type 2 (Trevigen, 3533-010-02) in organoid medium. Droplets of approximately 10 μl were plated on the bottom of pre-heated suspension culture plates (Greiner, cat. no. M9312). After plating, plates were inverted and put at 37° C. for 30 minutes to let the BME solidify. Subsequently, prewarmed organoid medium was added to the plate. For the first week, 10 μM Rho-associated kinase (ROCK) inhibitor Y-27632 (Abmole Bioscience, cat. no. M1817) was added to the medium to aid outgrowth of organoids for the primary tissue. For mouse-derived organoids, tongue tissue was obtained from control mice used under IvD approved projects Subsequent processing of tissue was identical to processing of human tissue.

### Organoid Culture

HNSCC and normal epithelium-derived organoids were grown in Advanced DMEM+/+/+. Organoid medium contained 1× B27 supplement (Life Technologies, cat. no. 17504-044), 1.25 mM N-acetyl-L-cysteine (Sigma-Aldrich, cat. no. A9165), 10 mM Nicotinamide 505 (Sigma-Aldrich, cat. no. N0636), 50 ng/ml human EGF (PeproTech, cat. no. AF-100-15), 500 nM A83-01, 10 ng/ml human FGF10 (PeproTech, cat. no. 100-26), 5 ng/ml human FGF2 (PeproTech, cat. no. 100-18B), 1 μM Prostaglandin E2 (Tocris Bioscience, cat. no. 2296), 3 μM CHIR-99021 (Sigma-Aldrich, cat no. SML1046), 1 μM Forskolin (Bio-Techne (R&D Systems) cat. no. 1099), 4% R-spondin and 4% Noggin (produced via the r-PEX protein expression platform at U-Protein Express BV). Mouse organoids were maintained similar to human organoids, but were grown in +/+/+, containing B27, 25 mM N-acetyl-L-cysteine, 10 mM Nicotinamide, 2% R-spondin, 50 ng/ml EGF and 10 ng/ml FGF10. Organoids were split between 7 and 14 days after initial plating. For passaging, organoids were collected from the plate by disrupting the BME droplets with a P1000, collecting and washing in 10 ml+/+/+. Pellet was resuspended in 1 ml of TrypLE Express (Life Technologies, cat. no. 12605-010) and incubated at 37° C. Digestion was closely monitored and suspension was pipetted up and down every 5 minutes to aid disruption of the organoids. TrypLE digestion was stopped when organoids were disrupted into single cells by adding 10 ml+/+/+. Cells were subsequently resuspended in ice-cold 70% BME in organoid medium and plated at suitable ratios (1:5 to 1:20) to allow efficient outgrowth of new organoids. After splitting, 10 μM Y-27632 was always added to aid outgrowth of organoids from single cells. Medium was changed every 2-3 days and organoids were split once every 1-2 weeks.

### RNA Collection

Organoids were cultured as normal. For differentiation of the organoids (as shown in FIG. 1C), organoids were split to single cells, left to grow one week on organoid medium, and then put on +/+/+ for one week before collection. On the day of collection, organoids were collected from tissue culture plates and washed twice in 10 ml+/+/+. RNA was extracted using RNeasy mini kit (Qiagen, cat. no. 74104) according to protocol. RNA amount was measured using Nanodrop. For quantification of EGFR expression, organoids were split to single cells, left to grow five days on organoid medium, and then put on organoid medium with lower EGF concentration (0.63 ng/ml), but was otherwise left unchanged.

### RNA Sequencing

RNA was extracted using the standard TRIzol (Invitrogen) protocol and used for library preparation and sequencing. RNA was processed as described previously, following the single cell RNA seq protocol of CEL-Seq (61,62), and paired-end sequencing was performed on the Illumina Nextseq500 platform, High Output 2×75 bp run mode. Read 1 was used to identify the Illumina library index and CEL-Seq sample barcode. Read 2 was aligned to the hg19 human RefSeq transcriptome using BWA (63). Reads that mapped equally well to multiple locations were discarded. Around 2 million reads were mapped per sample. Samples with low number of reads were removed. Sample annotation and barcodes can be found on the GEO submission of this data. The remaining samples were normalized and analyzed by the DESeq2 package (30). For visual comparison between samples, regularized log transformed (rlog) values were used.

cDNA Synthesis and Quantitative PCR

For cDNA synthesis, RNA was incubated with 50 μg/ml Oligo(dT) 15 Primer (Promega, cat. no. C1101) in water for 5 minutes at 70° C. Subsequently GoScript Reverse Transcriptase (Promega, cat. no. A5003) was used according to protocol to produce cDNA. qPCR reactions were performed in 384 well format using IQ SYBR green (Bio-Rad, cat. no 1708880) in the presence of 0.67 μM FW and RV primer and cDNA transcribed from 25 ng RNA. For qPCR, samples were incubated for 2 minutes at 95° C. and for 40 cycles at: 15 seconds at 98° C., 15 seconds at 58° C. and 15 seconds at 72° C. Results were calculated by using the ΔΔCt method. Expression was calculated relative to expression in tongue tissue (total RNA, human normal tongue tissue, AmsBio, cat. no. R1234267). Melt peak analysis was performed to assure that primer had no aspecific binding. Primers used were the following:

| Primer | Sequence |
|---|---|
| Human p63 FW | GACAGGAAGGCGGATGAAGATAG |
| Human p63 RV | TGTTTCTGAAGTAAGTGCTGGTGC |
| Human Ki67 FW | GAGGTGTGCAGAAAATCCAAA |
| Human Ki67 RV | CTGTCCCTATGACTTCTGGTTGT |
| Human KRT13 FW | GACCGCCACCATTGAAAACAA |
| Human KRT13 RV | TCCAGGTCAGTCTTAGACAGAG |
| Human KRT4 FW | CTCTTTGAGACCTACCTCAGTGT |
| Human KRT4 RV | GGCTGCTGTGCGTTTGTTG |
| Human EGFR FW | AGGCAGGAGTAACAAGCTCAC |
| Human EGFR RV | ATGAGGACATAACCAGCCACC |
| Human Actin FW | TGCGTGACATTAAGGAGAAG |
| Human Actin RV | TGAAGGTAGTTTCGTGGATG |
| Human GAPDH FW | GGAGCGAGATCCCTCCAAAAT |
| Human GAPDH RV | GGCTGTTGTCATACTTCTCATCG |

### DNA Isolation

DNA was isolated using Reliaprep gDNA tissue miniprep system (Promega, cat. no. A2052) according to protocol. DNA concentration were measured using Nanodrop.

### Growth Rate Analysis

To determine the growth rate of organoid cultures over the course of time, we plated 100.000 single cells, obtained after disrupting organoids with TrypLE, in 50 μl of BME. After one week in culture, all organoids in the well were collected and disrupted into single cells. Cells were counted and total cell number was determined. Counting was performed 4 times. By calculating the number of cells at day 7, which all came from the 100.000 cells plated at day 0, a multiplication factor could be determined for each week. Using this, a theoretical total number of cells could be calculated by multiplying the total cell number of the previous week with the multiplication factor of that week. Subsequently, 100.000 cells of the counted single cells, were plated in 50 μl BME. This procedure was repeated for five weeks, to monitor cell growth over a total of 42 days.

### HSV Infection and Quantification Experiments

For imaging experiments, cells were incubated with 10*10\hat{ }6 PFU HSV-dTomato virus in the culture medium. Virus was a kind gift of Prof. Prashant Desai (John Hopkins University, USA). For DNA quantification, organoids were split using TrypLE. On the third day after splitting, organoids were incubated with 1*10\hat{ }6 PFU HSV-dTomato virus in suspension for 6 hours. After washing with 10 ml+/+/+, organoids were plated (1500 organoids in 20 μl BME per well) in 48 well format. Organoids were kept in organoid medium, with or without 10 μM Acyclovir (Sigma). For DNA collection, the BME drop was collected together with culture medium and added to 10 ml+/+/+ in a 15 ml falcon tube. After centrifugation, medium was removed and pellet was stored at −20 until the day of gDNA extraction. For DNA quantification, qPCR reactions were performed in 384 well format using IQ SYBR green mix (Bio-Rad) in the presence of 0.67 μM FW and RV primer and 2% of total DNA isolated from 1500 organoids. After gDNA extraction, qPCR was performed with the following primers to detect HSV DNA: FW: 5′-ATCAACTTCGACTGGCCCTT-3′ and RV: 5′-CCGTACATGTCGATGTTCAC-3′. PCR program used: 2 minutes at 95° C. and for 40 cycles at: 15 seconds at 98° C., 15 seconds at 60° C. and 15 seconds at 72° C. Increase in DNA content was calculated relative to noninfected wells.

### HPV Infection and Quantification Experiments

HPV16 virions were produced as previously described (24). Upon fractionation of the supernatant containing the virus, fractions with highest titer (as determined by quantitative PCR on HPV DNA) were pooled and subsequently used for infection experiments. Organoids were split using TrypLE and plated at a density of 1500 cells/well, in 20 μl BME drops. After addition of culture media, HPV containing supernatant was added to the wells. During the course of the experiment, medium was refreshed every 2-3 days. DNA isolation and DNA quantification were performed as described for HSV infections, except primers used were FW: ‘5-CTACATGGCATTGGACAGGA-3’ and RV: 5′-GGTCACGTTGCCATTCACTA-3′. For re-infection experiments, supernatant taken from organoids cultured for 12 days after HPV infection was collected and filtered with 0.45 μm pore filter. Subsequently, this was added to the uninfected organoids.

### Next Generation Sequencing

For both NGS and Oncopanel sequencing, gDNA was isolated form organoid cultures as previously described. Oncopanel sample prep and analysis was performed as previously described (64) and sequenced with the mpliSeq Cancer Hotspot Panel V2+(for details: https://www.umcutrecht.nl/getmedia/c39cd469-a4de-4ae9-9a52-0b8ed6761311/CHPv2Plus_NGS.pdf.aspx). For library preparation, SureSelectXT Library Prep Kit was used following the SureSelectXT Target Enrichment System for Illumina Version B.2 protocol. For cluster generation, the library is loaded into a flow cell where fragments are captured on a lawn of surface-bound oligos complementary to the library adapters. Each fragment is then amplified into distinct, clonal clusters through bridge amplification. When cluster generation is complete, the templates are ready for sequencing. Total reads were above 50.000.000 (52<GC %>50). For analysis, we restricted the calling of mutations to the genes checked in the OncoPanel as described above.

Whole exome sequencing data were mapped against human reference genome GRCh37 and variants were called using the IAP pipeline (https://github.com/UMCUGenetics/IAP). To obtain high-quality somatic mutation catalogs, we filtered out variants with evidence in their corresponding normal samples, overlaps with the Single Nucleotide Polymorphism Database v137.b3730, and the variants did not reach our quality measurements (base coverage of 10×, variant allele frequency (VAF) of 0.1, GATK phred-scaled quality score of 100 for base substitutions, 250 for indels and mapping quality (MQ) of 60 for indels). Indels that were present within 100 bp of a called variant in the control were excluded. Only autosomal variants were considered. The scripts used for the filtering are available at: https://github.com/UMCUGenetics/SNVFI, and https://github.com/ToolsVanBox/INDELFI. Non-synonymous mutations (missense mutation, start loss, stop gain, inframe insertion/deletion and frame shift) in the genes checked in the OncoPanel were reported as driver mutations.

### In Vitro Drug Screening

Two days prior to start of the drug screen, organoids were passaged and disrupted into single cells using TrypLE. Single cells were plated in 70% BME in organoid medium as for regular splitting. Two days later, organoids were collected from the BME by addition of 1 mg/ml dispase II (Sigma-Aldrich, cat. no. D4693) to the medium of the organoids. Organoids were incubated for 30 min at 37° C. to digest the BME. Subsequently, organoids were filtered using a 70 mm nylon cell strainer (Falcon), counted and resuspended in 5% BME/growth medium (12.500 organoids/ml) prior plating in 40 μl volume (Multi-drop Combi Reagent Dispenser, Thermo Scientific, cat. no. 5840300) in 384-well plates (Corning, cat. no. 4588).

The drugs were added 1 hour after plating the organoids using the Tecan D300e Digital Dispenser (Tecan). Nutlin-3 (Cayman Chemical, cat. no. 10004372), Niraparib (Selleckchem, cat. no. S2741), AZD4547 (ApeXbio, cat. no. A8250), Everolimus (LC laboratories, cat no. E4040), Vemurafenib (Selleckchem, cat. no. S1267) and Alpelisib (LC laboratories, cat. no A4477) were dissolved in DMSO. Cisplatin (Sigma, cat. no C2210000), Carboplatin (Sigma, cat. no. C2538) and Cetuximab (obtained from hospital pharmacy) were dissolved in PBS containing 0.3% Tween-20, which was required to dispense these drugs using the HP printer. All wells were normalized for solvent used. DMSO percentage never exceeded 1%, PBS/Tween-20 percentage never exceeded 2%. Drug exposure was performed in triplicate for each concentration shown. For a lay-out of the drug screen, see FIG. 18.

120 hours after adding the drugs, ATP levels were measured using the CellTiter-Glo 3D Reagent (Promega, cat. no. G9681) according to the manufacturer's instructions and luminescence was measured using a Spark multimode microplate reader 658 (Tecan). Results were normalized to vehicle (100%) and baseline control (Staurosporin 1 μM), (0%). For each line, when viability did not go above 70% or below 30%, an additional screen was performed for that particular drug with an adjusted dose of this drug for this organoid line. Screen quality was determined by checking Z factor scores for each plate following this formula:

Z factor=1−(3×standard deviation(negative control)+3×standard deviation(positive control))/(average(negative control)−average(positive control))

Drug screens with a Z factor of <0.3 were not used and repeated. Kill curves were produced using GraphPad software and lines were fitted using the option ‘log(inhibitor) vs normalized response-variable slope’.

### Radiation of Organoids

Organoids were disrupted into single cells using TrypLE, and plated at a density of 6000 single cells in 30 μl BME drops in a 48-well plate. Two days later, cells were irradiated. For each radiation dose, a separate plate was used. Plates were sealed air-tight and irradiated with a single fraction of 0-8 Gy using a linear accelerator (Elekta Precise Linear Accelerator 11F49, Elekta, Crawley, United Kingdom). The plates were positioned on top of 2 cm polystyrene and submerged in a 37° C. water bath. After radiation, medium was changed. Four days later, read out was performed as previously described.

### Live-Cell Imaging and Lentiviral Infection

Organoids were infected with lentivirus encoding mNeon-tagged histone 2B and a puromycin resistance cassette (65). After selection, organoids were plated in BME in glass-bottom 96-well plates and mounted on an inverted confocal laser scanning microscope (Leica SP8X), which was continuously held at 37° C. and 5.0% CO2. Over 16-20 h, ˜10H2B-mNeon-expressing organoids were imaged simultaneously in XYZT-mode using a ×40 objective (N.A. 1.1), using minimal amounts of 506 nm laser excitation light from a tunable white light laser. Time interval was approximately 3 min (2:30-3:20 min). Cell divisions were scored, judged and counted manually.

### Transplantations

For all in vivo work, ethical approval was gained prior to the start of this project (HI 17.10.11) Five days before transplantation, organoids were disrupted to single cells and plated as usual. On the day of transplantation, organoids were washed with ice-cold +/+/+ until all BME was removed. Next, organoids were disrupted into single cells and resuspended in 50% BME/organoid medium at a density of 33.33 million cells per ml. 2.5 million cells were subcutaneously injected in NOD.Cg-Prkdc^{scid}II2rg^{tm1Wjl}/SzJ mice, between 6 and 12 weeks of age. Six weeks after injection, mice were sacrificed by cervical dissociation and tumours were excised and fixed overnight in 4% formaldehyde.

### Immunohistochemistry

Tissues or organoids were fixed in 4% paraformaldehyde overnight, dehydrated and embedded in paraffin. Sections were subjected to H&E as well as immunohistochemical staining. The following primary antibodies were used for immunohistochemical staining on organoids and primary tissue:

| | | Order | Host | | Lot | | |
|---|---|---|---|---|---|---|---|
| Protein | Supplier | number | Species | Clone | number | Dilution | Pretreatment |
| KRT5 | Novocastra | NCL-L-CK5 | Mouse | XM26 | 6027941 | Ventana | CC1 24′ |
| | | | | | | 1:200 | (EDTA) |
| | | | | | | | Ventana |
| MKI67 | Monosan | MONX10293 | Mouse | MM1 | 10293 | 1:2000 | Citrate |
| | | | | | | | autoclave |
| TP40 | Abcam | ab172731 | Rabbit | BC28 | GR322490- | Ventana | CC1 48′/ |
| | | | | | 1 | 1:50 | 32′ AB |
| P53 | DAKO | M7001 | Mouse | DO-7 | 95381 | Ventana | CC1 24′ |
| | | | | | | 1:6000 | (EDTA) |
| | | | | | | | Ventana |
| P63 | Abcam | AB735 | Mouse | 4AB | AB735 | 1:800 | Citrate |
| Human | Millipore | MAB1281 | Mouse | 235-1 | 1281 | 1:1000 | Citrate |
| nuclei | | | | | | | autoclave |
| KRT13 | Progen | 10523 | Mouse | 1C7 | 10523 | 1:100 | Citrate |
| dTOM | Rockland | 600-401-379 | Rabbit | | | 1:1000 | Citrate |

Staining for TP40, TP53, MKI67 and KRT5 on the tumoroids (FIG. 3) were performed at the pathology department of the UMCU.

### Karyotyping

Two days after splitting, organoids were treated with 0.1 μg ml-1 Colcemid (Gibco 15212012) for 17 h in organoid medium. After that, organoids were disrupted into single cells using TrypLE and processed as previously described (66). Metaphase spreads were mounted with DAPI-containing vectashield (Vector laboratories, cat. no. H-1200) and imaged on a DM6000 Leica microscope.

### Scanning Electron Microscopy (SEM)

Organoids were collected and BME was removed using Cell Recovery Solution (Corning). To fix organoids, 1 ml of 1% (v/v) glutaraldehyde (Sigma-Aldrich, G5882) in PBS was added. Following o/n fixation at 4° C., organoids were transferred onto 12 mm coverslips (Corning, cat. no. 354085). Samples were dehydrated by consecutive 10 min incubations in 2 ml of 10% (v/v), 25% (v/v) and 50% (v/v) ethanol-PBS, 75% (v/v) and 90% (v/v) ethanol-H2O (2×) followed by 50% ethanol-hexamethyldisilazane (HMDS) and 100% HMDS (Sigma-Aldrich, cat. no 379212). Coverslips were removed from the 100% HMDS, air-dried overnight at room temperature and mounted onto 12 mm specimen stubs (Agar Scientific). Following gold-coating to 1 nm using a Q150R sputter coater (Quorum Technologies), samples were examined with a Phenom PRO table-top scanning electron microscope (Phenom-World).

### Transmission Electron Microscopy (TEM)

Organoids were placed in BME on 3 mm diameter and 200 μm depth standard flat carriers for high pressure freezing and immediately cryoimmobilized using a Leica EM high-pressure freezer (equivalent to the HPM10), and stored in liquid nitrogen until further use. They were freeze-substituted in anhydrous acetone containing 2% osmium tetroxide 0.1% uranyl acetate at −90° C. for 72 hours and warmed to room temperature, 5° C. per hour (EM AFS-2, Leica, Vienna, Austria). The samples were kept 2 h at 4° C. and 2 h more at room temperature. After acetone rinses (4×15 min), Epon resin infiltration was performed during 2 days (acetone:resin 3:1-3 h; 2:2-3 h; 3:1-overnight; pure resin—6 h+overnight+6 h+overnight+3 h). Resin was polymerized at 60° C. during 96 hours. Leica Ultracut UC6 ultramicrotome was used to cut sections which were mounted on Formvar-coated copper grids and stained with 2% uranyl acetate. Sections were observed in a Tecnai T12 Spirit equipped with an Eagle 4 k×4 k camera (FEI Company, The Netherlands) and large EM overviews were collected using the principles and software described by Ravelli et al (67).

### REFERENCES

1. Squier C, Kremer M. Biology of oral mucosa and esophagus. J Natl Cancer Inst Monogr. 2001; 29:7-15.
2. Fitzmaurice C, Allen C, Barber R M, Barregard L, Bhutta Z A, Brenner H, et al. Global, Regional, and National Cancer Incidence, Mortality, Years of Life Lost, Years Lived With Disability, and Disability-Adjusted Life-years for 32 Cancer Groups, 1990 to 2015: A Systematic Analysis for the Global Burden of Disease Study. JAMA Oncol. United States; 2017; 3:524-48.
3. Leemans C R, Snijders P J F, Brakenhoff R H. The molecular landscape of head and neck cancer. Nat. Rev. Cancer. 2018.
4. Argiris A, Karamouzis M V, Raben D, Ferris R L. Head and neck cancer. Lancet (London, England). 2008; 371:1695-709.
5. Barretina J, Caponigro G, Stransky N, Venkatesan K, Margolin A A, Kim S, et al. The Cancer Cell Line Encyclopedia enables predictive modelling of anticancer drug sensitivity. Nature. 2012;
6. Smits J P H, Niehues H, Rikken G, Van Vlijmen-Willems I M J J, Van De Zande G W H J F, Zeeuwen P L J M, et al. Immortalized N/TERT keratinocytes as an alternative cell source in 3D human epidermal models. Sci Rep. 2017; 7.
7. Méry B, Rancoule C, Guy J B, Espenel S, Wozny A S, Battiston-Montagne P, et al. Preclinical models in HNSCC: A comprehensive review. Oral Oncol. 2017.
8. Laban S, Steinmeister L, Gleißner L, Grob T J, Grénman R, Petersen C, et al. Sorafenib sensitizes head and neck squamous cell carcinoma cells to ionizing radiation. Radiother Oncol. 2013;
9. Maushagen R, Reers S, Pfannerstill A C, Hahlbrock A, Stauber R, Rahmanzadeh R, et al. Effects of paclitaxel on permanent head and neck squamous cell carcinoma cell lines and identification of anti-apoptotic caspase 9b. J Cancer Res Clin Oncol. 2016;
10. Voskoglou-Nomikos T, Pater J L, Seymour L. Clinical predictive value of the in vitro cell line, human xenograft, and mouse allograft preclinical cancer models. Clin Cancer Res. 2003.
11. Johnson J I, Decker S, Zaharevitz D, Rubinstein L V., Venditti J M, Schepartz S, et al. Relationships between drug activity in NCl preclinical in vitro and in vivo models and early clinical trials. Br J Cancer. 2001;
12. Kross K W, Heimdal J H, Olsnes C, Olofsson J, Aarstad H J. Co-culture of head and neck squamous cell carcinoma spheroids with autologous monocytes predicts prognosis. Scand J Immunol. 2008;
13. Kohno N, Ohnuma T, Truog P. Effects of hyaluronidase on doxorubicin penetration into squamous carcinoma multicellular tumor spheroids and its cell lethality. J Cancer Res Clin Oncol. 1994;
14. Braakhuis B J, Sneeuwloper G, Snow G B. The potential of the nude mouse xenograft model for the study of head and neck cancer. Arch Otorhinolaryngol. 1984;
15. Smith L P, Thomas G R. Animal models for the study of squamous cell carcinoma of the upper aerodigestive tract: A historical perspective with review of their utility and limitations. Part A. Chemically-induced de novo cancer, syngeneic animal models of HNSCC, animal models of t. Int. J. Cancer. 2006.
16. Clarkson E, Mashkoor F, Abdulateef S. Oral Viral Infections: Diagnosis and Management. Dent. Clin. North Am. 2017.
17. Sayers C L, Elliott G. Herpes Simplex Virus 1 Enters Human Keratinocytes by a Nectin-1-Dependent, Rapid Plasma Membrane Fusion Pathway That 786 Functions at Low Temperature. J Virol. 2016;
18. D'Souza G, Kreimer A R, Viscidi R, Pawlita M, Fakhry C, Koch W M, et al. Case-Control Study of Human Papillomavirus and Oropharyngeal Cancer. N Engl J Med. 2007; 356:1944-56.
19. Griffin L M, Cicchini L, Xu T, Pyeon D. Human Keratinocyte Cultures in the Investigation of Early Steps of Human Papillomavirus Infection. Methods Mol Biol. 2014; 219-238.
20. Drost J, Clevers H. Organoids in cancer research. Nat Rev Cancer. England; 2018; 18:407-18.
21. Vlachogiannis G, Hedayat S, Vatsiou A, Jamin Y, Fernández-mateos J, Khan K, et al. Patient-derived organoids model treatment response of metastatic gastrointestinal cancers. Science (80-). 2018; 926:920-6.
22. Etienne L, Joshi P, Dingle L, Huang E, Grzesik P, Desai P J. Visualization of herpes simplex virus type 1 virions using fluorescent colors. J Virol Methods. 2017;
23. Gillison M L, Lowy D R. A causal role for human papillomavirus in head and neck cancer. Lancet. 2004;
24. Pyeon D, Lambert P F, Ahlquist P. Production of infectious human papillomavirus independently of viral replication and epithelial cell differentiation. Proc Natl Acad Sci. 2005;
25. Van De Wetering M, Francies H E, Francis J M, Bounova G, Iorio F, Pronk A, et al.
Prospective derivation of a living organoid biobank of colorectal cancer patients. Cell. Elsevier Inc.; 2015; 161:933-45.
26. Fujii M, Shimokawa M, Date S, Takano A, Matano M, Nanki K, et al. A Colorectal Tumor Organoid Library Demonstrates Progressive Loss of Niche Factor Requirements during Tumorigenesis. Cell Stem Cell. 2016; 18:827-38.
27. Vassilev L T, Vu B T, Graves B, Carvajal D, Podlaski F, Filipovic Z, et al. In Vivo Activation of the p53 Pathway by Small-Molecule Antagonists of MDM2. Science (80-). 2004;
28. The Cancer Genome Atlas Network. Comprehensive genomic characterization of head and neck squamous cell carcinomas. Nature. 2015; 517:576-82.
29. Freed-Pastor W A, Prives C. Mutant p53: One name, many proteins. Genes Dev. 2012
30. Love M I, Huber W, Anders S. Moderated estimation of fold change and dispersion for RNA-seq data with DESeq2. Genome Biol. 2014;
31. Schrader C H, Kolb M, Zaoui K, Flechtenmacher C, Grabe N, Weber K J, et al. Kallikrein related peptidase 6 regulates epithelial-to-mesenchymal transition and serves as prognostic biomarker for head and neck squamous cell carcinoma patients. Mol Cancer. 2015;
32. Zolk O, Schnepf R, Muschler M, Fromm M F, Wendler O, Traxdorf M, et al. Transporter Gene Expression in Human Head and Neck Squamous Cell Carcinoma and Associated Epigenetic Regulatory Mechanisms. Am J Pathol. 2013;
33. Castillo-González A C, Nieto-Cerón S, Pelegrín-Hernández J P, Montenegro M F, Noguera J A, López-Moreno M F, et al. Dysregulated cholinergic network as a novel biomarker of poor prognostic in patients with head and neck squamous cell carcinoma. BMC Cancer. 2015;
34. Bhosale P G, Cristea S, Ambatipudi S, Desai R S, Kumar R, Patil A, et al. Chromosomal Alterations and Gene Expression Changes Associated with the Progression of Leukoplakia to Advanced Gingivobuccal Cancer. Transl Oncol. 2017;
35. Yan L, Zhan C, Wu J, Wang S. Expression profile analysis of head and neck squamous cell carcinomas using data from The Cancer Genome Atlas. 833 Mol Med Rep. 2016;
36. Gonzalez H E, Gujrati M, Frederick M, Henderson Y, Arumugam J, Spring P W, et al. Identification of 9 genes differentially expressed in head and neck squamous cell carcinoma. Arch Otolaryngol Head Neck Surg. United States; 2003; 129:754-9.
37. Shimizu S, Tsukada J, Sugimoto T, Kikkawa N, Sasaki K, Chazono H, et al. Identification of a novel therapeutic target for head and neck squamous cell carcinomas: A role for the neurotensin-neurotensin receptor 1 oncogenic signaling pathway. Int J Cancer. 2008;
38. van Jaarsveld R H, Kops G J P L. Difference Makers: Chromosomal Instability versus Aneuploidy in Cancer. Trends in cancer. United States; 2016; 2:561-71.
39. Lengauer C, Kinzler K W, Vogelstein B. Genetic instability in colorectal cancers. Nature. 1997;
40. Koo B K, Stange D E, Sato T, Karthaus W, Farin H F, Huch M, et al. Controlled gene expression in primary Lgr5 organoid cultures. Nat Methods. 2012; 9:81-3.
41. Cavalieri S, Perrone F, Miceli R, Ascierto P A, Locati L D, Bergamini C, et al. Efficacy and safety of single-agent pan-human epidermal growth factor receptor (HER) inhibitor dacomitinib in locally advanced unresectable or metastatic skin squamous cell cancer. Eur J Cancer. Elsevier Ltd; 2018; 97:7-15.
42. Soulières D, Faivre S, Mesía R, Remenár É, Li S H, Karpenko A, et al. Buparlisib and paclitaxel in patients with platinum-pretreated recurrent or metastatic squamous cell carcinoma of the head and neck (BERIL-1): a randomised, double-blind, placebo controlled phase 2 trial. Lancet Oncol. 2017; 18:323-35.
43. Machiels J P H, Haddad R I, Fayette J, Licitra L F, Tahara M, Vermorken J B, et al. Afatinib versus methotrexate as second-line treatment in patients with recurrent or metastatic squamous-cell carcinoma of the head and neck progressing on or after platinum based therapy (LUX-Head & Neck 1): An open-label, randomised phase 3 trial. Lancet Oncol. 2015; 16:583-94.
44. Machiels J P, Bossi P, Menis J, Lia M, Fortpied C, Liu Y, et al. Activity and safety of afatinib in a window preoperative EORTC study in patients with squamous cell carcinoma of the head and neck (SCCHN). Ann Oncol. 2018; 29:985-91.
45. Bossi P, Resteghini C, Paielli N, Licitra L, Pilotti S, Perrone F. Prognostic and predictive value of EGFR in head and neck squamous cell carcinoma. Oncotarget. 2016; 7:74362-79.
46. Andersson A, Fagerberg J, Lewensohn R, Ehrsson H. Pharmacokinetics of cisplatin and its monohydrated complex in humans. J Pharm Sci. 1996; 85:824-7.
47. Perez R P, O'Dwyer P J, Handel L M, Ozols R F, Hamilton T C. Comparative cytotoxicity of CI-973, cisplatin, carboplatin and tetraplatin in human ovarian carcinoma cell lines. Int J Cancer. 1991; 48:265-9.
48. Atsushi H, Shuji S, Kosuke A, Takafumi K. A comparison of in vitro platinum-DNA adduct formation between carboplatin and cisplatin. Int J Biochem. 1994; 26:1009-16. 49. Reed E. Platinum-DNA adduct, nucleotide excision repair and platinum based anti-cancer chemotherapy. Cancer Treat. Rev. 1998. page 331-44.
50. Guan J, Li Q, Zhang Y, Xiao N, Chen M, Zhang Y, et al. A meta-analysis comparing cisplatin-based to carboplatin-based chemotherapy in moderate to advanced squamous cell carcinoma of head and neck (SCCHN). Oncotarget. 2016; 7:7110-9.
51. Mayer I A, Abramson V G, Formisano L, Balko J M, Estrada M V, Sanders M E, et al. A Phase Ib Study of Alpelisib (BYL719), a PI3Kα-specific Inhibitor, with Letrozole in ER+/HER2− Negative Metastatic Breast Cancer. Clin Cancer 880 Res. 2017; 23:26-34.
52. Zhu X, Ding X. Study on a 3D Hydrogel-Based Culture Model for Characterizing Growth of Fibroblasts under Viral Infection and Drug Treatment. SLAS Discov. 2017; 22:626-34.
53. Valyi-Nagy T, Fredericks B, Ravindra A, Hopkins J, Shukla D, Valyi-Nagy K. Herpes simplex virus type 1 infection promotes the growth of a subpopulation of tumor cells in 3D uveal melanoma cultures. J Virol. United States; 2018;
54. Cai Y, Dodhia S, Su G H. Dysregulations in the PI3K pathway and targeted therapies for head and neck squamous cell carcinoma. Oncotarget. 2017; 8:22203-17.
55. Munster P, Elkabets M, Gilbert J, Razak A R A, Ahn M J, Yen C J, et al. Abstract A46: Inhibition of PIK3C A with BYL719 can overcome resistance to cetuximab in squamous cell carcinoma of the head and neck (SCCHN). Mol Cancer Ther. 2015; 14:A46-A46.
56. Ho A, Chau N, IBrana Garcia I, Ferte C, Even C, Burrows F, et al. Preliminary results from a phase 2 trial of tipifarnib in HRAS-mutant head and neck squamous cell carcinomas. Int J Radiat Oncol Biol Phys. 2018;
57. Fritsch C, Huang A, Chatenay-Rivauday C, Schnell C, Reddy A, Liu M, et al. Characterization of the Novel and Specific PI3K Inhibitor NVP-BYL719 and Development of the Patient Stratification Strategy for Clinical Trials. Mol Cancer Ther. 2014;
58. Juric D, Rodon J, Tabernero J, Janku F, Burris H A, Schellens J H M, et al. Phosphatidylinositol 3-Kinase α-Selective Inhibition With Alpelisib (BYL719) in PIK3C A-Altered Solid Tumors: Results From the First-in-Human Study. J Clin Oncol. 2018;
59. Economopoulou P, Perisanidis C, Giotakis El, Psyrri A. The emerging role of immunotherapy in head and neck squamous cell carcinoma (HNSCC): anti-tumor immunity and clinical applications. Ann Transl Med. 2016; 4:173-173.
60. Dijkstra K K, Cattaneo C M, Weeber F, Chalabi M, van de Haar J, Fanchi L F, et al. Generation of Tumor-Reactive T Cells by Co-culture of Peripheral Blood Lymphocytes and Tumor Organoids. Cell. Elsevier; 2018; 174:1586-1598.e12.
61. Hashimshony T, Wagner F, Sher N, Yanai I. CEL-Seq: single-cell RNA-Seq by multiplexed linear amplification. Cell Rep. United States; 2012; 2:666-73.
62. Simmini S, Bialecka M, Huch M, Kester L, van de Wetering M, Sato T, et al. Transformation of intestinal stem cells into gastric stem cells on loss of transcription factor Cdx2. Nat Commun. England; 2014; 5:5728.
63. Li H, Durbin R. Fast and accurate long-read alignment with Burrows-Wheeler transform. Bioinformatics. England; 2010; 26:589-95.
64. Hoogstraat M, Hinrichs J W J, Besselink N J M, Radersma-Van Loon J H, De Voijs C M A, Peeters T, et al. Simultaneous detection of clinically relevant mutations and amplifications for routine cancer pathology. J Mol Diagnostics. 2015;
65. Shaner N C, Lambert G G, Chammas A, Ni Y, Cranfill P J, Baird M A, et al. A bright monomeric green fluorescent protein derived from Branchiostoma lanceolatum. Nat Methods. 2013; 10:407-9.
66. Huch M, Dorrell C, Boj S F, van Es J H, Li V S W, van de Wetering M, et al. In vitro expansion of single Lgr5+ liver stem cells induced by Wnt-driven regeneration. Nature. 2013; 494:247-50.
67. Faas F G A, Avramut M C, van den Berg B M, Mommaas A M, Koster A J, Ravelli R B G. Virtual nanoscopy: generation of ultra-large high resolution electron microscopy maps. J Cell Biol. United States; 2012; 198:457-69.

1. A method for culturing an epithelial stem cell or a population of epithelial stem cells derived from head or neck tissue, wherein the method comprises culturing one or more epithelial stem cells in contact with an extracellular matrix in the presence of an expansion medium, the expansion medium comprising a basal medium for animal or human cells and further comprising: one or more mitogenic growth factor, a TGF-beta inhibitor, an activator of the prostaglandin signalling pathway, one or more Wnt agonist, a cAMP pathway activator, a BMP inhibitor and nicotinamide.

2. The method of claim 1, wherein in the expansion medium the one or more Wnt agonist comprises an R-spondin family protein and/or a GSK-3 inhibitor, optionally wherein the R-spondin family protein is selected from R-spondin-1, R-spondin-2, R-spondin-3, R-spondin-4 or a fragment, analog or variant thereof.

3. The method of any one of the preceding claims, wherein the GSK-3 inhibitor is CHIR-99021, optionally at a final concentration of about 3 μM or about 0.3 μM.

4. The method of any one of the preceding claims, wherein in the expansion medium the one or more mitogenic growth factor binds to a receptor tyrosine kinase such as EGFR, an FGFR or HGFR, optionally wherein the one or more mitogenic growth factors are selected from EGF, FGF and HGF, optionally wherein the FGF comprises FGF2 and FGF10.

5. The method of any one of the preceding claims, wherein the one or more mitogenic growth factor comprises EGF, FGF10 and FGF2, optionally wherein:
the EGF is at a final concentration of about 50 ng/ml,
the FGF10 is at a final concentration of about 10 ng/ml, and/or
the FGF2 is at a final concentration of about 5 ng/ml.

6. The method of any one of the preceding claims, wherein in the expansion medium the TGF-beta inhibitor is an inhibitor of the ALK4, ALK5 or ALK7 signalling pathway, optionally wherein the TGF-beta inhibitor is a small molecule inhibitor, optionally A83-01, optionally wherein the final concentration of A83-01 is about 500 nM.

7. The method any one of the preceding claims, wherein in the expansion medium the activator of the prostaglandin signalling pathway is selected from the list comprising: a phospholipid, Arachidonic acid (AA), prostaglandin E2 (PGE2), prostaglandin G2 (PGG2), prostaglandin F2 (PGF2), prostaglandin H2 (PGH2), and prostaglandin D2 (PGD2).

8. The method of any one of the preceding claims, wherein the activator of the prostaglandin signalling pathway is PGE2, optionally wherein the final concentration PGE2 is about 1 μM.

9. The method of any one of the preceding claims, wherein in the expansion medium the cAMP pathway activator is an adenylyl cyclase activator, optionally selected from the list comprising: forskolin, a forskolin analog and cholera toxin.

10. The method of any one of the preceding claims, wherein the cAMP pathway activator is forskolin, optionally wherein the final concentration is about 1 μM.

11. The method of any one of the preceding claims, wherein in the expansion medium the BMP inhibitor is noggin, optionally wherein the final is about 100 ng/ml.

12. The method of any one of the preceding claims, wherein the final concentration of nicotinamide is between 1 μM and 500 mM, between 1 μM and 250 mM, between 100 μM and 250 mM, between 500 μM and 250 mM, between 1 mM and 250 mM, between 1 mM and 100 mM, or wherein the final concentration is about 10 mM.

13. The method of any one of the preceding claims, wherein the expansion medium further comprises B27 and/or N-acetyl-L-cysteine.

14. The method of any one of the preceding claims, wherein the mitogenic growth factor is EGF, FGF10 and FGF2, wherein the TGF-beta inhibitor is A83-01, wherein the Wnt agonist comprises R-spondin and CHIR-99021, wherein the cAMP pathway activator is forskolin, wherein the activator of the prostaglandin signalling pathway is PGE2, wherein the BMP inhibitor is noggin.

15. The method of claim 14, wherein the final concentration of nicotinamide is about 10 mM, the final concentration of EGF is about 50 ng/ml, the final concentration of FGF10 is about 10 ng/ml, the final concentration of FGF2 is about 5 ng/ml, the final concentration of A83-01 is about 500 nM, the final concentration of R-spondin is about 200 ng/ml, the final concentration of CHIR-99021 is about 3 μM or about 0.3 μM, the final concentration of forskolin is about 1 μM, the final concentration of PGE2 is about 1 μM, and the final concentration of noggin is about 100 ng/ml.

16. The method of any one of preceding claims, wherein the epithelial stem cell is part of an organoid or isolated from an organoid, or wherein the population of epithelial stem cells is an organoid, part of an organoid or isolated from an organoid.

17. The method of any one of the preceding claims, wherein the epithelial stem cell is a tumour cell or the population of epithelial stem cells are tumour cells.

18. The method of any one of preceding claims, wherein the method further comprises a step of culturing the expanded epithelial stem cells in a differentiation medium.

19. A method for culturing a mouse epithelial stem cell or a population of mouse epithelial stem cells, wherein the method comprises culturing one or more epithelial stem cells in contact with an extracellular matrix in the presence of an expansion medium, the expansion medium comprising a basal medium and further comprising: one or more mitogenic growth factors, one or more Wnt agonists and nicotinamide; wherein the epithelial stem cells are derived from head or neck tissue.

20. The method of any one of the preceding claims, wherein the method further comprises culturing for a sufficient number of days to obtain an organoid.

21. An expansion medium as defined in any one of claims 1-20.

22. An organoid obtainable or obtained by a method of any one of claims 1-20, optionally wherein the organoid is a tumour organoid.

23. A composition comprising an expansion medium according to claim 21 and an organoid or tumour organoid according to claim 22.

24. Use of an organoid of claim 22 as a model for infection, optionally wherein the infection is viral infection.

25. A method of modelling viral infection comprising the step of culturing an epithelial stem cell or a population of epithelial stem cells derived from head or neck tissue in accordance with any one of claims 1-20, and further comprising infecting the resultant organoid or population of cells with a virus, optionally wherein the virus is a Herpes Simplex Virus, such as HSV1, or a Human Papilloma Virus, such as HPV16.

26. An organoid as defined in claim 22 for use in diagnostics or medicine, optionally in personalised medicine or diagnostics, or regenerative medicine.

27. Use of an organoid of claim 22 in drug screening, target validation, target discovery, toxicology, toxicology screens or an ex vivo cell/organ model.

28. An organoid of claim 22 for use in a method to guide personalised therapy.

29. Use of an organoid of claim 22 in an ex vivo method to predict a clinical outcome.

30. A method for testing the effect of a candidate compound, wherein the method comprises:
the method of any one of claims 1-20;
exposing the resultant population of cells or the resultant organoid to one or a library of candidate compounds;
evaluating said expanded organoids for any effects,
identifying the candidate molecule that causes said effects as a potential drug; and optionally providing said candidate molecule, e.g. as a drug.

31. A method for selecting a treatment regime for a patient, wherein the method comprises the steps of:
optionally obtaining a biopsy from the head or neck tissue of the patient;
culturing the biopsy, a tissue fragment of the biopsy, an epithelial stem cell of the biopsy or a population of epithelial stem cells of the biopsy according to the method of any one of claims 1-20 to obtain an organoid or a tumour organoid;
exposing the resultant organoid to a treatment regime, including radiation and/or one or more candidate compounds;
evaluating said organoid for any effects;
identifying the treatment of regime that causes said effects; and
optionally providing said treatment to the patient.
